(19) United States
(12) Patent Application Publication
(10) Pub. No.: US 2011/0044988 A1
(43) Pub. Date: Feb. 24, 2011

(54) METHODS OF TREATMENT USING ANTI-MIF ANTIBODIES

(75) Inventors: Jürgen Bernhagen, Aachen (DE); Joshua Robert Schultz, Ballston Lake, NY (US); Benedikt Vollrath, San Diego, CA (US); Alma Zernecke, Aachen (DE); Christian Weber, Aachen (DE)

Correspondence Address: WILSON, SONSINI, GOODRICH & ROSATI, 650 PAGE MILL ROAD, PALO ALTO, CA, 94304-1050, US

(73) Assignee: CAROLUS THERPEUTICS, INC., San Diego, CA (US)

(21) Appl. No.: 12/918,968

(22) PCT Filed: Mar. 20, 2009
(86) PCT No.: PCT/US09/37883

§ 371 Date: Nov. 8, 2010

Related U.S. Application Data
(60) Provisional application No. 61/038,381, filed on Mar. 20, 2008.
(60) Provisional application No. 61/039,371, filed on Mar. 25, 2008.
(60) Provisional application No. 61/045,807, filed on Apr. 17, 2008.
(60) Provisional application No. 61/121,095, filed on Dec. 9, 2008.

(51) Int. Cl. A61K 39/395; C07K 16/24; A61P 9/10; A61P 11/00; A61P 37/06; A61P 3/10; A61P 35/00; A61P 25/00; A61P 25/16

(52) U.S. Cl. 424/139.1; 424/158.1; 424/141.1; 530/389.2; 530/387.9

(57) ABSTRACT

Disclosed herein, in certain embodiments, is a method for treating an inflammatory disorder. In some embodiments, the method comprises administering an active agent that inhibits (i) MIF binding to CXCR2 and CXCR4 and/or (ii) MIF-activation of CXCR2 and CXCR4; (iii) the ability of MIF to form a homomultimer; or a combination thereof.

### CROSS-REFERENCE

This application claims the benefit of U.S. Provisional Application No. 61/038,381, filed Mar. 20, 2008; U.S. Provisional Application No. 61/039,371, filed Mar. 25, 2008; U.S. Provisional Application No. 61/045,807, filed Apr. 17, 2008; and U.S. Provisional Application No. 61/121,095, filed Dec. 9, 2008; which applications are incorporated herein by reference in their entirety.

### BACKGROUND OF THE INVENTION

Certain inflammatory conditions are characterized, in part, by the migration lymphocytes into the effected tissue. The migration of lymphocytes induces tissue damage and exacerbates inflammatory conditions. Many leukocytes follow a MIF gradient to the effected tissue. In general, MIF interacts with CXCR2 and CXCR4 receptors on leukocytes to trigger and maintain leukocyte migration.

### SUMMARY OF THE INVENTION

Disclosed herein, in certain embodiments, is a method of treating a MIF-mediated disorder comprising administering to an individual in need thereof a therapeutically-effective amount of an antibody that inhibits (i) MIF binding to CXCR2 and/or CXCR4 (ii) MIF-activation of CXCR2 and/or CXCR4; (iii) the ability of MIF to form a homomultimer; (iv) MIF binding to CD74; or a combination thereof. In some embodiments, the antibody specifically binds to all or a portion of a pseudo-ELR motif of MIF. In some embodiments, the antibody specifically binds to all or a portion of an N-Loop motif of MIF. In some embodiments, the antibody specifically binds to all or a portion of the pseudo-ELR and N-Loop motifs of MIF. In some embodiments, the antibody is selected from an anti-CXCR2 antibody; an anti-CXCR4 antibody; an anti-MIF antibody; an antibody that specifically binds to all or a portion of the pseudo-ELR motif of MIF; an antibody that specifically binds to all or a portion of the N-loop motif of MIF; an antibody that specifically binds to all or a portion of the pseudo-ELR and N-Loop motifs; an antibody that inhibits the binding of MIF and CXCR2; an antibody that inhibits the binding of MIF and CXCR4; and antibody that inhibits the binding of MIF and JAB-1; an antibody that inhibits the binding of MIF and CD74; an antibody that specifically binds to all or a portion of a peptide sequence as follows: DQLMAFGGSSEPCALCSL and the corresponding feature/domain of at least one of a MT monomer or MIF trimer; an antibody that specifically binds to all or a portion of a peptide sequence as follows: PRASVPDGFLSELTQQLAQATGKPPQYIAVHVVPDQLMAFGGSSEPCALCSL and the corresponding feature/domain of at least one of a MIF monomer or MIF trimer; an antibody that specifically binds to all or a portion of a peptide sequence as follows: FGGSSEPCALCSLHSI and the corresponding feature/domain of at least one of a MIF monomer or MIF trimer; or combinations thereof. In some embodiments, the antibody is selected from anti-CXCR4 antibodies: 701, 708, 716, 717, 718, 12G5 and 4G10; anti-MIF antibodies: IID.9, IIID.9, XIF7, I31, IV2.2, XI17, XIV14.3, XII15.6 and XIV15.4; or combinations thereof. In some embodiments, the conversion of a macrophage into a foam cell is inhibited following administration of an antibody disclosed herein. In some embodiments, apoptosis of a cardiac myocyte is inhibited following administration of an antibody disclosed herein. In some embodiments, apoptosis of an infiltrating macrophage is inhibited following administration of an antibody disclosed herein. In some embodiments, the formation of an abdominal aortic aneurysm is inhibited following administration of an antibody disclosed herein. In some embodiments, the diameter of an abdominal aortic aneurysm is decreased following administration of an antibody disclosed herein. In some embodiments, a structural protein in an aneurysm is regenerated following administration of an antibody disclosed herein. In some embodiments, the method further comprises co-administering a second active agent. In some embodiments, the method further comprises co-administering niacin, a fibrate, a statin, a Apo-A1 mimetic peptide (e.g., DF-4, Novartis), an apoA-I transcriptional up-regulator, an ACAT inhibitor, a CETP modulator, Glycoprotein (GP) IIb/IIIa receptor antagonists, P2Y12 receptor antagonists, Lp-PLA2-inhibitors, an anti-TNF agent, an IL-1 receptor antagonist, an IL-2 receptor antagonist, a cytotoxic agent, an immunomodulatory agent, an antibiotic, a T-cell co-stimulatory blocker, a disorder-modifying anti-rheumatic agent, a B cell depleting agent, an immunosuppressive agent, an anti-lymphocyte antibody, an alkylating agent, an anti-metabolite, a plant alkaloid, a terpenoids, a topoisomerase inhibitor, an anti-tumor antibiotic, a monoclonal antibody, a hormonal therapy, or combinations thereof. In some embodiments, the MIF-mediated disorder is Atherosclerosis; Abdominal aortic aneurysm Acute disseminated encephalomyelitis; Moyamoya disease; Takayasu disease; Acute coronary syndrome; Cardiac-allograft vasculopathy; Pulmonary inflammation; Acute respiratory distress syndrome; Pulmonary fibrosis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré Syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer, Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Septic shock; Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Neointimal hyperplasia, Myocardial infarction; Stroke; Organ transplant rejection; or combinations thereof. In some embodiments, the disorder is an abdominal aortic aneurysm. In some embodiments, the disorder is atherosclerosis.

Disclosed herein, in certain embodiments, is a pharmaceutical composition for treatment of a MIF-mediated disorder, comprising an antibody that inhibits (i) MIF binding to CXCR2 and CXCR4; MIF-activation of CXCR2 and CXCR4; (iii) the ability of MIF to form a homomultimer; or a combination thereof. In some embodiments, the antibody specifically binds to all or a portion of a pseudo-ELR motif of MIF. In some embodiments, the antibody specifically binds to all or a portion of a N-Loop motif of MIF. In some embodiments, the antibody specifically binds to all or a portion of the pseudo-ELR and N-Loop motifs of MIF. In some embodiments, the antibody is selected from an anti-CXCR2 antibody; an anti-CXCR4 antibody, an anti-MIF antibody; an antibody that specifically binds to all or a portion of the pseudo-ELR motif of MIF; an antibody that specifically binds to all or a portion of the N-loop motif of MIF; an antibody that specifically binds to all or a portion of the pseudo-ELR and N-Loop motifs; an antibody that inhibits the binding of MIF and CXCR2; an antibody that inhibits the binding of MIF and CXCR4; and antibody that inhibits the binding of MIF and JAB-1; an antibody that inhibits the binding of MIF and CD74; an antibody that specifically binds to all or a portion of a peptide sequence as follows: DQLMAFGGSSEPCALCSL and the corresponding feature/domain of at least one of a MIF monomer or MIF trimer; an antibody that specifically binds to all or a portion of a peptide sequence as follows: PRASVPDGFLSELTQQLAQATGKPPQYIAVHVVPDQLMAFGGSSEPCALCSL and the corresponding feature/domain of at least one of a MIF monomer or MIF trimer; an antibody that specifically binds to all or a portion of a peptide sequence as follows: FGGSSEPCALCSLHSI and the corresponding feature/domain of at least one of a MIF monomer or MIF trimer; or combinations thereof. In some embodiments, the antibody is selected from anti-CXCR4 antibodies 701, 708, 716, 717, 718, 12G5 and 4G10; anti-MIF antibodies IID.9, IIID.9, XIF7, I31, IV2.2, XI17, XIV14.3, XII15.6 and XIV15.4; or combinations thereof. In some embodiments, the composition further comprises a second active agent. In some embodiments, the composition further comprises niacin, a fibrate, a statin, a Apo-A1 mimetic peptide (e.g., DF-4, Novartis), an apoA-I transcriptional up-regulator, an ACAT inhibitor, a CETP modulator, Glycoprotein (GP) IIb/IIIa receptor antagonists, P2Y12 receptor antagonists, Lp-PLA2-inhibitors, an anti-TNF agent, an IL-1 receptor antagonist, an IL-2 receptor antagonist, a cytotoxic agent, an immunomodulatory agent, an antibiotic, a T-cell co-stimulatory blocker, a disorder-modifying anti-rheumatic agent, a B cell depleting agent, an immunosuppressive agent, an anti-lymphocyte antibody, an alkylating agent, an anti-metabolite, a plant alkaloid, a terpenoids, a topoisomerase inhibitor, an anti-tumor antibiotic, a monoclonal antibody, a hormonal therapy, or combinations thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustration that MIF-triggered mononuclear cell arrest is mediated by CXCR2/CXCR4 and CD74. Human aortic endothelial cells (HAoECs), CHO cells stably expressing ICAM-1 (CHO/ICAM-1) and mouse microvascular endothelial cells (SVECs) were preincubated with or without MIF (together with antibody to MIF, antibodies to CXCL1 and CXCL8, or isotype control), CXCL8, CXCL10 or CXCL12 for 2 h as indicated. Mononuclear cells were pretreated with antibodies to CXCR1, CXCR2, β₂, CXCR4, CD74, or isotype controls for 30 min, or pertussis toxin (PTX) for 2 h as indicated. (a) HAoECs were perfused with primary human monocytes. (b) Immunofluorescence using antibody to MIF revealed surface presentation of MIF (green) on HAoECs and CHO/ICAM-1 cells after pretreatment for 2 h, but not 30 min (not shown); in contrast, MIF was absent in buffer-treated cells (control). Scale bar, 100 μm. (c,d) CHO/ICAM-1 cells were perfused with MonoMac6 cells. (e) HAoECs were perfused with T cells. (f,g) CHO/ICAM-1 cells were perfused with Jurkat T cells (0, and with Jurkat CXCR2 transfectants or vector controls (g). In c, d, f and g, background binding to vector-transfected CHO cells was subtracted. (h) Mouse SVECs were perfused with L1.2 transfectants stably expressing CXCR1, CXCR2 or CXCR3, and with controls expressing only endogenous CXCR4, in the presence of the CXCR4 antagonist AMD3465. Arrest is quantified as cells/mm² or as percentage of control cell adhesion. Data in a and c-g represent mean±s.d. of 3-8 independent experiments; data in h are results from one representative experiment of four experiments.

FIG. 2 is an illustration that MIF-triggered mononuclear cell chemotaxis is mediated by CXCR2/CXCR4 and CD74. Primary human monocytes (a-e), CD3* T cells (f) and neutrophils (g,h) were individualed to transmigration analysis in the presence or absence of MIF. CCL2 (a), CXCL8 (a,g,h) and CXCL12 (f) served as positive controls or were used to test desensitization by MIF (or by CXCL8, h). The chemotactic effects of MIF, CCL2 and CXCL8 on monocytes (a) or of MIF on neutrophils (g) followed bell-shaped dose-response curves. MIF-triggered chemotaxis of monocytes was abrogated by an antibody to MIF, boiling (b), or by MIF at indicated concentrations (in the top chamber; c). (d) MIF-triggered chemotaxis was mediated by G_{αi}/phosphoinositide-3-kinase signaling, as evidenced by treatment with pertussis toxin components A and B (PTX A+B), PTX component B alone or Ly294002. (e) MIF-mediated monocyte chemotaxis was blocked by antibodies to CD74 or CXCR1/CXCR2. (f) T-cell chemotaxis induced by MIF was blocked by antibodies to MIF and CXCR4. (g) Neutrophil chemotaxis induced by MIF. (h) MIF-induced versus CXCL8-induced neutrophil chemotaxis, effects of antibodies to CXCR2 or CXCR1, and desensitization of CXCL8 by MIF. Data in a and f-h are expressed as chemotactic index; data in c are expressed as percent of control; and data in b,d and e as percent of input. Data represent mean±s.d. of 4-10 independent experiments, except for panels a, c and g, boiled MIF in b, and the antibody-alone controls in b and e, which are means of 2 independent experiments.

FIG. 3 is an illustration that MIF triggers rapid integrin activation and calcium signaling. (a) Human aortic endothelial cells were stimulated with MIF or TNF-α for 2 h. CXCL1 and CXCL8 mRNAs were analyzed by real-time PCR and normalized to control. Supernatant-derived CXCL8 was assessed by ELISA (n=3 independent experiments performed in duplicate). (b) MonoMac6 cells were directly stimulated with MIF or CXCL8 for 1 min and perfused on CHO-ICAM-1 cells for 5 min (mean±s.d. of 8 independent experiments). (c) MonoMac6 cells were stimulated with MIF for the indicated times. LFA-1 activation (detected by the 327C antibody) was monitored by FACSAria, and expressed as the increase in mean fluorescence intensity (MFI). (d) As in c but for primary monocytes; data are expressed relative to maximal activation with Mg²⁺/EGTA. (e) MonoMac6 cells were pretreated with antibodies to α₄ integrin, CD74 or CXCR2, stimulated with MIF for 1 min, perfused on VCAM-1.Fc for 5 min. Adhesion is expressed as a percentage of controls. Arrest data in c-e represent mean±s.d. of 5 independent experiments. (f) Calcium transients in Fluo-4 AM-labeled neutrophils were stimulated with MIF, CXCL8 or CXCL7. Calcium-derived MFI was recorded by FACSAria for 0-240 s. For desensitization, stimuli were added 120 s before stimulation. Traces shown represent 4 independent experiments. (g) Dose-response curves of calcium-influx triggered by CXCL8, CXCL7 or MIF, at indicated concentrations, in L1.2-CXCR2 transfectants. Data are expressed as the difference between baseline and peak MFI (mean±s.d. of 4-8 independent experiments).

FIG. 4 is an illustration of MIF-interaction with CXCR2/CXCR4 and formation of CXCR2/CD74 complexes. HEK293-CXCR2 transfectants (a) or CXCR4-bearing Jurkat T-cells (c) were individualed to receptor binding assays, analyzing competition of [I¹²⁵]CXCL8 (a) or [I¹²⁵]CXCL12 (c) by MIF or cold cognate ligand (mean±s.d., n=6-10). (b) MIF- and CXCL8-induced CXCR2 internalization in HEK293-CXCR2 or RAW264.7-CXCR2 transfectants (inset shows representative histograms) as indicated; assessed by FACS analysis of surface CXCR2 expression (percentage of buffer (Con), mean±s.d., n=5). (d) MIF- and CXCL12-induced CXCR4 internalization in Jurkat T-cells as in b (mean±s.d., n=4-6). (e) Binding of fluorescein-MIF to HEK293-CXCR2 transfectants or vector controls analyzed by FACS. Inset shows binding of biotin-MIF to CXCR2 assessed by western blot using antibodies to CXCR2 after streptavidin (SAv) pull-down from HEK293-CXCR2 transfectants versus vector controls. (f) Colocalization of CXCR2 and CD74 (orange-yellow overlay) in RAW264.7-CXCR2 transfectants stained for CXCR2, CD74 and nuclei (Hoechst), analyzed by fluorescence microscopy (top) or confocal laser scanning microscopy (bottom). Scale bar, 10 μm. (g) Coimmunoprecipitation of CXCR2/CD74 complexes in CHAPSO-extracts of HEK293-CXCR2 transfectants expressing His-tagged CD74. Anti-His immunoprecipitation (IP) followed by anti-CXCR2 or anti-His-CD74 western blotting (WB; top) or anti-CXCR2 immunoprecipitation followed by anti-His-CD74 or anti-CXCR2 western blotting (bottom). Controls: lysates without immunoprecipitation or beads alone. (h) As in g for L1.2-CXCR2 transfectants. Anti-CXCR2 immunoprecipitation from L1.2-CXCR2 transfectants followed by anti-CD74 or anti-CXCR2 western blotting (top). Immunoprecipitation with isotype IgG or CXCR2-negative L1.2-cells (bottom) served as controls. Data represent 3 independent experiments (e-h).

FIG. 5 is an illustration that MN-induced atherogenic and microvascular inflammation through CXCR2 in vivo and effects of MIF blockade on plaque regression. (a) Monocyte adhesion to the lumen in vivo and lesional macrophage content in native aortic roots were determined in Mif^{+/+}Ldlr^{-/} and Mif^{-/}Ldlr^{-/} mice (n=4) fed a chow diet for 30 weeks. Representative images are shown. Arrows indicate monocytes adherent to the luminal surface. Scale bar, 100 μm. (b,c) Exposure to MIF induced CXCR2-dependent leukocyte recruitment in vivo. Following intrascrotal injection of MIF, the cremasteric microvasculature was visualized by intravital microscopy. Pretreatment with blocking CXCR2 antibody abrogated adhesion and emigration, as compared to IgG control (n=4). (d) Intraperitoneal injection of MIF or vehicle elicited neutrophil recruitment in wild-type mice (n=3) reconstituted with wild-type, but not Il8rb^{-/}, bone marrow. (e-h) Blocking MIF but not CXCL1 or CXCL12 resulted in regression and stabilization of advanced atherosclerotic plaques. Apoe^{-/} mice received a high-fat diet for 12 weeks and were subsequently treated with antibodies to MIF, CXCL1 or CXCL12, or with vehicle (control) for an additional 4 weeks of (n=6-10 mice). Plaques in the aortic root were stained using Oil-Red-O. Representative images are shown in e (scale bars, 500 μm). Data in f represent plaque area at baseline (12 weeks) and after 16 weeks. The relative content of MOMA-2⁺ macrophages is shown in g and the number of CD3⁺ T cells per section in h. Data represent mean±s.d.

FIG. 6 is an illustration of cellular mechanisms of MIF in the context of atherogenesis. MIF expression is induced in cells of the vascular wall and intimal macrophages by various proatherogenic stimuli, e.g., oxidized LDL (oxLDL) or angiotensin II (ATII). Subsequently, MIF upregulates endothelial cell adhesion molecules (e.g., vascular [VCAM-1] and intracellular [ICAM-1] adhesion molecules) and chemokines (e.g., CCL2) and triggers direct activation of the respective integrin receptors (e.g., LFA-1 and VLA-4) by binding and signaling through its heptahelical (chemokine) receptors CXCR2 and CXCR4. This entails the recruitment of mononuclear cells (monocytes and T cells) and the conversion of macrophages into foam cells, inhibiting apoptosis and regulating (e.g., impairing) the migration or proliferation of SMCs. By inducing MMPs and cathepsins, MIF promotes elastin and collagen degradation, ultimately leading to the progression into unstable plaques. ROS indicates reactive oxygen species; PDGF-BB, platelet-derived growth factor-BB.

FIG. 7 is an illustration of signaling via a functional MIF receptor complex. MIF is induced by glucocorticoids overriding their function by regulating cytokine production and, after its endocytosis, can interact with intracellular proteins, namely JAB-1, thereby downregulating MAPK signals and modulating cellular redox homeostasis. In some embodiments, extracellular MIF binds to the cell surface protein CD74 (invariant chain Ii). CD74 lacks a signal-transducing intracellular domain but interacts with the proteoglycan CD44 and mediates signaling via CD44 to induce activation of Src-family RTK and MAPK/extracellular signal-regulated kinase (ERK), to activate the PI3K/Akt pathway, or to initiate p53-dependent inhibition of apoptosis. MIF also binds and signals through G protein-coupled chemokine receptors (CXCR2 and CXCR4) alone. Complex formation of CXCR2 with CD74, enabling accessory binding, facilitates GPCR activation and formation of a GPCR-RTK-like signaling complex to trigger calcium influx and rapid integrin activation.

FIG. 8 is an illustration of the effects of MIF in myocardial pathology. In the context of ischemia-reperfusion, hypoxia, reactive oxygen species (ROS), and endotoxins (e.g., lipopolysaccharide [LPS]) in sepsis induce the secretion of MIF from cardiomyocytes through a protein kinase C (PKC)-dependent mechanism and result in extracellular signal-regulated kinase (ERK) activation, which contributes to cardiomyocyte apoptosis. Expressed by surviving cardiomyocytes or by endothelial progenitor cells (e.g., eEPCs) used for therapeutic injection, in some embodiments MIF promotes angiogenesis via its receptors CXCR2 and CXCR4, requiring MAPK and PI3K activation.

FIG. 9 is an illustration that interference with CXCR4 without concomitant interference with CXCR2 aggravates atherosclerosis. Apoe−/− mice receiving a high-fat diet were continuously treated with vehicle (control) or AMD3465 via osmotic minipumps for 12 weeks (n=6 each). Atherosclerotic plaques were quantified in the aortic root (FIG. 14a) and thoracoabdominal aorta (FIG. 14b) after oil red O staining. The relative number of neutrophils was determined by flow cytometric analysis or standard cytometry in peripheral blood at the indicated time points (FIG. 14C).

FIG. 10 illustrates the crystal structure of a MIF trimer. The pseudo-ELR domains form a ring in the trimer while the N-loop domains extend outward from the pseudo-ELR ring.

FIG. 11 illustrates the nucleotide sequence of MIF annotated to show the sequences that correspond to the N-Loop domain and the pseudo-ELR domain.

### DETAILED DESCRIPTION OF THE INVENTION

Disclosed herein, in certain embodiments, are methods of inhibiting MIF signaling through CXCR2 and CXCR4. In some embodiments, MIF signaling through CXCR2 and CXCR4 is inhibited by occupying the MIF binding domain of CXCR2 and CXCR4 with an antibody. In some embodiments MIF signaling through CXCR2 and CXCR4 is inhibited by occupying, masking, or otherwise disrupting domains on MIF. In some embodiments, MIF signaling through CXCR2 and CXCR4 is inhibited by an antibody occupying, masking, or otherwise disrupting domains on MIF and thereby disrupting the binding of CXCR2 and/or CXCR4 to MIF. In some embodiments, MIF signaling through CXCR2 and CXCR4 is inhibited by an antibody occupying, masking, or otherwise disrupting domains on MIF and thereby disrupting MIF trimerization.

While the art teaches anti-MIF antibodies, the art lacks recognition that certain portions of MIF are more important than others with respect to leukocyte interactions. A problem solved herein is the identification and raising of antibodies that bind the selective portions of MIF that are important to leukocyte chemotaxis.

Further, the art teaches anti-CXCR2 and anti-CXCR4 antibodies. However, these receptors are also involved in interactions with other ligands (e.g., IL-8/CXCL8, GRObeta/CXCL2 and/or Stromal Cell-Derived Factor-1a (SDF-1a)/CXCL12). Detrimental side-effects often arise if these interactions are inhibited. A problem solved herein is the failure of the art to design anti-CXCR2 and anti-CXCR4 antibodies that selectively inhibit interactions with MIF.

### Certain Definitions

The terms “individual,” “subject,” or “patient” are used interchangeably. As used herein, they mean any mammal (i.e. species of any orders, families, and genus within the taxonomic classification animalia: chordata: vertebrate: mammalia). In some embodiments, the mammal is a human. In some embodiments, the mammal is a non-human. In some embodiments, the mammal is a member of the taxonomic orders: primates (e.g. lemurs, lorids, galagos, tarsiers, monkeys, apes, and humans); rodentia (e.g. mice, rats, squirrels, chipmunks, and gophers); lagomorpha (e.g. hares, rabbits, and pika); erinaceomorpha (e.g. hedgehogs and gymnures); soricomorpha (e.g. shrews, moles, and solenodons); chiroptera (e.g., bats); cetacea (e.g. whales, dolphins, and porpoises); carnivora (e.g. cats, lions, and other feliformia; dogs, bears, weasels, and seals); perissodactyla (e.g. horse, zebra, tapir, and rhinoceros); artiodactyla (e.g. pigs, camels, cattle, and deer); proboscidea (e.g. elephants); sirenia (e.g. manatees, dugong, and sea cows); cingulata (e.g. armadillos); pilosa (e.g. anteaters and sloths); didelphimorphia (e.g. american opossums); paucituberculata (e.g. shrew opossums); microbiotheria (e.g. Monito del Monte); notoryctemorphia (e.g. marsupial moles); dasyuromorphia (e.g. marsupial carnivores); peramelemorphia (e.g. bandicoots and bilbies); or diprotodontia (e.g. wombats, koalas, possums, gliders, kangaroos, wallaroos, and wallabies). In some embodiments, the animal is a reptile (i.e. species of any orders, families, and genus within the taxonomic classification animalia: chordata: vertebrata: reptilia). In some embodiments, the animal is a bird (i.e. animalia: chordata: vertebrata: ayes). None of the terms require or are limited to situation characterized by the supervision (e.g. constant or intermittent) of a health care worker (e.g. a doctor, a registered nurse, a nurse practitioner, a physician's assistant, an orderly, or a hospice worker).

The term “antigen” refers to a substance that is capable of inducing the production of an antibody. In some embodiments an antigen is a substance that specifically binds to an antibody variable region.

The terms “antibody” and “antibodies” refer to monoclonal antibodies, polyclonal antibodies, bi-specific antibodies, multispecific antibodies, grafted antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies and antigen-binding fragments of any of the above. In particular, antibodies include immunoglobulin molecules and immunologically active fragments of immunoglobulin molecules, i.e., molecules that contain an antigen binding site. Depending on the amino acid sequence of the constant domain of their heavy chains, immunoglobulins can be assigned to different classes. The heavy-chain constant domains (Fc) that correspond to the different classes of immunoglobulins are called α, δ, ε, γ, and μ, respectively. The subunit structures and three-dimensional configurations of different classes of immunoglobulins are well known. Immunoglobulin molecules are of any type (e.g., IgG, IgE, IgM, IgD, IgA and IgY), class (e.g., IgG₁, IgG₂, IgG₃, IgG₄, IgA₁ and IgA₂) or subclass. The terms “antibody” and “immunoglobulin” are used interchangeably in the broadest sense. In some embodiments an antibody is part of a larger molecule, formed by covalent or non-covalent association of the antibody with one or more other proteins or peptides.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3 and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669).

The terms “hypervariable region” and “CDR” when used herein, refer to the amino acid residues of an antibody which are responsible for antigen-binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2) and 89-97 (CDRL3), and in the heavy chain variable domain the CDRs typically correspond to approximately residues 31-35 (CDRH1), 50-65 (CDRH2) and 95-102 (CDRH3) according to Kabat et al., Sequences of Proteins of Immunological Interest, 5th Ed. Public Health Service, National Institutes of Health, Bethesda, Md. (1991)). It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2) and 91-96 (CDRL3), and in the heavy chain variable domain, the CDRs typically correspond to approximately residues 26-32 (CDRH1), 53-55 (CDRH2) and 96-101 (CDRH3) according to Chothia and Lesk, J. Mol. Biol., 196: 901-917 (1987)).

As used herein, “framework region” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Sequences of Proteins of Immunological Interest, 5th Ed. Public Health Service, National Institutes of Health, Bethesda, Md. (1991)). As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, J. Mol. Biol., 196: 901-917 (1987)).

The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three dimensional structure of the antibody variable domain can be derived from a crystal structure or protein modeling.

Constant domains (Fc) of antibodies are not involved directly in binding an antibody to an antigen but, rather, exhibit various effector functions, such as participation of the antibody in antibody-dependent cellular toxicity via interactions with, for example, Fc receptors (FcR). Fc domains can also increase bioavailability of an antibody in circulation following administration to a patient.

The “light chains” of antibodies (immunoglobulins) from any vertebrate species can be assigned to one of two clearly distinct types, called kappa or (“κ”) and lambda or (“λ”), based on the amino acid sequences of their constant domains.

The term “derivative” in the context of an antibody refers to an antibody that comprises an amino acid sequence which has been altered by the introduction of amino acid residue substitutions, deletions or additions. The term “derivative” also refers to an antibody which has been modified, i.e., by the covalent attachment of any type of molecule to the antibody. For example, in some embodiments an antibody is modified, e.g., by glycosylation, acetylation, pegylation, phosphorylation, amidation, derivatization by protecting/blocking groups, proteolytic cleavage, linkage to a cellular ligand or other protein, etc. In some embodiments, antibodies and derivatives thereof are produced by chemical modifications using suitable techniques, including, but not limited to specific chemical cleavage, acetylation, formylation, metabolic synthesis of tunicamycin, etc. In some embodiments, a derivative of an antibody possesses a similar or identical function as the antibody from which it was derived.

The terms “full length antibody,” “intact antibody” and “whole antibody” are used herein interchangeably, to refer to an antibody in its substantially intact form, and not antibody fragments as defined below. These terms particularly refer to an antibody with heavy chains contains Fc regions. In some embodiments an antibody variant provided herein is a full length antibody. In some embodiments the full length antibody is human, humanized, chimeric, and/or affinity matured.

An “affinity matured” antibody is one having one or more alteration in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by suitable procedures. See, for example, Marks et al., (1992) Biotechnology 10:779-783 that describes affinity maturation by variable heavy chain (VH) and variable light chain (VL) domain shuffling. Random mutagenesis of CDR and/or framework residues is described in: Barbas, et al. (1994) Proc. Nat. Acad. Sci, USA 91:3809-3813; Shier et al., (1995) Gene 169:147-155; Yelton et al., 1995, J. Immunol. 155:1994-2004; Jackson et al., 1995, J. Immunol. 154(7):3310-9; and Hawkins et al, (19920, J. Mol. Biol. 226:889-896, for example.

The terms “binding fragment,” “functional fragment,” “antibody fragment” or “antigen binding fragment” are used herein, for purposes of the specification and claims, to mean a portion or fragment of an intact antibody molecule, preferably wherein the fragment retains antigen-binding function. Examples of antibody fragments include Fab, Fab′, F(ab′)₂, Fd (V_{H} and C_{H1} domains), Fd′ and Fv (the V_{L} and V_{H} domains of a single arm of an antibody) fragments, diabodies, linear antibodies (Zapata et al. (1995) Protein Eng. 10: 1057), variable light chains (VL), variable heavy chains (VH), single-chain antibody molecules, single-chain binding polypeptides, scFv, scFv2 (a tandem linkage of two scFv molecules head to tail in a chain), bivalent scFv, tetravalent scFv, one-half antibodies, dAb fragments, variable NAR domains, and bispecific or multispecific antibodies formed from antibody fragments (e.g., a bi-specific Fab₂, and a tri-specific Fab₃, etc.).

“Fab” fragments are typically produced by papain digestion of antibodies resulting in the production of two identical antigen-binding fragments, each with a single antigen-binding site and a residual “Fc” fragment. Pepsin treatment yields a F(ab′)₂ fragment that has two antigen-combining sites capable of cross-linking antigen. An “Fv” is the minimum antibody fragment that contains a complete antigen recognition and binding site. In a two-chain Fv species, this region consists of a dimer of one heavy- and one light-chain variable domain in tight, non-covalent association. In a single-chain Fv (scFv) species, one heavy- and one light-chain variable domain are covalently linked by a flexible peptide linker such that the light and heavy chains associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although usually at a lower affinity than the entire binding site.

The Fab fragment also contains the constant domain of the light chain and the first constant domain (C_{H}1) of the heavy chain. Fab fragments differ from Fab′ fragments by the addition of a few residues at the carboxy terminus of the heavy-chain C_{H}1 domain including one or more cysteines from the antibody hinge region. Fab′-SH is the designation herein for Fab′ in which the cysteine residue(s) of the constant domains bear a free thiol group. F(ab)₂ antibody fragments originally were produced as pairs of Fab′ fragments that have hinge cysteines between them. Other chemical couplings of antibody fragments are also suitable. Methods for producing the various fragments from monoclonal Abs include, e.g., Plückthun, 1992, Immunol. Rev. 130:152-188.

“Fv” refers to an antibody fragment which contains a complete antigen-recognition and antigen-binding site. This region consists of a dimer of one heavy chain and one light chain variable domain in tight, non-covalent association. It is in this configuration that the three CDRs of each variable domain interact to define an antigen-binding site on the surface of the V_{H}-V_{L} dimer. Collectively, a combination of one or more of the CDRs from each of the V_{H} and V_{L} chains confer antigen-binding specificity to the antibody. For example, it would be understood that, for example, the CDRH3 and CDRL3 could be sufficient to confer antigen-binding specificity to an antibody when transferred to V_{H} and V_{L} chains of a recipient antibody or antigen-binding fragment thereof and this combination of CDRs can be tested for binding, affinity, etc. using any of the techniques described herein. Even a single variable domain (or half of an Fv comprising only three CDRs specific for an antigen) has the ability to recognize and bind antigen, although likely at a lower affinity than when combined with a second variable domain. Furthermore, although the two domains of a Fv fragment (V_{L} and V_{H}), are coded for by separate genes, they can be joined using recombinant methods by a synthetic linker that enables them to be made as a single protein chain in which the V_{L} and V_{H} regions pair to form monovalent molecules (known as single chain Fv (scFv); Bird et al. (1988) Science 242:423-426; Huston et al. (1988) Proc. Natl. Acad. Sci. USA 85:5879-5883; and Osbourn et al. (1998) Nat. Biotechnol. 16:778). Such scFvs are also intended to be encompassed within the term “antigen-binding portion” of an antibody. Any V_{H} and V_{L} sequences of specific scFv can be linked to an Fc region cDNA or genomic sequences, in order to generate expression vectors encoding complete Ig (e.g., IgG) molecules or other isotypes. V_{H} and V_{L} can also be used in the generation of Fab, Fv or other fragments of Igs using either protein chemistry or recombinant DNA technology.

“Single-chain Fv” or “sFv” antibody fragments comprise the V_{H} and V_{L} domains of an antibody, wherein these domains are present in a single polypeptide chain. In some embodiments, the Fv polypeptide further comprises a polypeptide linker between the V_{H} and V_{L} domains which enables the sFv to form the desired structure for antigen binding. For a review of sFvs see, e.g., Pluckthun in The Pharmacology of Monoclonal Antibodies, Vol. 113, Rosenburg and Moore eds. Springer-Verlag, New York, pp. 269-315 (1994).

The term “Avimer™” refers to a class of therapeutic proteins of human origin, which are unrelated to antibodies and antibody fragments, and are composed of several modular and reusable binding domains, referred to as A-domains (also referred to as class A module, complement type repeat, or LDL-receptor class A domain). They were developed from human extracellular receptor domains by in vitro exon shuffling and phage display (Silverman et al., 2005, Nat. Biotechnol. 23:1493-1494; Silverman et al., 2006, Nat. Biotechnol. 24:220). The resulting proteins can contain multiple independent binding domains that can exhibit improved affinity (in some cases, sub-nanomolar) and specificity compared with single-epitope binding proteins. See, for example, U.S. Patent Application Publ. Nos. 2005/0221384, 2005/0164301, 2005/0053973 and 2005/0089932, 2005/0048512, and 2004/0175756, each of which is hereby incorporated by reference herein in its entirety.

Each of the known 217 human A-domains comprises ˜35 amino acids (˜4 kDa); and domains are separated by linkers that average five amino acids in length. Native A-domains fold quickly and efficiently to a uniform, stable structure mediated primarily by calcium binding and disulfide formation. A conserved scaffold motif of only 12 amino acids is required for this common structure. The end result is a single protein chain containing multiple domains, each of which represents a separate function. Each domain of the proteins specifically binds independently and the energetic contributions of each domain are additive. These proteins were called “Avimer™” from avidity multimers.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444 6448 (1993).

Antigen-binding polypeptides also include heavy chain dimers such as, for example, antibodies from camelids and sharks. Camelid and shark antibodies comprise a homodimeric pair of two chains of V-like and C-like domains (neither has a light chain). Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called V_{HH} domains. Shark Ig-NARs comprise a homodimer of one variable domain (termed a V-NAR domain) and five C-like constant domains (C-NAR domains). In camelids, the diversity of antibody repertoire is determined by the CDRs 1, 2, and 3 in the V_{H} or V_{HH} regions. The CDR3 in the camel V_{HH} region is characterized by its relatively long length, averaging 16 amino acids (Muyldennans at al., 1994, Protein Engineering 7(9): 1129). This is in contrast to CDR3 regions of antibodies of many other species. For example, the CDR3 of mouse V_{H} has an average of 9 amino acids. Libraries of camelid-derived antibody variable regions, which maintain the in vivo diversity of the variable regions of a camelid, can be made by, for example, the methods disclosed in U.S. Patent Application Ser. No. 20050037421.

The term “monoclonal antibody” refers to an antibody obtained from a population of substantially homogeneous antibodies, i.e., the individual antibodies comprising the population are identical except for possible naturally occurring mutations that are present in minor amounts. In some embodiments, monoclonal antibodies are made, for example, by the hybridoma method first described by Köhler and Milstein (1975) Nature 256:495, or are made by recombinant methods, e.g., as described in U.S. Pat. No. 4,816,567. In some embodiments, monoclonal antibodies are isolated from phage antibody libraries using the techniques described in Clackson et al., Nature 352:624-628 (1991), as well as in Marks et al., J. Mol. Biol. 222:581-597 (1991).

The antibodies herein include monoclonal, polyclonal, recombinant, chimeric, humanized, bi-specific, grafted, human, and fragments thereof including antibodies altered by any means to be less immunogenic in humans. Thus, for example, the monoclonal antibodies and fragments, etc., herein include “chimeric” antibodies and “humanized” antibodies. In general, chimeric antibodies include a portion of the heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity (U.S. Pat. No. 4,816,567); Morrison et al. Proc. Natl. Acad. Sci. 81:6851-6855 (1984). For example, in some embodiments, a chimeric antibody contains variable regions derived from a mouse and constant regions derived from human in which the constant region contains sequences homologous to both human IgG2 and human IgG4.

“Humanized” forms of non-human (e.g., murine) antibodies or fragments are chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. Humanized antibodies include, grafted antibodies or CDR grafted antibodies wherein part or all of the amino acid sequence of one or more complementarity determining regions (CDRs) derived from a non-human animal antibody is grafted to an appropriate position of a human antibody while maintaining the desired binding specificity and/or affinity of the original non-human antibody. In some embodiments, corresponding non-human residues replace Fv framework residues of the human immunoglobulin. In some embodiments humanized antibodies comprise residues that are found neither in the recipient antibody nor in the imported CDR or framework sequences. These modifications are made to further refine and optimize antibody performance. In some embodiments, the humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. For further details, see, e.g.: Jones et al., Nature 321: 522-525 (1986); Reichmann et al., Nature 332: 323-329 (1988) and Presta, Curr. Op. Struct. Biol. 2: 593-596 (1992).

As used herein, the term “affinity” refers to the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution.

The phrase “specifically binds” when referring to the interaction between an antibody or other binding molecule and a protein or polypeptide or epitope, typically refers to an antibody or other binding molecule that recognizes and detectably specifically binds with high affinity to the target of interest. Preferably, under designated or physiological conditions, the specified antibodies or binding molecules bind to a particular polypeptide, protein or epitope yet does not bind in a significant or undesirable amount to other molecules present in a sample. In other words the specified antibody or binding molecule does not undesirably cross-react with non-target antigens and/or epitopes. A variety of immunoassay formats are used to select antibodies or other binding molecule that are immunoreactive with a particular polypeptide and have a desired specificity. For example, solid-phase ELISA immunoassays, BIAcore (Surface Plasmon Resonance), flow cytometry and radioimmunoassays are used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that are used to determine or assess immunoreactivity and specificity.

“Selective binding,” “selectivity”, and the like refer the preference of an antibody to interact with one molecule as compared to another. Preferably, interactions between antibodies, particularly modulators, and proteins are both specific and selective. Note that in some embodiments an antibody is designed to “specifically bind” and “selectively bind” two distinct, yet similar targets without binding to other undesirable targets.

An “epitope” or “binding site” is an amino acid sequence or sequences that are “preferentially bound” or “specifically bound” by an antibody or antigen-binding fragment thereof. An epitope can be a linear peptide sequence (i.e., “continuous”) or can be composed of noncontiguous amino acid sequences (i.e., “conformational” or “discontinuous”). Epitopes recognized by an antibody or antigen-binding fragment thereof described herein can be determined by peptide mapping and sequence analysis techniques well known to one of skill in the art.

The terms “polypeptide,” “peptide” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins (i.e., antigens), wherein the amino acid residues are linked by covalent peptide bonds.

The terms “isolated” and “purified” refer to a material that is substantially or essentially removed from or concentrated in its natural environment. For example, an isolated nucleic acid is one that is separated from at least some of the nucleic acids that normally flank it or other nucleic acids or components (proteins, lipids, etc.) in a sample. In another example, a polypeptide is purified if it is substantially removed from or concentrated in its natural environment. Methods for purification and isolation of nucleic acids and proteins are documented methodologies. For example, antibodies can be isolated and purified from the culture supernatant or ascites mentioned above by saturated ammonium sulfate precipitation, euglobulin precipitation method, caproic acid method, caprylic acid method, ion exchange chromatography (DEAF or DE52), or affinity chromatography using anti-Ig column or a protein A, G or L column. Embodiments of “substantially” include at least 20%, at least 40%, at least 50%, at least 75%, at least 85%, at least 90%, at least 95%, or at least 99%.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer stem cell. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering,” “administration,” and the like, as used herein, refer to the methods that are used to enable delivery of agents or compositions to the desired site of biological action. These methods include, but are not limited to oral routes, intraduodenal mutes, parenteral injection (including intravenous, subcutaneous, intraperitoneal, intramuscular, intravascular or infusion), topical and rectal administration. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

### Macrophage Migration Inhibitory Factor (MIF)

In some embodiments, a method and/or composition disclosed herein inhibits (partially or fully) the activity of MIF. In certain instances, MIF is a pro-inflammatory cytokine. In certain instances, it is secreted by activated immune cells (e.g. a lymphocyte (T-cell)) in response to an infection, inflammation, or tissue injury. In certain instances, MIF is secreted by the anterior pituitary gland upon stimulation of the hypothalamic-pituitary-adrenal axis. In certain instances, MIF is secreted together with insulin from the pancreatic beta-cells and acts as an autocrine factor to stimulate insulin release. In certain instances, MIF is a ligand for the receptors CXCR2, CXCR4, and CD74. In some embodiments, a method and/or composition disclosed herein inhibits (partially or fully) the activity of CXCR2 CXCR4, and/or CD74.

In certain instances, MIF induces chemotaxis in nearby leukocytes (e.g. lymphocytes, granulocytes, monocytes/macrophages, and TH-17 cells) along a MIF gradient. In some embodiments, a method and/or composition disclosed herein prevents chemotaxis along a MIF gradient, or reduces chemotaxis along a MIF gradient. In certain instances, MIF induces the chemotaxis of a leukocyte (e.g. lymphocytes, granulocytes, monocytes/macrophages, and TH-17 cells) to the site of an infection, inflammation or tissue injury. In some embodiments, a method and/or composition disclosed herein prevents or decreases the chemotaxis of a leukocyte to the site of an infection, inflammation or tissue injury. In certain instances, the chemotaxis of a leukocyte (e.g. lymphocytes, granulocytes, monocytes/macrophages, and TH-17 cells) along a MIF gradient results in inflammation at the site of infection, inflammation, or tissue injury. In some embodiments, a method and/or composition disclosed herein treats inflammation at the site of infection, inflammation, or tissue injury. In certain instances, the chemotaxis of monocytes along a RANTES gradient results in monocyte arrest (i.e., the deposition of monocytes on epithelium) at the site of injury or inflammation. In some embodiments, a method and/or composition disclosed herein prevents or decreases monocyte arrest at the site of injury or inflammation. In some embodiments, a method and/or composition disclosed herein inhibits treats a lymphocyte mediated disorder. In some embodiments, a method and/or composition disclosed herein treats a granulocyte mediated disorder. In some embodiments, a method and/or composition disclosed herein treats a macrophage mediated disorder. In some embodiments, a method and/or composition disclosed herein treats a Th-17 mediated disorder. In some embodiments, a method and/or composition disclosed herein treats a pancreatic beta-cell mediated disorder.

In certain instances, MIF is inducible by glucocorticoids, a mechanism implicated in an acceleration of atherosclerosis associated with many diseases requiring glucocorticoid therapy. Thus, in some embodiments, the compositions and methods described herein inhibit the induction of MIF expression by glucocorticoids.

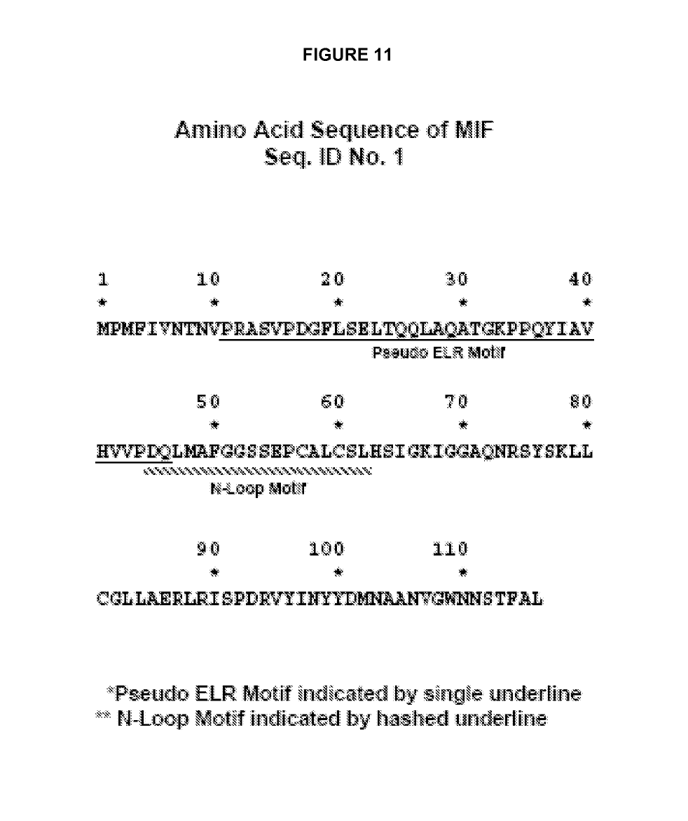

In certain instances, a human MIF polypeptide is encoded by a nucleotide sequence located on chromosome 22 at the cytogenic band 22q11.23. In certain instances, a MIF protein is a 12.3 kDa protein. In certain instances, a MIF protein is a homotrimer comprising three polypeptides of 115 amino acids. In certain instances, a MIF protein comprises a pseudo-ELR motif that mimics the ELR motif found in chemokines. In certain instances, the pseudo-ELR motif comprises two nonadjacent but adequately spaced residues (Arg12 and Asp45 & see FIG. 11). In some embodiments the pseudo-ELR motif comprises the amino acid sequence from amino acid 12 to amino acid 45 (such numbering includes the first methionine residue). This is equivalent to a pseudo-ELR motif from amino acid 11 to amino acid 44 in which the first methionine residue is not counted (in such instances, the pseudo-ELR motif comprises Arg 11 and Asp 44). In some embodiments, a method and/or composition disclosed herein treats a MIF-mediated disorder by inhibiting binding of the pseudo-ELR motif to CXCR2 and/or CXCR4.

In certain instances, a MIF protein comprises a 10- to 20-residue N-terminal Loop motif (N-loop). In certain instances, a MIF N-loop mediates binding to a CXCR2 and/or CXCR4 receptor. In certain instances, the N-loop motif of MIF comprises the sequential residues (47-56) of MIF (i.e. L47 M48 A49 F50 G51 G52 S53 S54 E55 P56; see FIG. 11). In certain instances, the N-loop motif of MIF comprises amino acids 45-60. In certain instances, the N-loop motif of MIF comprises amino acids 44-61. In certain instances, the N-loop motif of MIF comprises amino acids 43-62. In certain instances, the N-loop motif of MIF comprises amino acids 42-63. In certain instances, the N-loop motif of MIF comprises amino acids 41-64. In certain instances, the N-loop motif of MIF comprises amino acids 40-65. In certain instances, the N-loop motif of MIF comprises amino acids 46-59. In certain instances, the N-loop motif of MIF comprises amino acids 47-59. In certain instances, the N-loop motif of MIF comprises amino acids 48-59. In certain instances, the N-loop motif of MIF comprises amino acids 50-59. In certain instances, the N-loop motif of MIF comprises amino acids 47-58. In certain instances, the N-loop motif of MIF comprises amino acids 47-57. In certain instances, the N-loop motif of MIF comprises amino acids 47-56. In certain instances, the N-loop motif of MIF comprises amino acids 48-58. In some embodiments the N-Loop motif comprises amino acids 48-57. In some embodiments, a method and/or composition disclosed herein treats a MIF-mediated disorder by inhibiting binding of the N-loop motif to CXCR2 and/or CXCR4.

In some embodiments, a method and/or composition disclosed herein treats a MIF-mediated disorder by inhibiting (1) binding of the N-loop motif to CXCR2 and/or CXCR4; and (2) binding of the pseudo-ELR motif to CXCR2 and/or CXCR4.

In certain instances, CD74 activates G-protein coupled receptors (GPCRs), activates CXCR2, and/or associates with these molecules to form signaling complex. Thus, in some embodiments, a method and/or composition disclosed herein treats a MIF-mediated disorder by inhibiting the activation GPCRs or CXCR2 by CD74.

In certain instances, MIF is expressed by endothelial cells, SMCs, mononuclear cells, and/or macrophages following arterial injury. In some embodiments, a method and/or composition disclosed herein inhibits the expression of MIF by endothelial cells, SMCs, mononuclear cells, and/or macrophages following arterial injury. In certain instances, MIF is expressed by endothelial cells, SMCs, mononuclear cells, macrophages following exposure to oxidized low-density lipoprotein (oxLDL), CD40 ligand, angiotensin II, or combinations thereof. In some embodiments, a method and/or composition disclosed herein inhibits the expression of MIF by endothelial cells, SMCs, mononuclear cells, and/or macrophages following exposure to oxidized low-density lipoprotein, CD40 ligand, angiotensin II, or combinations thereof.

In certain instances, MIF induces expression of CCL2, TNF, and/or ICAM-1 in endothelial cells. In some embodiments, a method and/or composition disclosed herein inhibits the MIF-induced expression of CCL2, TNF, and/or ICAM-1 in endothelial cells.

In certain instances, MIF induces expression of MMPs and cathepsins in SMCs. In some embodiments, a method and/or composition disclosed herein inhibits the MIF-induced expression of MMPs and cathepsins in SMCs.

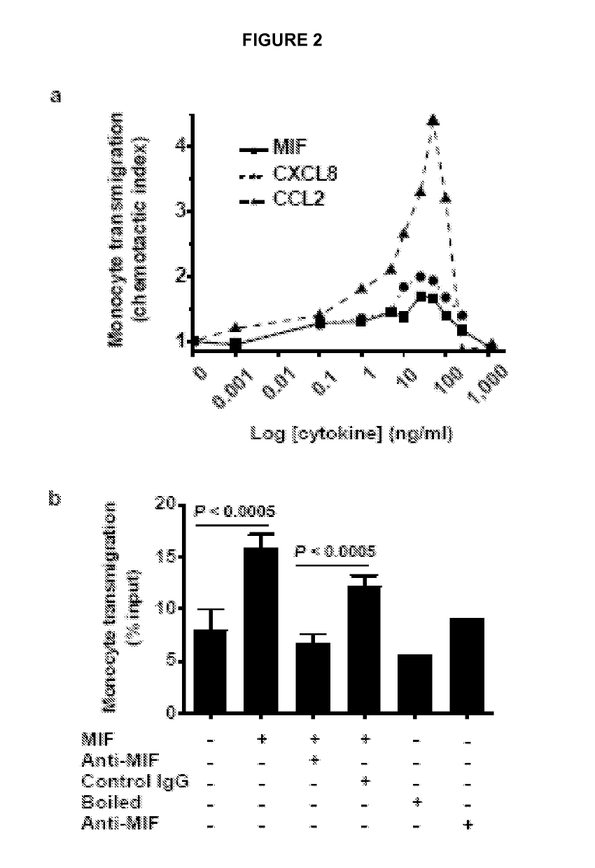

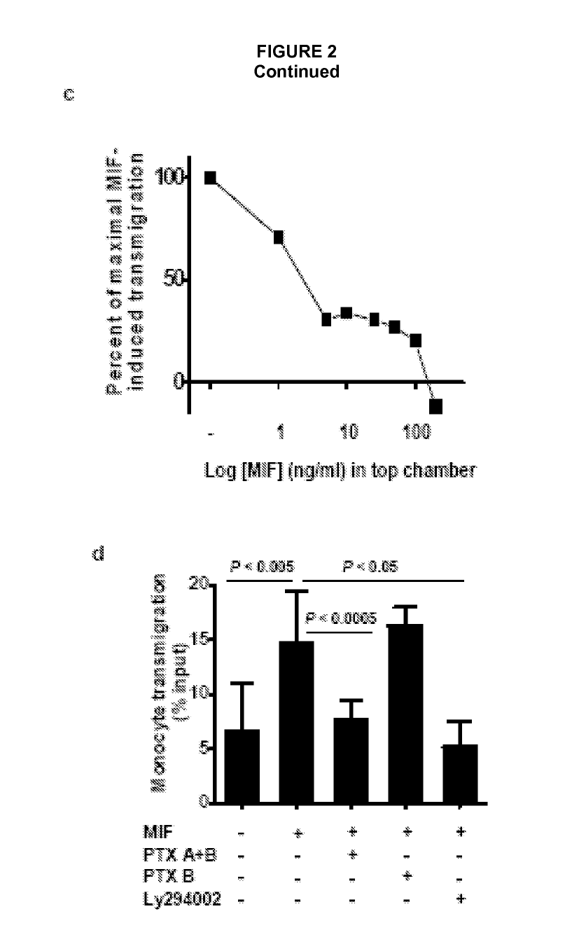

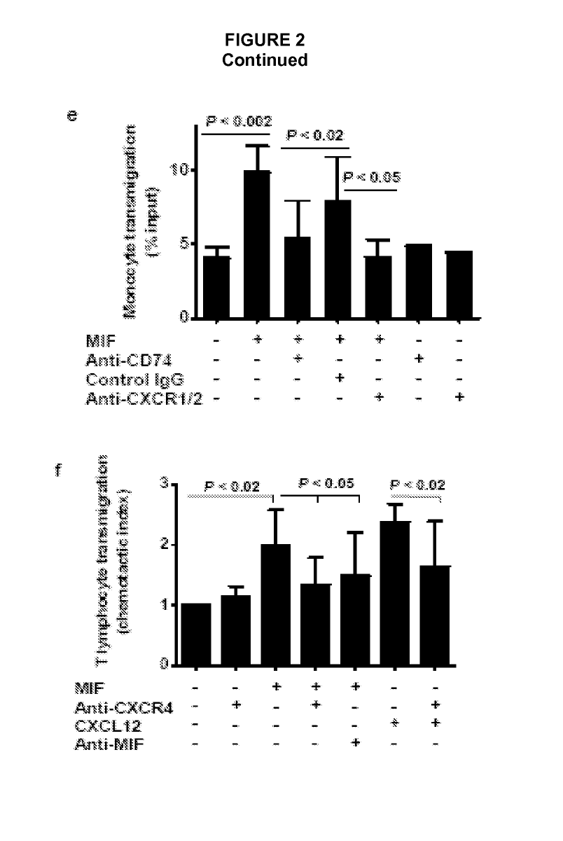

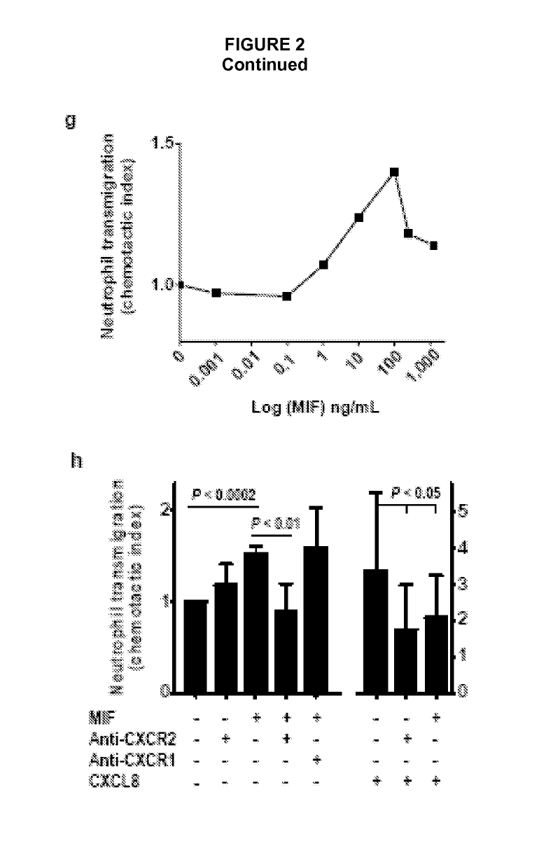

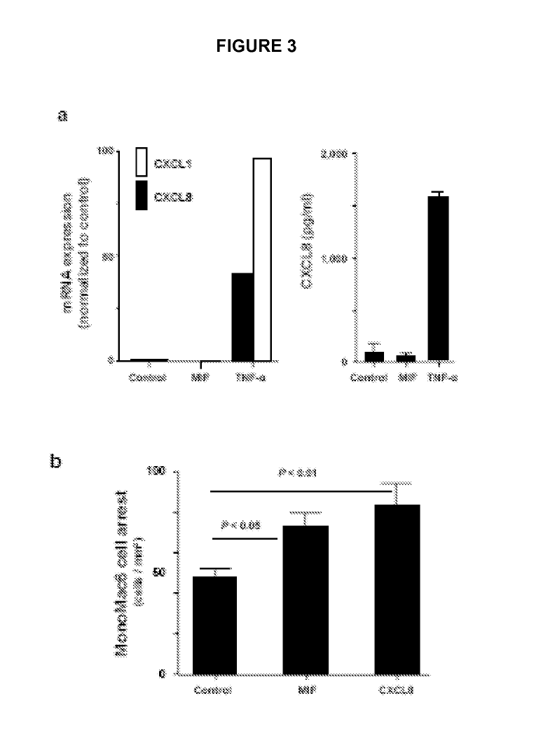

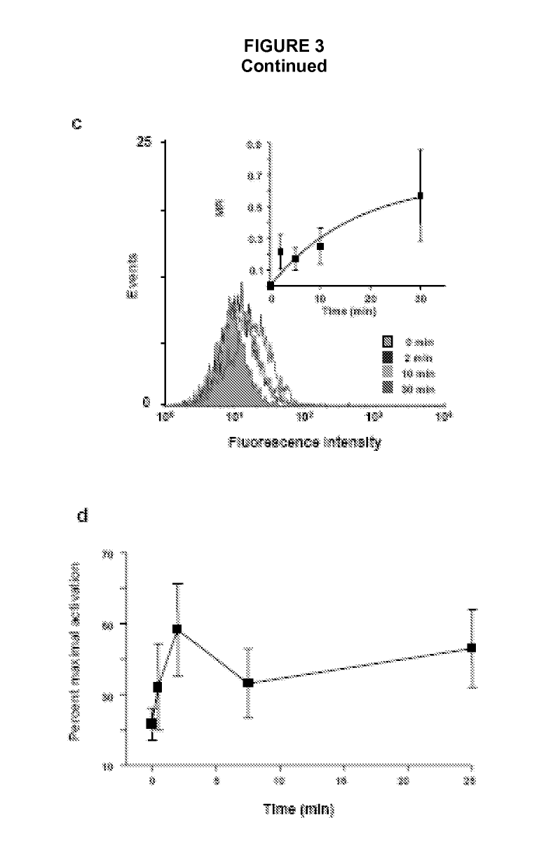

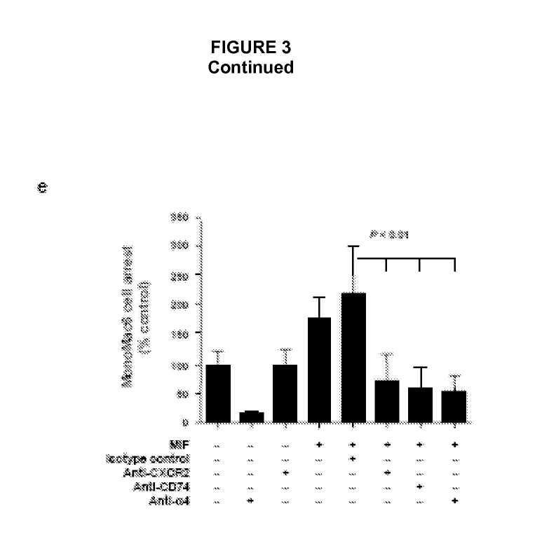

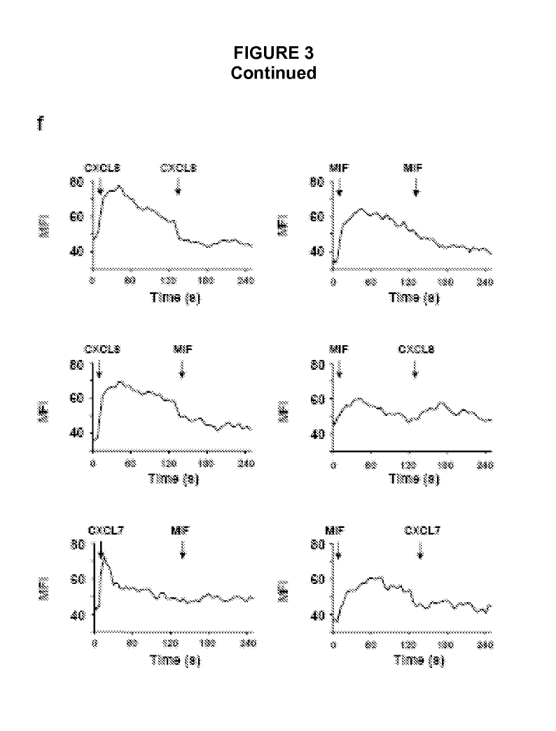

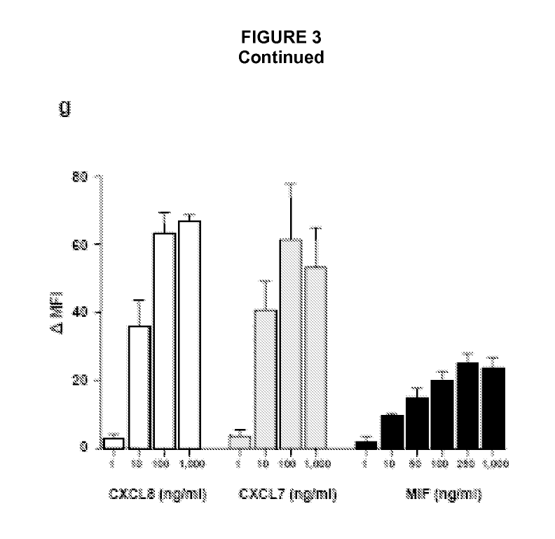

In certain instances, MIF triggers a calcium influx through CXCR2 or CXCR4, induces a rapid activation of integrins, induces MAPK activation, and mediates the Gαi- and integrin dependent arrest and the chemotaxis of monocytes and T cells (FIGS. 2 and 3). Thus, In some embodiments, a method and/or composition disclosed herein inhibits calcium influx in monocytes and/or T cells, inhibit activation of MAPK, inhibit activation of integrins, inhibit Gαi- and integrin dependent arrest of monocytes and T cells, or combinations thereof.

In some embodiments, the methods described herein comprise an anti-CXCR2 antibody; an anti-CXCR4 antibody; an anti-MIF antibody; or combinations thereof. In some embodiments, an antibody disclosed herein inhibits the binding of MIF to CXCR2 and/or CXCR4 by binding to a pseudo-ELR motif of MIF. In some embodiments, an antibody disclosed herein inhibits the binding of MIF to CXCR2 and/or CXCR4 by binding to an N-loop motif of MIF. In some embodiments, an antibody disclosed herein inhibits the binding of MIF to CXCR2 and/or CXCR4 by simultaneously binding to both an N-loop motif AND a pseudo-ELR motif of MIF. In some embodiments, an antibody disclosed herein is an anti-MIF antibody.

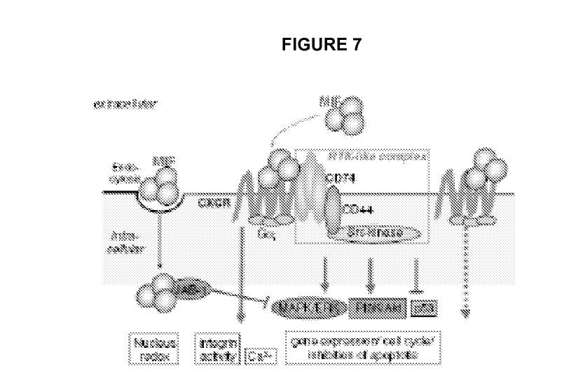

In certain instances, monocyte recruitment induced by MIF involves the MIF-binding protein CD74. In certain instances, the MIF-binding protein CD74 induces calcium influx, mitogen-activated protein kinase (MAPK) activation, or Gαi-dependent integrin activation (FIG. 7). In some embodiments the present invention comprises a method of inhibiting MIF mediated MAPK kinase activation in an individual in need thereof. In some embodiments the present invention comprises a method of inhibiting MIF mediated Gαi-dependent integrin activation in an individual in need thereof.

In certain instances, MIF-induced signaling via CD74 involves CD44 and Src kinases. In some embodiments, a method and/or composition disclosed herein inhibits CD74-mediated Src kinase activation.

In certain instances, MIF taken up by endocytosis interacts directly with JAB-1. In some embodiments, a method and/or composition disclosed herein inhibits endocytosis of MIF.

In certain instances, arrestins facilitate the recruitment of G protein-coupled receptors to the clathrin-coated vesicles that mediate MIF internalization. Thus, in some embodiments, a method and/or composition disclosed herein further comprises an arrestin antagonist. Examples of agents that inhibit arrestin binding to a GPCR comprise carvedilol, isoprenaline, isoproterenol, formoterol, cimeterol, clenbuterol, L-epinepherine, spinophilin and salmeterol.

In certain instances, ubiquitylation of MIF results in (either partially or fully) the rapid internalization and subsequent degradation of MIF. Thus, in some embodiments, a method and/or composition disclosed herein further comprises inhibiting ubiquitylation of MIF. Examples of agents that inhibit ubiquitylation include, but are not limited to, PYR-41 and related pyrazones.

In certain instances, MIF enters cells using clathrin-mediated endocytosis. Thus, in some embodiments, a method and/or composition disclosed herein further comprises inhibiting clathrin-mediated endocytosis of MIF.

In certain instances, MIF negatively regulates MAPK signaling or modulates cell functions by regulating cellular redox homeostasis through JAB-1. In certain instances, MIF down-regulates p53 expression. In certain instances, MIF downregulation of p53 expression results in inhibition of apoptosis and prolonged survival of macrophages. Thus, in some embodiments, a method and/or composition disclosed herein inhibits MIF-modulated survival of macrophages.

In certain instances, MIF induces MMP-1 and MMP-9 in vulnerable plaques. In certain instances, the induction of MMP-1 and MMP-9 in vulnerable plaques results in (either partially or fully) collagen degradation, a weakening of the fibrous cap, and plaque destabilization. In some embodiments, a method and/or composition disclosed herein inhibits (either partially or fully) collagen degradation, weakening of the fibrous cap, and plaque destabilization.

### Inhibitors of MIF Signaling Through CXCR2 and CXCR4

Disclosed herein, in certain embodiments, are methods of inhibiting MIF signaling through CXCR2 and CXCR4. In some embodiments, MIF signaling through CXCR2 and CXCR4 is inhibited by occupying the MIF binding domain of CXCR2 and CXCR4 (i.e., the GPCR antagonist approach) with an antibody. In some embodiments MIF signaling through CXCR2 and CXCR4 is inhibited by occupying, masking, or otherwise disrupting domains on MIF (i.e., the cytokine inhibitor approach). In some embodiments, MIF signaling through CXCR2 and CXCR4 is inhibited by an antibody occupying, masking, or otherwise disrupting domains on MIF and thereby disrupting the binding of CXCR2 and/or CXCR4 to MIF. In some embodiments, MIF signaling through CXCR2 and CXCR4 is inhibited by an antibody occupying, masking, or otherwise disrupting domains on MIF and thereby disrupting MIF trimerization. In certain instances, occupying, masking, or otherwise disrupting domains on MIF does not affect CXCR2 and CXCR4 signaling mediated by other agonists/ligands (e.g., IL-8/CXCL8, GRObeta/CXCL2 and/or Stromal Cell-Derived Factor-1a (SDF-1a)/CXCL12).

### MIF Domain Disrupting Agents

In some embodiments MIF signaling through CXCR2 and CXCR4 is inhibited by occupying, masking, or otherwise disrupting domains on MIF (e.g., the N-loop and/or the pseudo-ELR motif). In some embodiments, MIF signaling through CXCR2 and CXCR4 is inhibited by an antibody occupying, masking, or otherwise disrupting domains on MIF and thereby disrupting the binding of CXCR2 and/or CXCR4 to MIF. In some embodiments, an antibody inhibits (i) MIF binding to CXCR2 and CXCR4 and/or (ii) MIF-activation of CXCR2 and CXCR4; or (iii) any combination of (i) and (ii). In certain instances, occupying, masking, or otherwise disrupting domains on MIF does not affect CXCR2 and CXCR4 signaling mediated by other agonists/ligands (e.g., IL-8/CXCL8, GRObeta/CXCL2 and/or Stromal Cell-Derived Factor-1a (SDF-1a)/CXCL12).

In certain instances, the N-terminal extracellular domain as well as the first and/or second extracellular loop are mediators of ligand binding to MIF. In some embodiments, an antibody inhibits the binding of MIF to CXCR2 and/or CXCR4 by binding to a pseudo-ELR motif of MIF. In some embodiments, an antibody inhibits the binding of MIF to CXCR2 and/or CXCR4 by binding to an N-loop motif of MIF. In some embodiments, an antibody modulates critical residues and/or invokes a conformational change in MIF that prevents receptor or substrate interactions. In some embodiments an antibody interferes with motifs relevant for CXCR2 and/or CXCR4 binding and signaling.

### MIF Trimerization Disrupting Agents

Disclosed herein, in certain embodiments, are methods of inhibiting MIF signaling through CXCR2 and CXCR4. In some embodiments MIF signaling through CXCR2 and CXCR4 is inhibited by occupying, masking, or otherwise disrupting domains on MIF. In some embodiments, MIF signaling through CXCR2 and CXCR4 is inhibited by an antibody occupying, masking, or otherwise disrupting domains on MIF and thereby disrupting MIF trimerization. In some embodiments, impairing the ability of a MIF peptide to form a homotrimer disrupts (partially or fully) the ability of MIF to bind to a receptor (e.g., CXCR2, or CXCR4). In certain instances, occupying, masking, or otherwise disrupting domains on MIF does not affect CXCR2 and CXCR4 signaling mediated by other agonists/ligands (e.g., IL-8/CXCL8, GRObeta/CXCL2 and/or Stromal Cell-Derived Factor-1a (SDF-1a)/CXCL12).

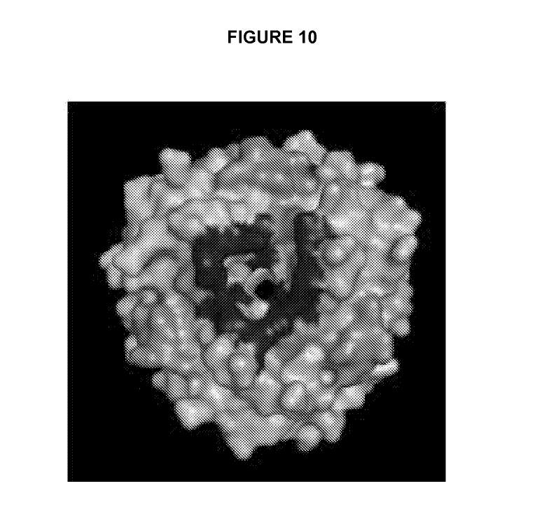

In certain instances, MIF comprises three MIF polypeptide sequences (i.e., a trimer). In certain instances, the pseudo-ELR motifs of each MIF polypeptide form a ring in the trimer. In certain instances, the N-loop motifs of each MIF polypeptide extend outwards from the pseudo-ELR ring (see FIG. 10). In certain instances, disruption of the trimer disrupts the high affinity binding of MIF to its target receptors. In certain instances, residues 38-44 (beta-2 strand) of one subunit interact with residues 48-50 (beta-3 strand) of a second subunit. In certain instances, residues 96-102 (beta-5 strand) of one subunit interact with residues 107-109 (beta-6 strand) of a second subunit. In certain instances, a domain on one subunit formed by N73 R74 S77 K78 C81 interacts with N111 S112 T113 of a second subunit.

In some embodiments, an anti-MIF antibody is derived from and/or specifically binds to any or all of amino acid residues 38-44 (beta-2 strand) of MIF. In some embodiments, an anti-MIF antibody is derived from and/or specifically binds to any or all of amino acid residues 48-50 (beta-3 strand) of MIF. In some embodiments, an anti-MIF antibody is derived from and/or specifically binds to any or all of amino acid residues 96-102 (beta-5 strand) of MIF. In some embodiments, an anti-MIF antibody is derived from and/or specifically binds to any or all of amino acid residues 107-109 (beta-6 strand) of MIF. In some embodiments, an anti-MIF antibody is derived from and/or specifically binds to any or all of amino acid residues N73, R74, S77, K78, and C81 of MIF. In some embodiments, an anti-MIF antibody is derived from and/or specifically binds to any or all of amino acid residues N111, S112, and T113 of MT.

### Antibodies

Disclosed herein, in certain embodiments, is a method of treating a MIF-mediated disorder in an individual in need thereof. In some embodiments, the method comprises administering a therapeutically-effective amount of an anti-CXCR2 antibody; an anti-CXCR4 antibody; an anti-MIF antibody; or combinations thereof. In some embodiments, the methods described herein comprise an anti-CXCR2 antibody. In some embodiments, the methods described herein comprise an anti-CXCR4 antibody. In some embodiments, the methods described herein comprise an anti-MIF antibody.

In some embodiments, the antibody is an antibody that specifically binds to all or part of the pseudo-ELR motif of MIF. In some embodiments, the part of the pseudo-ELR motif of MIF that is bound by the antibody is a part of the pseudo-ELR motif that is exposed or on the outside of a MIF trimer. In some embodiments, the antibody specifically binds to all or a portion of a peptide sequence as follows: PRASVPDGFLSELTQQLAQATGKPPQYIAVHVVPDQ and the corresponding feature/domain of at least one of a MIF monomer or MIF trimer. In some embodiments, the antibody specifically binds to all or a portion of an amino acid sequence from amino acid 11 to amino acid 44 (See Seq ID No. 1) and the corresponding feature/domain of at least one of a MIF monomer or MIF trimer.

In some embodiments, the antibody is an antibody that specifically binds to all or part of the N-loop motif of MIF. In some embodiments, the part of the N-loop motif of MIF that is bound by the antibody is a part of the N-loop motif that is exposed or on the outside of a MIF trimer. In some embodiments, the antibody specifically binds to all or a portion of a peptide sequence as follows: DQLMAFGGSSEPCALCSL and the corresponding feature/domain of at least one of a MIF monomer or MIF trimer. In some embodiments, the antibody specifically binds to all or a portion all or a portion of an amino acid sequence from amino acid 40 to amino acid 65 (See Seq ID No. 1) and the corresponding feature/domain of at least one of a MIF monomer or MIF trimer.

In some embodiments, the antibody is an antibody that specifically binds to all or a portion of the pseudo-ELR motif of MIF and all or a portion of the N-loop motif of MIF. In some embodiments, the parts of the N-loop and pseudo-ELR motifs of MIF that are bound by the antibody are part that are exposed or on the outside of a MIF trimer. In some embodiments, the antibody specifically binds to all or a portion of a peptide sequence as follows: PRASVPDGFLSELTQQLAQATGKPPQYIAVHVVPDQLMAFGGSSEPCALCSL and the corresponding feature/domain of at least one of a MIF monomer or MIF trimer. In some embodiments, the antibody specifically binds to all or a portion all or a portion of an amino acid sequence from amino acid 11 to amino acid 65 (See Seq ID No. 1) and the corresponding feature/domain of at least one of a MIF monomer or MIF timer.

In some embodiments, the antibody specifically binds to the CXCR2 binding domain of MIF.

In some embodiments, the antibody specifically binds to the CXCR4 binding domain of MIF.

In some embodiments, the antibody inhibits the formation of a MIF trimer.

In some embodiments, the antibody is an anti-CD74 antibody. In some embodiments, the antibody inhibits the binding of MT to CD74. In some embodiments, the anti-CD74 antibody is or is derived from M-B741 (Pharmingen).

In some embodiments, the antibody is an anti-Jab-1 antibody. In some embodiments, the antibody inhibits the binding of MIF to JAB-1. In some embodiments, the antibody specifically binds to all or a portion of an amino acid sequence from amino acid 50 to amino acid 65 (See Seq ID No. 1) and the corresponding feature/domain of at least one of a MIF monomer or MIF trimer. In some embodiments, the antibody specifically binds to all or a portion of a peptide sequence as follows: FGGSSEPCALCSLHSI and the corresponding feature/domain of at least one of a MIF monomer or MIF trimer.

In some embodiments, the antibody is an anti-CXCR2 antibody. In some embodiments, the antibody antagonist is a monoclonal antibody. In some embodiments, the antibody antagonist is a polyclonal antibody. In some embodiments, the antibody antagonist is selected from CXCR2 antibody, clone 48311.211; CXCR2 antibody, clone 5E8/CXCR2; CXCR2 antibody, clone 19; or derivatives thereof.

In some embodiments, the antibody is an anti-CXCR4 antibody selected from CXCR4 antibody, clone 701; CXCR4 antibody, clone 708; CXCR4 antibody, clone 716; CXCR4 antibody, clone 717; CXCR4 antibody, clone 718; CXCR4 antibody, clone 12G5; CXCR4 antibody, clone 4G10; or combinations thereof.

In some embodiments, the antibody is an anti-MIF antibody selected from MIF antibody, clone IID.9; MIF antibody, clone IIID.9; MIF antibody, clone XIF7; MIF antibody, clone 131; MIF antibody, clone IV2.2; MIF antibody, clone XI7; MIF antibody, clone XII15.6; MIF antibody, clone XIV15,4; or combinations thereof.

### Production of Monoclonal Antibodies

In some embodiments, monoclonal antibodies (mAb) against a peptide disclosed herein are produced via the use of a hybridoma. In certain instances, a hybridoma is an immortalized antibody producing cell. In some embodiments, a laboratory animal (e.g., a mouse or a rabbit) is inoculated with an antigen. In some embodiments, B-cells from the laboratory animal's spleen are extracted. In some embodiments, a hybridoma is generated by fusing (1) an extracted B-cell with (2) a myeloma cell (i.e., hypoxanthine-guanine-phosphoribosyl transferase negative, immortalized myeloma cells). In some embodiments, the B-cell and the myeloma cells are cultured together and exposed to an agent that renders their cell membranes more permeable (e.g., PEG).

In some embodiments, the culture comprises a plurality of hybridoma, a plurality of myeloma cells, and a plurality of B-cells. In some embodiments, the cells are subjected to culturing conditions that select for hybridoma (e.g., culturing with HAT media).

In some embodiments, an individual hybridoma (i.e., the clone) is isolated and cultured. In some embodiments, the hybridoma is injected into a laboratory animal (e.g., a rabbit or rat). In some embodiments, the hybridoma are cultured in a cell culture.

In some embodiments, the methods described herein comprise a humanized monoclonal antibody. In some embodiments, a humanized monoclonal antibody comprises heavy and light chain constant regions from a human source and variable regions from a murine source.

In some embodiments, humanized immunoglobulins, including humanized antibodies, are constructed by genetic engineering. In some embodiments, humanized immunoglobulins comprise a framework that is identical to the framework of a particular human immunoglobulin chain (i.e., an acceptor or recipient), and three CDRs from a non-human (donor) immunoglobulin chain. In some embodiments, a limited number of amino acids in the framework of a humanized immunoglobulin chain are identified and chosen to be the same as the amino acids at those positions in the donor rather than in the acceptor.

In some embodiments, a framework is used from a particular human immunoglobulin that is homologous to the donor immunoglobulin to be humanized. For example, comparison of the sequence of a mouse heavy (or light) chain variable region against human heavy (or light) variable regions in a data bank (for example, the National Biomedical Research Foundation Protein Identification Resource or the protein sequence database of the National Center for Biotechnology Information—NCBI) shows that the extent of homology to different human regions can vary greatly, for example from about 40% to about 60%, about 70%, about 80%, or higher. By choosing as the acceptor immunoglobulin one of the human heavy chain variable regions that is most homologous to the heavy chain variable region of the donor immunoglobulin, fewer amino acids will be changed in going from the donor immunoglobulin to the humanized immunoglobulin. By choosing as the acceptor immunoglobulin one of the human light chain variable regions that is most homologous to the light chain variable region of the donor immunoglobulin, fewer amino acids will be changed in going from the donor immunoglobulin to the humanized immunoglobulin.

In some embodiments, a humanized immunoglobulin comprises light and heavy chains from the same human antibody as acceptor sequences. In some embodiments, a humanized immunoglobulin comprises light and heavy chains from different human antibody germline sequences as acceptor sequences; when such combinations are used, one can readily determine whether the VH and VL bind an epitope of interest using conventional assays (e.g., an ELISA). In some embodiments, the human antibody will be chosen in which the light and heavy chain variable regions sequences, taken together, are overall most homologous to the donor light and heavy chain variable region sequences. In some embodiments, higher affinity is achieved by selecting a small number of amino acids in the framework of the humanized immunoglobulin chain to be the same as the amino acids at those positions in the donor rather than in the acceptor.

Any suitable method of modifying a framework region is contemplated herein. In some embodiments, the relevant framework amino acids to change are selected based on differences in amino acid framework residues between the donor and acceptor molecules. In some embodiments, the amino acid positions to change are residues known to be important or to contribute to CDR conformation (e.g., canonical framework residues are important for CDR conformation and/or structure). In some embodiments, the relevant framework amino acids to change are selected based on frequency of an amino acid residue at a particular framework position (e.g., comparison of the selected framework with other framework sequences within its subfamily can reveal residues that occur at minor frequencies at a particular position or positions). In some embodiments, the relevant framework amino acids to change are selected based on proximity to a CDR. In some embodiments, the relevant framework amino acids to change are selected based on known or predicted proximity to the antigen-CDR interface or predicted to modulate CDR activity. In some embodiments, the relevant framework amino acids to change are framework residues that are known to, or predicted to, form contacts between the heavy (VH) and light (VL) chain variable region interface. In some embodiments, the relevant framework amino acids to change are framework residues that are inaccessible to solvent.

In some embodiments, amino acid changes at some or all of the selected positions are incorporated into encoding nucleic acids for the acceptor variable region framework and donor CDRs. In some embodiments, altered framework or CDR sequences are individually made and tested, or are sequentially or simultaneously combined and tested.

In some embodiments, the variability at any or all of the altered positions is from a few to a plurality of different amino acid residues, including all twenty naturally occurring amino acids or functional equivalents and analogues thereof. In some embodiments, non-naturally occurring amino acids are considered.

In some embodiments, the humanized antibody sequence is cloned into a vector. In some embodiments, any suitable vector is used. In some embodiments, the vector is a plasmid, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference for such disclosure.

In some embodiments, any suitable host cell is transformed with the vector expressing the humanized antibody sequence. In some embodiments, the host cell is bacteria, mammalian cells, yeast and baculovirus systems. The expression of antibodies and antibody fragments in prokaryotic cells such as E. coli is well established in the art. For a review, see for example Plückthun, A. Bio/Technology 9: 545-551 (1991). Expression in eukaryotic cells in culture is also available to those skilled in the art as an option for production of the antibodies and antigen-binding fragments described herein, see for recent reviews, for example Raff, M. E. (1993) Curr. Opinion Biotech. 4: 573-576; Trill J. J. et al. (1995) Curr. Opinion Biotech 6: 553-560, each of which is which is incorporated herein by reference for such disclosure.

In some embodiments, a mammalian expression system is used. In some embodiments, the mammalian expression system is dehydrofolate reductase deficient (“dhfr-”) Chinese hamster ovary cells. In some embodiments, dhfr-CHO cells are transfected with an expression vector containing a functional DHFR gene, together with a gene that encodes a desired humanized antibody.

In some embodiments, DNA is transformed by any suitable method. For eukaryotic cells, suitable techniques include, for example, calcium phosphate transfection, DEAE Dextran, electroporation, liposome-mediated transfection and transduction using retrovirus or other virus, e.g., vaccinia or, for insect cells, baculovirus. For bacterial cells, suitable techniques include, for example, calcium chloride transformation, electroporation and transfection using bacteriophage.

In some embodiments, a DNA sequence encoding an antibody or antigen-binding fragment thereof is prepared synthetically rather than cloned. In some embodiments, the DNA sequence is designed with the appropriate codons for the antibody or antigen-binding fragment amino acid sequence. In general, one will select preferred codons for the intended host if the sequence will be used for expression. In some embodiments, the complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984), each of which is which is incorporated herein by reference for such disclosure.

### Cell Lines

Disclosed herein, in certain embodiments, is a cell line that expresses a recombinant human CXCR4 plus human CD74. In some embodiments, the cell line that expresses a recombinant human CXCR4 plus human CD74 is a human cell line (e.g., HEK293). In some embodiments, the cell line that expresses a recombinant human CXCR4 plus human CD74 is a non-human cell line (e.g., CHO).

### Inflammation

In some embodiments, a method and/or composition described herein treats a MIF-mediated disorder. In some embodiments, a method and/or composition described herein treats inflammation (e.g., acute or chronic). In some embodiments, a method and/or composition described herein treats inflammation resulting from (either partially or fully) an infection. In some embodiments, a method and/or composition described herein treats inflammation resulting from (either partially or fully) damage to a tissue (e.g., by a burn, by frostbite, by exposure to a cytotoxic agent, or by trauma). In some embodiments, a method and/or composition described herein treats inflammation resulting from (either partially or fully) an autoimmune disorder. In some embodiments, a method and/or composition described herein treats inflammation resulting from (either partially or fully) the presence of a foreign body (e.g., a splinter). In some embodiments, a method and/or composition described herein treats inflammation resulting from exposure to a toxin and/or chemical irritant.

As used herein, “acute inflammation” refers to inflammation characterized in that it develops over the course of a few minutes to a few hours, and ceases once the stimulus has been removed (e.g., an infectious agent has been killed by an immune response or administration of a therapeutic agent, a foreign body has been removed by an immune response or extraction, or damaged tissue has healed). The short duration of acute inflammation results from the short half-lives of most inflammatory mediators.

In certain instances, acute inflammation begins with the activation of leukocytes (e.g., dendritic cells, endothelial cells and mastocytes). In certain instances, the leukocytes release inflammatory mediators (e.g., histamines, proteoglycans, serine proteases, eicosanoids, and cytokines). In certain instances, inflammatory mediators result in (either partially or fully) the symptoms associated with inflammation. For example, in certain instances an inflammatory mediator dilates post capillary venules, and increases blood vessel permeability. In certain instances, the increased blood flow that follows vasodilation results in (either partially or fully) rubor and calor. In certain instances, increased permeability of the blood vessels results in an exudation of plasma into the tissue leading to edema. In certain instances, the latter allows leukocytes to migrate along a chemotactic gradient to the site of the inflammatory stimulant. Further, in certain instances, structural changes to blood vessels (e.g., capillaries and venules) occur. In certain instances, the structural changes are induced (either partially or fully) by monocytes and/or macrophages. In certain instances, the structural changes include, but are not limited to, remodeling of vessels, and angiogenesis. In certain instances, angiogenesis contributes to the maintenance of chronic inflammation by allowing for increased transport of leukocytes. Additionally, in certain instances, histamines and bradykinin irritate nerve endings leading to itching and/or pain.

In certain instances, chronic inflammation results from the presence of a persistent stimulant (e.g., persistent acute inflammation, bacterial infection (e.g., by Mycobacterium tuberculosis), prolonged exposure to chemical agents (e.g., silica, or tobacco smoke) and autoimmune reactions (e.g., rheumatoid arthritis)). In certain instances, the persistent stimulant results in continuous inflammation (e.g., due to the continuous recruitment of monocytes, and the proliferation of macrophages). In certain instances, the continuous inflammation further damages tissues which results in the additional recruitment of mononuclear cells thus maintaining and exacerbating the inflammation. In certain instances, physiological responses to inflammation further include angiogenesis and fibrosis.

In some embodiments, a method and/or composition described herein treats a disorder associated with inflammation (i.e., inflammatory disorders). Inflammatory disorders include, but are not limited to, Atherosclerosis; Abdominal aortic aneurysm; Acute disseminated encephalomyelitis; Moyamoya disease; Takayasu disease; Acute coronary syndrome; Cardiac-allograft vasculopathy; Pulmonary inflammation; Acute respiratory distress syndrome; Pulmonary fibrosis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Septic shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Neointimal hyperplasia, Myocardial infarction; Stroke; Organ transplant rejection; or combinations thereof.

### Atherosclerosis

In some embodiments, a method and/or composition described herein treats atherosclerosis. As used herein, “atherosclerosis” means inflammation of an arterial wall and includes all phases of atherogenesis (e.g., lipid deposition, intima-media thickening, and subintimal infiltration with monocytes) and all atherosclerotic lesions (e.g., Type I lesions to Type VIII lesions). In certain instance, atherosclerosis results from (partially or fully) the accumulation of macrophages. In some embodiments, the methods and compositions described herein prevent the accumulation of macrophages, decrease the number of accumulated macrophages, and/or decrease the rate at which macrophages accumulate. In certain instances, atherosclerosis results from (partially or fully) the presence of oxidized LDL. In certain instances, oxidized LDL damages an arterial wall. In some embodiments, the methods and compositions described herein prevent oxidized LDL-induced damage to an arterial wall, decrease the portion of an arterial wall damaged by oxidized LDL, decrease the severity of the damage to an arterial wall, and/or decrease the rate at which an arterial wall is damaged by oxidized LDL. In certain instances, monocytes respond to (i.e., follow a chemotactic gradient to) the damaged arterial wall. In certain instances, the monocytes differentiate macrophages. In certain instances, macrophages endocytose the oxidized-LDL (cells such as macrophages with endocytosed LDL are called “foam cells”). In some embodiments, the methods and compositions described herein prevent the formation of foam cells, decrease the number of foam cells, and/or decrease the rate at which foam cells are formed. In certain instances, a foam cell dies and subsequently ruptures. In certain instances, the rupture of a foam cell deposits oxidized cholesterol into the artery wall. In some embodiments, the methods and compositions described herein prevent the deposition of oxidized cholesterol deposited onto an artery wall, decrease the amount of oxidized cholesterol deposited onto an artery wall, and/or decrease the rate at which oxidized cholesterol is deposited onto an arterial wall. In certain instances, the arterial wall becomes inflamed due to the damage caused by the oxidized LDL. In some embodiments, the methods and compositions described herein prevent arterial wall inflammation, decrease the portion of an arterial wall that is inflamed, and/or decrease the severity of the inflammation. In certain instances, the inflammation of arterial walls results in (either partially or full) the expression of matrix metalloproteinase (MMP)-2, CD40 ligand, and tumor necrosis factor (TNF)-α. In some embodiments, the methods and compositions described herein prevent the expression of matrix metalloproteinase (MMP)-2, CD40 ligand, and tumor necrosis factor (TNF)-α, or decrease the amount of matrix metalloproteinase (MMP)-2, CD40 ligand, and tumor necrosis factor (TNF)-α, expressed. In certain instances, cells form a hard covering over the inflamed area. In some embodiments, the methods and compositions described herein prevent the formation of the hard covering, decrease the portion of an arterial wall affected by the hard covering, and/or decrease the rate at which the hard covering is formed. In certain instances, the cellular covering narrows an artery. In some embodiments, the methods and compositions described herein prevent arterial narrowing, decrease the portion of an artery that is narrowed, decrease the severity of the narrowing, and/or decrease the rate at which the artery is narrowed.

In certain instances, an atherosclerotic plaque results (partially or fully) in stenosis (i.e., the narrowing of blood vessel). In certain instances, stenosis results (partially or fully) in decreased blood flow. In some embodiments, a method and/or composition described herein treats stenosis and/or restinosis. In certain instances, the mechanical injury of stenotic atherosclerotic lesions by percutaneous intervention (e.g., balloon angioplasty or stenting) induces the development of neointimal hyperplasia. In certain instances, the acute injury of the vessel wall induces acute endothelial denudation and platelet adhesion, as well as apoptosis of SMCs in the medial vessel wall. In certain instances, the accumulation of phenotypically unique SMCs within the intimal layer in response to injury functions to restore the integrity of the arterial vessel wall but subsequently leads to the progressive narrowing of the vessel. In certain instances, monocyte recruitment triggers a more sustained and chronic inflammatory response. In some embodiments, methods and compositions disclosed herein inhibit the accumulation of phenotypically unique SMCs within the intimal layer. In some embodiments, methods and compositions disclosed herein inhibit the accumulation of phenotypically unique SMCs within the intimal layer in an individual treated by balloon angioplasty or stenting.

In certain instances, the rupture of an atherosclerotic plaque results (partially or fully) in an infarction (e.g., myocardial infarction or stroke) to a tissue. In certain instances, myocardial MIF expression is upregulated in surviving cardiomyocytes and macrophages following cute myocardial ischemic injury. In certain instances, hypoxia and oxidative stress induce the secretion of MIF from cardiomyocytes through an atypical protein kinase C-dependent export mechanism and result in extracellular signal-regulated kinase activation. In certain instances, increased serum concentrations of MIF are detected in individuals with acute myocardial infarction. In certain instances, MIF contributes to macrophage accumulation in infarcted regions and to the proinflammatory role of myocyte-induced damage during infarction. In some embodiments, a method and/or composition described herein treats an infarction. In certain instances, reperfusion injury follows an infarction. In some embodiments, a method and/or composition described herein treats reperfusion injury.

In some embodiments, an antibody disclosed herein is administered to identify and/or locate an atherosclerotic plaque. In some embodiments, the antibody is labeled for imaging. In some embodiments, the antibody is labeled for medical imaging. In some embodiments, the antibody is labeled for radio-imaging, PET imaging, MRI imaging, and fluorescent imaging. In some embodiments, the antibody localizes to areas of the circulatory system with high concentrations of MIF. In some embodiments, an area of the circulatory system with high concentrations of MIF is an atherosclerotic plaque. In some embodiments, the labeled antibodies are detected by any suitable method (e.g., by use of a gamma camera, MRI, PET scanner, x-ray computed tomography (CT), functional magnetic resonance imaging (fMRI), and single photon emission computed tomography (SPECT)).

### Abdominal Aortic Aneurysm

In certain instances, an atherosclerotic plaque results (partially or fully) in the development of an aneurysm. In some embodiments, the methods and compositions described herein are administered to treat an aneurysm. In some embodiments, the methods and compositions described herein are administered to treat an abdominal aortic aneurysm (“AAA”). As used herein, an “abdominal aortic aneurysm” is a localized dilatation of the abdominal aorta characterized by at least a 50% increase over normal arterial diameter. In some embodiments, the methods and compositions described herein decrease the dilation of the abdominal aorta.

In certain instances, abdominal aortic aneurysms result (partially or fully) from a breakdown of structural proteins (e.g., elastin and collagen). In some embodiments, a method and/or composition disclosed herein partially or fully inhibits the breakdown of a structural protein (e.g., elastin and collagen). In some embodiments, a method and/or composition disclosed herein facilitates the regeneration of a structural protein (e.g., elastin and collagen). In certain instances, the breakdown of structural proteins is caused by activated MMPs. In some embodiments, a method and/or composition disclosed herein partially or fully inhibits the activation of an MMP. In some embodiments, a composition and/or method disclosed herein inhibits the upregulation of MMP-1, MMP-9 or MMP-12. In certain instances, MMPs are activated following infiltration of a section of the abdominal aorta by leukocytes (e.g., macrophages and neutrophils).

In some embodiments, the methods and compositions described herein decrease the infiltration of leukocytes. In certain instances, the MIF is upregulated in early abdominal aortic aneurysm. In certain instances, leukocytes follow a MIF gradient to a section of the abdominal aorta that is susceptible to the development of an AAA (e.g., the section of the aorta affected by an atherosclerotic plaque, infection, cystic medial necrosis, arteritis, trauma, an anastomotic disruption producing pseudoaneurysms). In some embodiments, a method and/or composition disclosed herein partially or fully inhibits the activity of MIF. In some embodiments, a method and/or composition disclosed herein partially or fully inhibits the ability of MIF to function as a chemokine for macrophages and neutrophils.

In some embodiments, an antibody disclosed herein is administered to identify and/or locate an AAA in an individual in need thereof. In some embodiments, an individual in need thereof displays one or more risk factors for developing an AAA (e.g., 60 years of age or older; male; cigarette smoking; high blood pressure; high serum cholesterol; diabetes mellitus; atherosclerosis). In some embodiments, the antibody is labeled for imaging. In some embodiments, the antibody is labeled for medical imaging. In some embodiments, the antibody is labeled for radio-imaging, PET imaging, MRI imaging, and fluorescent imaging. In some embodiments, the antibody localizes to areas of the circulatory system with high concentrations of MIF. In some embodiments, an area of the circulatory system with high concentrations of MIF is an AAA. In some embodiments, the labeled antibodies are detected by any suitable method (e.g., by use of a gamma camera, MRI, PET scanner, x-ray computed tomography (CT), functional magnetic resonance imaging (fMRI), and single photon emission computed tomography (SPECT)).

### Miscellaneous Disorders

In some embodiments, a method and/or composition described herein treats a T-cell mediated autoimmune disorder. In certain instances, a T-cell mediated autoimmune disorder is characterized by a T-cell mediated immune response against self (e.g., native cells and tissues). Examples of T-cell mediated autoimmune disorders include, but are not limited to colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjögren's syndrome, systemic lupus erythematosus (SLE), vasculitis syndromes (e.g., giant cell arteritis, Behcet's disease & Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In some embodiments, a method and/or composition described herein treats pain. Pain includes, but is not limited to acute pain, acute inflammatory pain, chronic inflammatory pain and neuropathic pain.

In some embodiments, a method and/or composition described herein treats hypersensitivity. As used herein, “hypersensitivity” refers to an undesirable immune system response. Hypersensitivity is divided into four categories. Type I hypersensitivity includes allergies (e.g., Atopy, Anaphylaxis, or Asthma). Type II hypersensitivity is cytotoxic/antibody mediated (e.g., Autoimmune hemolytic anemia, Thrombocytopenia, Erythroblastosis fetalis, or Goodpasture's syndrome). Type III is immune complex diseases (e.g., Serum sickness, Arthus reaction, or SLE). Type IV is delayed-type hypersensitivity (DTH), Cell-mediated immune memory response, and antibody-independent (e.g., Contact dermatitis, Tuberculin skin test, or Chronic transplant rejection).

As used herein, “allergy” means a disorder characterized by excessive activation of mast cells and basophils by IgE. In certain instances, the excessive activation of mast cells and basophils by IgE results (either partially or fully) in an inflammatory response. In certain instances, the inflammatory response is local. In certain instances, the inflammatory response results in the narrowing of airways (i.e., bronchoconstriction). In certain instances, the inflammatory response results in inflammation of the nose (i.e., rhinitis). In certain instances, the inflammatory response is systemic (i.e., anaphylaxis).

In some embodiments, a method and/or composition described herein treats angiogenesis. As used herein, “angiogenesis” refers to the formations of new blood vessels. In certain instances, angiogenesis occurs with chronic inflammation. In certain instances, angiogenesis is induced by monocytes and/or macrophages. In some embodiments, a method and/or composition disclosed herein inhibits angiogenesis. In certain instances, MIF is expressed in endothelial progenitor cells. In certain instances, MIF is expressed in tumor-associated neovasculature.

In some embodiments the present invention comprises a method of treating a neoplasia. In certain instances, a neoplastic cell induces an inflammatory response. In certain instances, part of the inflammatory response to a neoplastic cell is angiogenesis. In certain instances, angiogenesis facilitates the development of a neoplasia. In some embodiments, the neoplasia is: angiosarcoma, Ewing sarcoma, osteosarcoma, and other sarcomas, breast carcinoma, cecum carcinoma, colon carcinoma, lung carcinoma, ovarian carcinoma, pharyngeal carcinoma, rectosigmoid carcinoma, pancreatic carcinoma, renal carcinoma, endometrial carcinoma, gastric carcinoma, liver carcinoma, head and neck carcinoma, breast carcinoma and other carcinomas, Hodgkins lymphoma and other lymphomas, malignant and other melanomas, parotid tumor, chronic lymphocytic leukemia and other leukemias, astrocytomas, gliomas, hemangiomas, retinoblastoma, neuroblastoma, acoustic neuroma, neurofibroma, trachoma and pyogenic granulomas.

Disclosed herein, in some embodiments, are methods of promoting neovascularization comprising administering to said individual MIF or a MIF analogue.

As used herein, “sepsis” is a disorder characterized by whole-body inflammation. In certain instances, inhibiting the expression or activity of MIF increases the survival rate of individuals with sepsis. In some embodiments, a method and/or composition described herein treats sepsis. In certain instances, sepsis results in (either partially or fully) myocardial dysfunction (e.g., myocardial dysfunction). In some embodiments, a method and/or composition described herein treats myocardial dysfunction (e.g., myocardial dysfunction) resulting from sepsis.

In certain instances, MIF induces kinase activation and phosphorylation in the heart (i.e., indicators of cardiac depression). In some embodiments, a method and/or composition described herein treats myocardial dysfunction (e.g., myocardial dysfunction) resulting from sepsis.

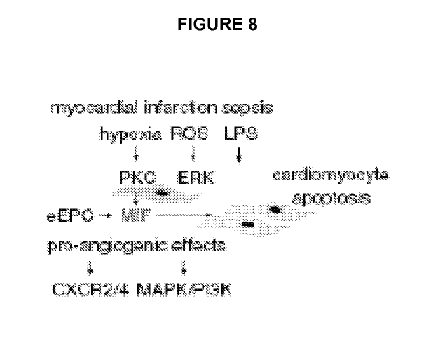

In certain instances, LPS induces the expression of MIF. In certain instances, MIF is induced by endotoxins during sepsis and functions as an initiating factor in myocardial inflammatory responses, cardiac myocyte apoptosis, and cardiac dysfunction (FIG. 8).

In some embodiments, the methods and compositions described herein inhibit myocardial inflammatory responses resulting from endotoxin exposure. In some embodiments, the methods and compositions described herein inhibit cardiac myocyte apoptosis resulting from endotoxin exposure. In some embodiments, the methods and compositions described herein inhibit cardiac dysfunction resulting from endotoxin exposure.

In certain instances, inhibition of MIF results in (either partially or fully) a significant increase in survival factors (e.g., Bcl-2, Bax, and phospho-Akt) and an improvement in cardiomyocyte survival and myocardial function. In some embodiments, the methods and compositions described herein increase the expression of Bcl-2, Bax or phospho-Akt.

In certain instances, MIF mediates the late and prolonged cardiac depression after burn injury associated and/or major tissue damage. In some embodiments, a method and/or composition described herein treats prolonged cardiac depression after burn injury. In some embodiments, a method and/or composition described herein treats prolonged cardiac depression after major tissue damage.

In certain instances, MIF is released from the lungs during sepsis.

In certain instances, antibody neutralization of MIF inhibits the onset of and reduced the severity of autoimmune myocarditis. In some embodiments, a method and/or composition described herein treats autoimmune myocarditis.

### Combinations

Disclosed herein, in certain embodiments, are methods and pharmaceutical compositions for modulating a disorder of a cardiovascular system, comprising a synergistic combination of (a) an antibody that inhibits (i) MIF binding to CXCR2 and CXCR4 and/or (ii) MIF-activation of CXCR2 and CXCR4; (iii) the ability of MIF to form a homomultimer; or a combination thereof; and (b) a second active agent.

Disclosed herein, in certain embodiments, are methods and pharmaceutical compositions for modulating a disorder of a cardiovascular system, comprising a synergistic combination of (a) an antibody that inhibits (i) MIF binding to CXCR2 and CXCR4 and/or (ii) MIF-activation of CXCR2 and CXCR4; (iii) the ability of MIF to form a homomultimer; or a combination thereof; and (b) a second active agent selected from an agent that treats a disorder a component of which is inflammation.

Disclosed herein, in certain embodiments, are methods and pharmaceutical compositions for modulating a disorder of a cardiovascular system, comprising a synergistic combination of (a) an antibody that inhibits (i) MIF binding to CXCR2 and CXCR4 and/or (ii) MIF-activation of CXCR2 and CXCR4; (iii) the ability of MIF to form a homomultimer; or a combination thereof; and (b) a second active agent selected from an agent a side-effect of which is undesired inflammation. In certain instances, statins (e.g., atorvastatin, lovastatin and simvastatin) induce inflammation. In certain instances, administration of a statin results (partially or fully) in myositis.

As used herein, the terms “pharmaceutical combination,” “administering an additional therapy,” “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to an individual simultaneously in the form of a single entity or dosage. The term “non-fixed combination” means that at least one of the agents described herein, and at least one co-agent, are administered to an individual as separate entities either simultaneously, concurrently or sequentially with variable intervening time limits, wherein such administration provides effective levels of the two or more agents in the body of the individual. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration,” “administered in combination with” and their grammatical equivalents are meant to encompass administration of the selected therapeutic agents to a single individual, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

In some embodiments, the co-administration of (a) an antibody disclosed herein; and (b) a second active agent allows (partially or fully) a medical professional to increase the prescribed dosage of the inflammatory disorder agent. In certain instances, statin-induced myositis is dose-dependent. In some embodiments, prescribing the active agent allows (partially or fully) a medical professional to increase the prescribed dosage of statin.

In some embodiments, the co-administration of (a) an antibody; and (b) a second active agent enables (partially or fully) a medical professional to prescribe the second active agent (i.e., co-administration rescues the inflammatory disorder agent).

In some embodiments, the second active agent is an active agent that targets HDL levels by indirect means (e.g. CETP inhibition). In some embodiments, combining a non-selective HDL therapy with an antibody disclosed herein; (2) a modulator of an interaction between RANTES and Platelet Factor 4; or (3) combinations thereof converts the second active agent that targets HDL levels by indirect means into a more efficacious therapy.

In some embodiments, the second active agent is administered before, after, or simultaneously with the modulator of inflammation.

### Pharmaceutical Therapies

In some embodiments, the second active agent is niacin, a fibrate, a statin, a Apo-A1 mimetic peptide (e.g., DF-4, Novartis), an apoA-I transcriptional up-regulator, an ACAT inhibitor, a CETP modulator, Glycoprotein (GP) IIb/IIIa receptor antagonists, P2Y12 receptor antagonists, Lp-PLA2-inhibitors, an anti-TNF agent, an IL-1 receptor antagonist, an IL-2 receptor antagonist, a cytotoxic agent, an immunomodulatory agent, an antibiotic, a T-cell co-stimulatory blocker, a disorder-modifying anti-rheumatic agent, a B cell depleting agent, an immunosuppressive agent, an anti-lymphocyte antibody, an alkylating agent, an anti-metabolite, a plant alkaloid, a terpenoids, a topoisomerase inhibitor, an anti-tumor antibiotic, a monoclonal antibody, a hormonal therapy (e.g., aromatase inhibitors), or combinations thereof.

In some embodiments, the second active is niacin, bezafibrate; ciprofibrate; clofibrate; gemfibrozil; fenofibrate; DF4 (Ac-D-W-F-K-A-F-Y-D-K-V-A-E-K-F-K-E-A-F-NH2); DF5; RVX-208 (Resverlogix); avasimibe; pactimibe sulfate (CS-505); CI-1011 (2,6-diisopropylphenyl[(2,4,6-triisopropylphenyl)acetyl]sulfamate); CI-976 (2,2-dimethyl-N-(2,4,6-trimethoxyphenyl)dodecanamide); VULM1457 (1-(2,6-diisopropyl-phenyl)-3-[4-(4′-nitrophenylthio)phenyl]urea); CI-976 (2,2-dimethyl-N-(2,4,6-trimethoxyphenyl)dodecanamide); E-5324 (n-butyl-N′-(2-(3-(5-ethyl-4-phenyl-1H-imidazol-1-yl)propoxy)-6-methylphenyl)urea); HL-004 (N-(2,6-diisopropylphenyl)tetradecylthioacetamide); KY-455 (N-(4,6-dimethyl-1-pentylindolin-7-yl)-2,2-dimethylpropanamide); FY-087 (N-[2-[N′-pentyl-(6,6-dimethyl-2,4-heptadiynyl)amino]ethyl]-(2-methyl-1-naphthyl-thio)acetamide); MCC-147 (Mitsubishi Pharma); F 12511 ((S)-2′,3′,5′-trimethyl-4′-hydroxy-alpha-dodecylthioacetanilide); SMP-500 (Sumitomo Pharmaceuticals); CL 277082 (2,4-difluoro-phenyl-N[[4-(2,2-dimethylpropyl)phenyl]methyl]-N-(hepthyl)urea); F-1394 ((1s,2s)-2-[3-(2,2-dimethylpropyl)-3-nonylureido]aminocyclohexane-1-yl 3-[N-(2,2,5,5-tetramethyl-1,3-dioxane-4-carbonyl)amino]propionate); CP-113818 (N-(2,4-bis(methylthio)-6-methylpyridin-3-yl)-2-(hexylthio)decanoic acid amide); YM-750; torcetrapib; anacetrapid; JTT-705 (Japan Tobacco/Roche); abciximab; eptifibatide; tirofiban; roxifiban; variabilin; XV 459 (N(3)-(2-(3-(4-formamidinophenyl)isoxazolin-5-yl)acetyl)-N(2)-(1-butyloxycarbonyl)-2,3-diaminopropionate); SR 121566A (3-[N-{4-[4-(aminoiminomethyl)phenyl]-1,3-thiazol-2-yl}-N-(1-carboxymethylpiperid-4-yl)amino]propionic acid, trihydrochloride); FK419 ((S)-2-acetylamino-3-[(R)[1-[3-(piperidin-4-yl)propionyl]piperidin-3-ylcarbonyl]amino]propionic acid trihydrate); clopidogrel; prasugrel; cangrelor; AZD6140 (AstraZeneca); MRS 2395 (2,2-Dimethyl-propionic acid 3-(2-chloro-6-methylaminopurin-9-yl)-2-(2,2-dimethyl-propionyloxymethyl)-propyl ester); BX 667 (Berlex Biosciences); BX 048 (Berlex Biosciences); darapladib (SB 480848); SB-435-495 (GlaxoSmithKline); SB-222657 (GlaxoSmithKline); SB-253514 (GlaxoSmithKline); alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, sulfasalazine, etanercept, adalimumab, infliximab, abatacept, rituximab, trastuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-1b, interferon beta-1a, tizanidine, baclofen, mesalazine, asacol, pentasa, mesalamine, balsalazide, olsalazine, 6-mercaptopurine, A1N457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-1beta monoclonal antibody, Novartis), CNTO 1275 (Fully Human Anti-IL-12 Monoclonal Antibody, Centocor), (3S)-N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA (Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; cisplatin; carboplatin; oxaliplatin; mechlorethamine; cyclophosphamide; chlorambucil; vincristine; vinblastine; vinorelbine; vindesine; azathioprine; mercaptopurine; fludarabine; pentostatin; cladribine; 5-fluorouracil (5FU); floxuridine (FUDR); cytosine arabinoside; methotrexate; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; trastuzumab; cetuximab; rituximab; bevacizumab; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; milatuzumab; milatuzumab conjugated to doxorubicin; or combinations thereof.

### Gene Therapy

Disclosed herein, in certain embodiments, is a composition for modulating an inflammatory disorder, comprising a combination of (a) an antibody disclosed herein; and (b) gene therapy. Disclosed herein, in certain embodiments, is a method for modulating an inflammatory disorder, comprising co-administering a combination of (a) an antibody disclosed herein; and (b) gene therapy.

In some embodiments, the gene therapy comprises modulating the concentration of a lipid and/or lipoprotein (e.g., HDL) in the blood of an individual in need thereof. In some embodiments, modulating the concentration of a lipid and/or lipoprotein (e.g., HDL) in the blood comprises transfecting DNA into an individual in need thereof. In some embodiments, the DNA encodes an Apo A1 gene, an LCAT gene, an LDL gene, an IL-4 gene, an IL-10 gene, an IL-1ra gene, a galectin-3 gene, or combinations thereof. In some embodiments, the DNA is transfected into a liver cell.

In some embodiments, the DNA is transfected into a liver cell via use of ultrasound. For disclosures of techniques related to transfecting ApoA1 DNA via use of ultrasound see U.S. Pat. No. 7,211,248, which is hereby incorporated by reference for those disclosures.

In some embodiments, an individual is administered a vector engineered to carry the human gene (the “gene vector”). For disclosures of techniques for creating an LDL gene vector see U.S. Pat. No. 6,784,162, which is hereby incorporated by reference for those disclosures. In some embodiments, the gene vector is a retrovirus. In some embodiments, the gene vector is not a retrovirus (e.g. it is an adenovirus; a lentivirus; or a polymeric delivery system such as METAFECTENE, SUPERFECT®, EFFECTENE®, or MIRUS TRANSIT). In certain instances, a retrovirus, adenovirus, or lentivirus will have a mutation such that the virus is rendered incompetent.

In some embodiments, the vector is administered in vivo (i.e., the vector is injected directly into the individual, for example into a liver cell), ex vivo (i.e., cells from the individual are grown in vitro and transduced with the gene vector, embedded in a carrier, and then implanted in the individual), or a combination thereof.

In certain instances, after administration of the gene vector, the gene vector infects the cells at the site of administration (e.g. the liver). In certain instances the gene sequence is incorporated into the individual's genome (e.g. when the gene vector is a retrovirus). In certain instances the therapy will need to be periodically re-administered (e.g. when the gene vector is not a retrovirus). In some embodiments, the therapy is re-administered annually. In some embodiments, the therapy is re-administered semi-annually. In some embodiments, the therapy is re-administered when the individual's HDL level decreases below about 60 mg/dL. In some embodiments, the therapy is re-administered when the individual's HDL level decreases below about 50 mg/dL. In some embodiments, the therapy is re-administered when the individual's HDL level decreases below about 45 mg/dL. In some embodiments, the therapy is re-administered when the individual's HDL level decreases below about 40 mg/dL. In some embodiments, the therapy is re-administered when the individual's HDL level decreases below about 35 mg/dL. In some embodiments, the therapy is re-administered when the individual's HDL level decreases below about 30 mg/dL.

### RNAi Therapies

Disclosed herein, in certain embodiments, is composition for modulating an inflammatory disorder, comprising a combination of (a) an antibody disclosed herein; and (b) an RNAi molecule designed to silence the expression of a gene that participates in the development and/or progression of a MIF-mediated disorder (the “target gene”). Disclosed herein, in certain embodiments, is a method for modulating an inflammatory disorder, comprising administering a combination of (a) an antibody disclosed herein; and (b)) an RNAi molecule designed to silence the expression of a gene that participates in the development and/or progression of a MIF-mediated disorder (the “target gene”). In some embodiments, the target gene is Apolipoprotein B (Apo B), Heat Shock Protein 110 (Hsp 110), Proprotein Convertase Subtilisin Kexin 9 (Pcsk9), CyD1, TNF-α, IL-1β, Atrial Natriuretic Peptide Receptor A (NPRA), GATA-3, Syk, VEGF, MIP-2, FasL, DDR-1, C5aR, AP-1, or combinations thereof.

In some embodiments, the target gene is silenced by RNA interference (RNAi). In some embodiments, the RNAi therapy comprises use of an siRNA molecule. In some embodiments, a double stranded RNA (dsRNA) molecule with sequences complementary to an mRNA sequence of a gene to be silenced (e.g., Apo B, Hsp 110 and Pcsk9) is generated (e.g by PCR). In some embodiments, a 20-25 bp siRNA molecule with sequences complementary to an mRNA sequence of a gene to be silenced is generated. In some embodiments, the 20-25 bp siRNA molecule has 2-5 bp overhangs on the 3′ end of each strand, and a 5′ phosphate terminus and a 3′ hydroxyl terminus. In some embodiments, the 20-25 bp siRNA molecule has blunt ends. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, an siRNA molecule is “fully complementary” (i.e., 100% complementary) to the target gene. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target gene. In some embodiments, there is a 1 bp mismatch, a 2 bp mismatch, a 3 bp mismatch, a 4 bp mismatch, or a 5 bp mismatch.

In certain instances, after administration of the dsRNA or siRNA molecule, cells at the site of administration (e.g. the cells of the liver and/or small intestine) are transformed with the dsRNA or siRNA molecule. In certain instances following transformation, the dsRNA molecule is cleaved into multiple fragments of about 20-25 bp to yield siRNA molecules. In certain instances, the fragments have about 2 bp overhangs on the 3′ end of each strand.

In certain instances, an siRNA molecule is divided into two strands (the guide strand and the anti-guide strand) by an RNA-induced Silencing Complex (RISC). In certain instances, the guide strand is incorporated into the catalytic component of the RISC (i.e. argonaute). In certain instances, the guide strand specifically binds to a complementary RB1 mRNA sequence. In certain instances, the RISC cleaves an mRNA sequence of a gene to be silenced. In certain instances, the expression of the gene to be silenced is down-regulated.

In some embodiments, a sequence complementary to an mRNA sequence of a target gene is incorporated into a vector. In some embodiments, the sequence is placed between two promoters. In some embodiments, the promoters are orientated in opposite directions. In some embodiments, the vector is contacted with a cell. In certain instances, a cell is transformed with the vector. In certain instances following transformation, sense and anti-sense strands of the sequence are generated. In certain instances, the sense and anti-sense strands hybridize to form a dsRNA molecule which is cleaved into siRNA molecules. In certain instances, the strands hybridize to form an siRNA molecule. In some embodiments, the vector is a plasmid (e.g pSUPER; pSUPER.neo; pSUPER.neo+gfp).

In some embodiments, an siRNA molecule is administered to in vivo (i.e., the vector is injected directly into the individual, for example into a liver cell or a cell of the small intestine, or into the blood stream).

In some embodiments, a siRNA molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically-acceptable excipients. For methods of formulating and administering a nucleic acid molecule to an individual in need thereof see Akhtar et al., 1992, Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Symp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, an siRNA molecule described herein is administered to the liver by any suitable manner (see e.g., Wen et al., 2004, World J Gastroenterol., 10, 244-9; Murao et al., 2002, Pharm Res., 19, 1808-14; Liu et al., 2003, Gene Ther., 10, 180-7; Hong et al., 2003, J Pharm Pharmacol., 54, 51-8; Herrmann et al., 2004, Arch Virol., 149, 1611-7; and Matsuno et al., 2003, Gene Ther., 10, 1559-66).

In some embodiments, an siRNA molecule described herein is administered iontophoretically, for example to a particular organ or compartment (e.g., the liver or small intestine). Non-limiting examples of iontophoretic delivery are described in, for example, WO 03/043689 and WO 03/030989, which are hereby incorporated by reference for such disclosures.

In some embodiments, an siRNA molecule described herein is administered systemically (i.e., in vivo systemic absorption or accumulation of an siRNA molecule in the blood stream followed by distribution throughout the entire body). Administration routes contemplated for systemic administration include, but are not limited to, intravenous, subcutaneous, portal vein, intraperitoneal, and intramuscular. Each of these administration routes exposes the siRNA molecules of the invention to an accessible diseased tissue (e.g., liver).

In certain instances the therapy will need to be periodically re-administered. In some embodiments, the therapy is re-administered annually. In some embodiments, the therapy is re-administered semi-annually. In some embodiments, the therapy is administered monthly. In some embodiments, the therapy is administered weekly. In some embodiments, the therapy is re-administered when the individual's HDL level decreases below about 60 mg/dL. In some embodiments, the therapy is re-administered when the individual's HDL level decreases below about 50 mg/dL. In some embodiments, the therapy is re-administered when the individual's HDL level decreases below about 45 mg/dL. In some embodiments, the therapy is re-administered when the individual's HDL level decreases below about 40 mg/dL. In some embodiments, the therapy is re-administered when the individual's HDL level decreases below about 35 mg/dL. In some embodiments, the therapy is re-administered when the individual's HDL level decreases below about 30 mg/dL.

For disclosures of techniques related to silencing the expression of Apo B and/or Hsp110 see U.S. Pub. No. 2007/0293451 which is hereby incorporated by reference for such disclosures. For disclosures of techniques related to silencing the expression of Pcsk9 see U.S. Pub. No. 2007/0173473 which is hereby incorporated by reference for such disclosures.

### Antisense Therapies

Disclosed herein, in certain embodiments, is a composition for modulating an inflammatory disorder, comprising a combination of (a) an antibody disclosed herein; and (b) an antisense molecule designed to inhibit the expression of and/or activity of a DNA or RNA sequence that participates in the development and/or progression of a MIF-mediated disorder (the “target sequence”). Disclosed herein, in certain embodiments, is a method for modulating an inflammatory disorder, comprising co-administering (a) an antibody disclosed herein; and (b) an antisense molecule designed to inhibit the expression of and/or activity of a DNA or RNA sequence that participates in the development and/or progression of a MIF-mediated disorder (the “target sequence”). In some embodiments, inhibiting the expression of and/or activity of a target sequence comprises use of an antisense molecule complementary to the target sequence. In some embodiments, the target sequence is microRNA-122 (miRNA-122 or mRNA-122), secretory phospholipase A2 (sPLA2), intracellular adhesion molecule-1 (ICAM-1), GATA-3, NF-κB, Syk, or combinations thereof. In certain instances, inhibiting the expression of and/or activity of miRNA-122 results (partially or fully) in a decrease in the concentration of cholesterol and/or lipids in blood.

In some embodiments, an antisense molecule that is complementary to a target sequence is generated (e.g. by PCR). In some embodiments, the antisense molecule is about 15 to about 30 nucleotides. In some embodiments, the antisense molecule is about 17 to about 28 nucleotides. In some embodiments, the antisense molecule is about 19 to about 26 nucleotides. In some embodiments, the antisense molecule is about 21 to about 24 nucleotides. For techniques for generating RNA sequences see Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); Current Protocols in Nucleic Acid Chemistry John Wiley & Sons, Inc., New York, 2000) which are hereby incorporated by reference for such disclosure.

In some embodiments, the antisense molecules are single-stranded, double-stranded, circular or hairpin. In some embodiments, the antisense molecules contain structural elements (e.g., internal or terminal bulges, or loops).

In some embodiments, an antisense molecule is “fully complementary” (i.e., 100% complementary) to the target sequence. In some embodiments, an antisense molecule is “mostly complementary” (e.g., 99%, 98%, 97%, 96%, 95%, 94%, 93%, 92%, 91%, 90%, 85%, 80%, 75%, or 70% complementary) to the target RNA sequence. In some embodiments, there is a 1 bp mismatch, a 2 bp mismatch, a 3 bp mismatch, a 4 bp mismatch, or a 5 bp mismatch.

In some embodiments, the antisense molecule hybridizes to the target sequence. As used herein, “hybridize” means the pairing of nucleotides of an antisense molecule with corresponding nucleotides of the target sequence. In certain instances, hybridization involves the formation of one or more hydrogen bonds (e.g., Watson-Crick, Hoogsteen or reversed Hoogsteen hydrogen bonding) between the pairing nucleotides.

In certain instances, hybridizing results (partially or fully) in the degradation, cleavage, and/or sequestration of the RNA sequence.

In some embodiments, a siRNA molecule is formulated with a delivery vehicle (e.g., a liposome, a biodegradable polymer, a cyclodextrin, a PLGA microsphere, a PLCA microsphere, a biodegradable nanocapsule, a bioadhesive microsphere, or a proteinaceous vector), carriers and diluents, and other pharmaceutically-acceptable excipients. For methods of formulating and administering a nucleic acid molecule to an individual in need thereof see Akhtar et al., 1992, Trends Cell Bio., 2, 139; Delivery Strategies for Antisense Oligonucleotide Therapeutics, ed. Akhtar, 1995; Maurer et al., 1999, Mol. Membr. Biol., 16, 129-140; Hofland and Huang, 1999, Handb. Exp. Pharmacol., 137, 165-192; Lee et al., 2000, ACS Symp. Ser., 752, 184-192; Beigelman et al., U.S. Pat. No. 6,395,713; Sullivan et al., PCT WO 94/02595; Gonzalez et al., 1999, Bioconjugate Chem., 10, 1068-1074; Wang et al., International PCT publication Nos. WO 03/47518 and WO 03/46185; U.S. Pat. No. 6,447,796; US Patent Application Publication No. US 2002130430; O'Hare and Normand, International PCT Publication No. WO 00/53722; and U.S. Patent Application Publication No. 20030077829; U.S. Provisional patent application No. 60/678,531, all of which are hereby incorporated by reference for such disclosures.

In some embodiments, an siRNA molecule described herein is administered to the liver by any suitable manner (see e.g., Wen et al., 2004, World J Gastroenterol., 10, 244-9; Murao et al., 2002, Pharm Res., 19, 1808-14; Liu et al., 2003, Gene Ther., 10, 180-7; Hong at al., 2003, J Pharm Pharmacol., 54, 51-8; Herrmann at al., 2004, Arch Virol., 149, 1611-7; and Matsuno at al., 2003, Gene Ther., 10, 1559-66).

In some embodiments, an siRNA molecule described herein is administered iontophoretically, for example to a particular organ or compartment (e.g., the liver or small intestine). Non-limiting examples of iontophoretic delivery are described in, for example, WO 03/043689 and WO 03/030989, which are hereby incorporated by reference for such disclosures.

In some embodiments, an siRNA molecule described herein is administered systemically (i.e., in vivo systemic absorption or accumulation of an siRNA molecule in the blood stream followed by distribution throughout the entire body). Administration routes contemplated for systemic administration include, but are not limited to, intravenous, subcutaneous, portal vein, intraperitoneal, and intramuscular. Each of these administration routes exposes the siRNA molecules of the invention to an accessible diseased tissue (e.g., liver).

In certain instances the therapy will need to be periodically re-administered. In some embodiments, the therapy is re-administered annually. In some embodiments, the therapy is re-administered semi-annually. In some embodiments, the therapy is administered monthly. In some embodiments, the therapy is administered weekly. In some embodiments, the therapy is re-administered when the individual's HDL level decreases below about 60 mg/dL. In some embodiments, the therapy is re-administered when the individual's HDL level decreases below about 50 mg/dL. In some embodiments, the therapy is re-administered when the individual's HDL level decreases below about 45 mg/dL. In some embodiments, the therapy is re-administered when the individual's HDL level decreases below about 40 mg/dL. In some embodiments, the therapy is re-administered when the individual's HDL level decreases below about 35 mg/dL. In some embodiments, the therapy is re-administered when the individual's HDL level decreases below about 30 mg/dL.

For disclosures of techniques related to silencing the expression of miRNA-122 see WO 07/027,775A2 which is hereby incorporated by reference for such disclosures.

### Device-Mediated Therapies

In some embodiments, the device mediated strategy comprises removing a lipid from an HDL molecule in an individual in need thereof (delipification), removing an LDL molecule from the blood or plasma of an individual in need thereof (delipification), or a combination thereof. For disclosures of techniques for removing a lipid from an HDL molecule and removing an LDL molecule from the blood or plasma of an individual in need thereof see U.S. Pub. No. 2008/0230465, which is hereby incorporated by reference for those disclosures.

In certain instances, the delipification therapy will need to be periodically re-administered. In some embodiments, the delipification therapy is re-administered annually. In some embodiments, the delipification therapy is re-administered semi-annually. In some embodiments, the delipification therapy is re-administered monthly. In some embodiments, the delipification therapy is re-administered semi-weekly. In some embodiments, the therapy is re-administered when the individual's HDL level decreases below about 60 mg/dL. In some embodiments, the therapy is re-administered when the individual's HDL level decreases below about 50 mg/dL. In some embodiments, the therapy is re-administered when the individual's HDL level decreases below about 45 mg/dL. In some embodiments, the therapy is re-administered when the individual's HDL level decreases below about 40 mg/dL. In some embodiments, the therapy is re-administered when the individual's HDL level decreases below about 35 mg/dL. In some embodiments, the therapy is re-administered when the individual's HDL level decreases below about 30 mg/dL.

### Pharmaceutical Compositions

Disclosed herein, in certain embodiments, is a pharmaceutical composition for modulating an inflammation and/or a MIF-mediated disorder comprising a therapeutically-effective amount of an antibody disclosed herein.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York N.Y., 1980; and Pharmaceutical Dosage Forms and Drug Delivery Systems, Seventh Ed. (Lippincott Williams & Wilkins, 1999).

In certain embodiments, the pharmaceutical composition for modulating a disorder of a cardiovascular system further comprises a pharmaceutically acceptable diluent(s), excipient(s), or carrier(s). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

The pharmaceutical formulations described herein are optionally administered to an individual by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, modified release formulations, delayed release formulations, extended release formulations, pulsatile release formulations, multiparticulate formulations, and mixed immediate release and controlled release formulations.

In some embodiments, the pharmaceutical compositions described herein are formulated as multiparticulate formulations. In some embodiments, the pharmaceutical compositions described herein comprise a first population of particles and a second population of particles. In some embodiments, the first population comprises an active agent. In some embodiments, the second population comprises an active agent. In some embodiments, the dose of active agent in the first population is equal to the dose of active agent in the second population. In some embodiments, the dose of active agent in the first population is not equal to (e.g., greater than or less than) the dose of active agent in the second population.

In some embodiments, the active agent of the first population is released before the active agent of the second population. In some embodiments, the second population of particles comprises a modified-release (e.g., delayed-release, controlled-release, or extended release) coating. In some embodiments, the second population of particles comprises a modified-release (e.g., delayed-release, controlled-release, or extended release) matrix.

Coating materials for use with the pharmaceutical compositions described herein include, but are not limited to, polymer coating materials (e.g., cellulose acetate phthalate, cellulose acetate trimaletate, hydroxy propyl methylcellulose phthalate, polyvinyl acetate phthalate); ammonio methacrylate copolymers (e.g., Eudragit® RS and RL); poly acrylic acid and poly acrylate and methacrylate copolymers (e.g., Eudragite S and L, polyvinyl acetaldiethylamino acetate, hydroxypropyl methylcellulose acetate succinate, shellac); hydrogels and gel-forming materials (e.g., carboxyvinyl polymers, sodium alginate, sodium carmellose, calcium carmellose, sodium carboxymethyl starch, poly vinyl alcohol, hydroxyethyl cellulose, methyl cellulose, gelatin, starch, hydroxypropyl cellulose, hydroxypropyl methylcellulose, polyvinylpyrrolidone, crosslinked starch, microcrystalline cellulose, chitin, aminoacryl-methacrylate copolymer, pullulan, collagen, casein, agar, gum arabic, sodium carboxymethyl cellulose, (swellable hydrophilic polymers) poly(hydroxyalkyl methacrylate) (m. wt. ^{˜}5 k-5,000 k), polyvinylpyrrolidone (m. wt. ^{˜}10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ^{˜}30 k-300 k), polysaccharides such as agar, acacia, karaya, tragacanth, algins and guar, polyacrylamides, Polyox® polyethylene oxides (m. wt. ^{˜}100 k-5,000 k), AquaKeep® acrylate polymers, diesters of polyglucan, crosslinked polyvinyl alcohol and poly N-vinyl-2-pyrrolidone, sodium starch; hydrophilic polymers (e.g., polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid, other acrylic acid derivatives, sorbitan esters, natural gums, lecithins, pectin, alginates, ammonia alginate, sodium, calcium, potassium alginates, propylene glycol alginate, agar, arabic gum, karaya gum, locust bean gum, tragacanth gum, carrageens gum, guar gum, xanthan gum, scleroglucan gum); or combinations thereof. In some embodiments, the coating comprises a plasticiser, a lubricant, a solvent, or combinations thereof. Suitable plasticisers include, but are not limited to, acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

In some embodiments, the second population of particles comprises a modified release matrix material. Materials for use with the pharmaceutical compositions described herein include, but are not limited to microcrystalline cellulose, sodium carboxymethylcellulose, hydroxyalkylcelluloses (e.g., hydroxypropylmethylcellulose and hydroxypropylcellulose), polyethylene oxide, alkykelluloses (e.g., methylcellulose and ethylcellulose), polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate, or combinations thereof.

In some embodiments, the first population of particles comprises a cardiovascular disorder agent. In some embodiments, the second population of particles comprises a (1) a modulator of MIF; (2) a modulator of an interaction between RANTES and Platelet Factor 4; or (3) combinations thereof. In some embodiments, the first population of particles comprises a (1) a modulator of MIF; (2) a modulator of an interaction between RANTES and Platelet Factor 4; or (3) combinations thereof. In some embodiments, the second population of particles comprises a cardiovascular disorder agent.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active agent doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations disclosed herein are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a MIF receptor inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a MIF receptor inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the pharmaceutical compositions disclosed herein are optionally in a form of an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix and a suitable powder base such as lactose or starch.

Buccal formulations include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of a pharmaceutical compositions disclosed here are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) an active agent; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery. The rate of absorption is optionally slowed by using rate-controlling membranes or by trapping an active agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing an active agent optionally with carriers, optionally a rate controlling barrier to deliver a an active agent to the skin of the host at a controlled and predetermined rate over a prolonged period of time, and means to secure the device to the skin.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, an active agent disclosed herein is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

An active agent disclosed herein is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

An active agent disclosed herein is optionally used in the preparation of medicaments for the prophylactic and/or therapeutic treatment of inflammatory conditions or conditions that would benefit, at least in part, from amelioration. In addition, a method for treating any of the diseases or conditions described herein in an individual in need of such treatment, involves administration of pharmaceutical compositions containing an active agent disclosed herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

In the case wherein the individual's condition does not improve, upon the doctor's discretion the administration of an active agent disclosed herein is optionally administered chronically, that is, for an extended period of time, including throughout the duration of the individual's life in order to ameliorate or otherwise control or limit the symptoms of the individual's disease or condition.

In the case wherein the individual's status does improve, upon the doctor's discretion the administration of an active agent disclosed herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-400%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the individual's conditions has occurred, a maintenance dose is administered if necessary. Subsequently, the dosage or the frequency of administration, or both, is reduced, as a function of the symptoms, to a level at which the improved disease, disorder or condition is retained. In some embodiments, individuals require intermittent treatment on a long-term basis upon any recurrence of symptoms.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for an active agent disclosed herein are from about 0.01 to 3 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the MIF receptor inhibitor used, the disease or condition to be treated, the mode of administration, the requirements of the individual, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. An active agent disclosed herein exhibiting high therapeutic indices is preferred. The data obtained from cell culture assays and animal studies are optionally used in formulating a range of dosage for use in human. The dosage of such an active agent disclosed herein lies preferably within a range of circulating concentrations that include the ED50 with minimal toxicity. The dosage optionally varies within this range depending upon the dosage form employed and the route of administration utilized.

### EXAMPLES

The following specific examples are to be construed as illustrative, and not limiting of the disclosure or the claims.

### Example 1

### Cell Lines and Reagents

Human aortic (Schober, A., et al. (2004) Circulation 109, 380-385) and umbilical vein (Weber, K. S., et al. (1999) Eur. J. Immunol. 29, 700-712) endothelial cells (PromoCell), MonoMac6 cells (Weber, C., et al. (1993) Eur. J. Immunol. 23, 852-859) and Chinese hamster ovary (CHO) ICAM-1-transfectants (Ostermann, G., et al. (2002) Nat. Immunol. 3, 151-158) were used as described. Jurkat cells and RAW264.7 macrophages were transfected with pcDNA3-CXCR2. HL-60 cells were transfected with pcDNA3.1/V5-HisTOPO-TA-CD74 or vector control (Nucleofector Kit V, Amaxa). L1.2 cells were transfected with pcDNA3-CXCRs or pcDNA-CCR5 (UMR cDNA Resource Center) for assays on simian virus-40-transformed mouse microvascular endothelial cells (SVECs). Peripheral blood mononuclear cells were prepared from buffy coats, monocytes by adherence or immunomagnetic separation (Miltenyi), primary T cells by phytohaemaglutinin/interleukin-2 (Biosource) stimulation and/or immunomagnetic selection (antibody to CD3/M-450 Dynabeads), and neutrophils by Ficoll gradient centrifugation. Human embryonal kidney-CXCR2 transfectants (HEK293-CXCR2) have been described previously (Ben-Baruch, A., et al. (1997) Cytokine 9, 37-45).

Recombinant MIF was expressed and purified as described (Bernhagen, J., at al. (1993) Nature 365, 756-759). Chemokines were from PeproTech. Human VCAM-1.Fc chimera, blocking antibodies to CXCR1 (42705, 5A12), CXCR2 (48311), CXCR4 (44708, FABSP2 cocktail, R&D), human MIF and mouse MIF (NIHIII.D.9) (Lan, H. Y., et. al. (1997) J. Exp. Med. 185, 1455-1465), CD74 (M-B741, Pharmingen), β₂ integrin (TS1/18), α₄ integrin (HP2/1) (Weber, C., et al. (1996) J. Cell Biol. 134, 1063-1073) and CXCR2 (RII115), and antibody to α₄ integrin (327C) (Shamri, R., at al. (2005) Nat. Immunol. 6, 497-506) were used. PTX and B-oligomer were from Merck.

### Methods Used in Examples

### Adhesion Assays.

Arrest of calcein-AM (Molecular Probes)-labeled monocytes, T cells and L1.2 transfectants was quantified in parallel-wall chambers in flow (1.5 dynes/cm², 5 min) (Schober, A., at al. (2004) Circulation 109, 380-385; Ostermarm, G., at al. (2002) Nat. Immunol. 3, 151-158; Weber, C., at al. (1996) J. Cell Biol. 134, 1063-1073). Confluent endothelial cells, CHO-ICAM-1 cells, VCAM-1.Fc-coated plates and leukocytes were pretreated with MIF, chemokines or antibodies. CHO-ICAM-1 cells incubated with MIF (2 h) were stained with antibody to MIF Ka565 (Leng, L., at al. (2003) J. Exp. Med. 197, 1467-1476) and FITC-conjugated antibody.

### Chemotaxis Assays.

Using Transwell chambers (Costar), we quantified primary leukocyte migration toward MIF or chemokines by fluorescence microscopy or using calcein-AM labeling and FluoroBlok filters (Falcon). Cells were pretreated with PTX/B-oligomer, Ly294002, MIF (for desensitization), antibodies to CXCRs or CD74, or isotype IgG. Pore sizes and intervals were 5 μm and 3 h (monocytes), 3 μm and 1.5 h (T cells), and 3 mm and 1 h (neutrophils).

### Q-PCR and ELISA.

RNA was reverse-transcribed using oligo-dT primers. RTPCR was performed using QuantiTect Kit with SYBRGreen (Qiagen), specific primers and an MJ Opticon2 (Biozym). CXCL8 was quantified by Quantikine ELISA (R&D).

### α_{L}β₂ Integrin Activation Assay.

Monocytes stimulated with MIF or Mg²⁺/EGTA (positive control) were fixed, reacted with the antibody 327C and an FITC-conjugated antibody to mouse IgG. LFA-1 activation analyzed by flow cytometry is reported as the increase in mean fluorescent intensity (MFI) or relative to the positive control (Shamri, R., et al. (2005) Nat. Immunol. 6, 497-506).

### Calcium Mobilization.

Neutrophils or L1.2 CXCR2 transfectants were labeled with Fluo-4 AM (Molecular Probes). After the addition of the first or a subsequent stimulus (MIF, CXCL8 or CXCL7), MFI was monitored as a measure of cytosolic Ca²⁺ concentrations for 120 s using a BD FACSAria. L1.2 controls showed negligible calcium influx.

### Receptor-Binding Assays.

Because iodinated MIF is inactive (Leng, L., at al. (2003) J. Exp. Med. 197, 1467-1476; Kleemann, R., at al. (2002) J. Interferon Cytokine Res. 22, 351-363), competitive receptor binding (Hayashi, S., at al. (1995) J. Immunol. 154, 814-824) were performed using radioiodinated tracers (Amersham): [I¹²⁵]CXCL8, reconstituted at 4 nM (80 μCi/ml) to a final concentration of 40 pM; [I¹²⁵]CXCL12, reconstituted at 5 nM (100 μCi/ml) to a final concentration of 50 pM. For competition of [I¹²⁵]CXCL8 with MIF for CXCR2 binding or competition of [I¹²⁵]CXCL12 with MIF for CXCR4 binding in equilibrium binding assays, cold MIF and/or CXCL with tracers to HEK293-CXCR2 or CXCR4-bearing Jurkat cells were added. The analysis was performed by liquid scintillation counting. To calculate EC⁵⁰ and K_{d} values, a one-site receptor-ligand binding model was assumed and the Cheng/Prusoff-equation and GraphPad Prism were used.

For pull-down of biotin-MIF-CXCR complexes, HEK293-CXCR2 transfectants or controls were incubated with biotin-labeled MIF (Kleemann, R., at al. (2002) J. Interferon Cytokine Res. 22, 351-363), washed and lysed with coimmunoprecipitation (CoIP) buffer. Complexes were isolated from cleared lysates by streptavidin-coated magnetic beads (M280, Dynal) and analyzed by western blotting with antibody to CXCR2 or streptavidin-peroxidase. For flow cytometry, HEK293-CXCR2 transfectants or Jurkat cells pretreated with AMD3465 and/or a 20-fold excess of unlabeled MIF were incubated with fluorescein-labeled MIF and analyzed using a BD FACSCalibur.

### CXCR Internalization Assays.

HEK293-CXCR2 or Jurkat cells were treated with CXCL8 or CXCL12, respectively, treated with MIF, washed with acidic glycine-buffer, stained with antibodies to CXCR2 or CXCR4, and analyzed by flow cytometry. Internalization was calculated relative to surface expression of buffer-treated cells (100% control) and isotype control staining (0% control): geometric MFI[experimental]−MFI[0% control]/MFI[100% control]−MFI[0% control]×100.

### Co Localization of CXCR2 and CD74.

RAW264.7-CXCR2 transfectants were co stained with CXCR2 and rat antibody to mouse CD74 (In-1, Pharmingen), followed by FITC-conjugated antibody to rat IgG and Cy3-conjugated antibody to mouse IgG, and were analyzed by confocal laser scanning microscopy (Zeiss).

### Communoprecipitation of CXCR2 and CD74.

HEK293-CXCR2 cells transiently transfected with pcDNA3.1/V5-HisTOPO-TA-CD74 were lysed in nondenaturing CoIP buffer. Supernatants were incubated with the CXCR2 antibody RII115 or an isotype control, and were preblocked with protein G-sepharose overnight. Proteins were analyzed by western blots using an antibody to the His-tag (Santa Cruz). Similarly, CoIPs and immunoblots were performed with antibodies to the His-tag and CXCR2, respectively. L1.2-CXCR2 cells were subjected to immunoprecipitation with antibody to CXCR2 and immunoblotting with an antibody to mouse CD74.

### Ex Vivo Perfusion and Intravital Microscopy of Carotid Arteries.

Mif^{-/-}Ldlr^{-/-} mice and Mif^{+/+}Ldlr^{-/-} littermate controls, crossbred from Mif^{-/-} (Fingerle-Rowson, G., et al. (2003) Proc. Natl. Acad. Sci. USA 100, 9354-9359) and Ldlr^{-/-} mice (Charles River), and Apoe^{-/-} mice were fed an atherogenic diet (21% fat; Altromin) for 6 weeks. All single knockout strains had been back-crossed in the C57BL/6 background ten times. Mif^{+/+} and Mif^{-/-} mice were treated with TNF-α (intraperitoneally (i.p.), 4 h). Explanted arteries were transferred onto the stage of an epifluorescence microscope and perfused at 4 μl/min with calcein-AM-labeled MonoMac6 cells treated with antibodies to CD74 or CXCR2, isotype control IgG, or left untreated (Huo, Y., et al. (2001) J. Clin. Invest. 108, 1307-1314). Untreated monocytic cells were perfused after blockade with antibody to MIF for 30 min. For intravital microscopy, rhodamine-G (Molecular Probes) was administered intravenously (i.v.), and carotid arteries were exposed in anesthetized mice. Arrest (>30 s) of labeled leukocytes was analyzed by epifluorescence microscopy (Zeiss Axiotech, 20× water immersion). All studies were approved by local authorities (Bezirksregierung Köln), and complied with German animal protection law Az: 50.203.2-AC 36, 19/05.

### Mouse Model of Atherosclerotic Disease Progression.

Apoe^{-/-} mice fed an atherogenic diet for 12 weeks were injected (3 injections per week, each 50 μg) with antibodies to MIF (NIHIIID.9), CXCL12 (79014) or CXCL1 (124014, R&D) (n=6-10 mice) for an additional 4 weeks. Aortic roots were fixed by in situ perfusion and atherosclerosis was quantified by staining transversal sections with Oil-Red-O. Relative macrophage and T-cell contents were determined by staining with antibodies to MOMA-2 (MCA519, Serotec) or to CD3 (PC3/188A, Dako) and FITC-conjugated antibody. In Mif^{-/-}Ldl^{-/-} and Mif^{+/+}Ldlr^{-/-} mice fed a chow diet for 30 weeks, the abundance of luminal monocytes and lesional macrophages in aortic roots was determined as described (Verschuren, L., et al. (2005) Arterioscler. Thromb. Vasc. Biol. 161-167).

### Cremaster Microcirculation Model.

Human MIF (1 μg) was injected intra-scrotally and the cremaster muscle was exteriorized in mice treated with antibody to CXCR2 (100 μg i.p.). After 4 h, intravital microscopy (Zeiss Axioplan; 20×) was performed in postcapillary venules (Gregory, J. L., et al. (2004) Arthritis Rheum. 50, 3023-3034; Keane, M. P., et al. (2004) J. Immunol. 172, 2853-2860). Adhesion was measured as leukocytes stationary for more than 30 s, emigration as the number of extravascular leukocytes per field.

### Bone Marrow Transplantation.

Femurs and tibias were aseptically removed from donor Il8rb^{-/-} (Jackson Laboratories) or BALB/c mice. The cells, flushed from the marrow cavities, were administered i.v. into Mif^{+/+} or Mif^{-/-} mice 24 h after ablative whole-body irradiation (Zernecke, A., et al. (2005) Circ. Res. 96, 784-791).

### Model of Acute Peritonitis.

Mice repopulated with Il8rb^{+/+} or Il8rb^{-/-} bone marrow were injected i.p. with MIF (200 ng). After 4 h, peritoneal lavage was performed and Gr-1⁺CD115⁻F4/80⁻ neutrophils were quantified by flow cytometry using the relevant conjugated antibodies.

### Statistical Analysis.

Statistical analysis was performed using either a one-way analysis of variance (ANOVA) and Newman-Keuls post-hoc test or an unpaired Student's t-test with Welch's correction (GraphPad Prism).

### Example 2

### Surface-Bound MIF Induced Monocyte Arrest Through CXCR2

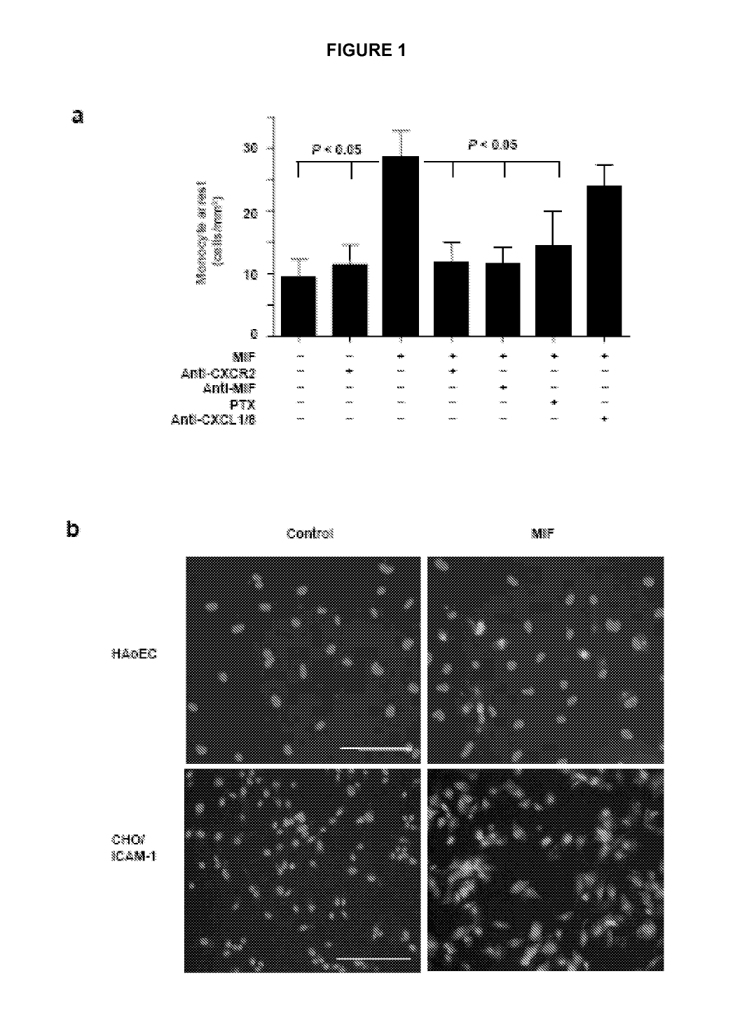

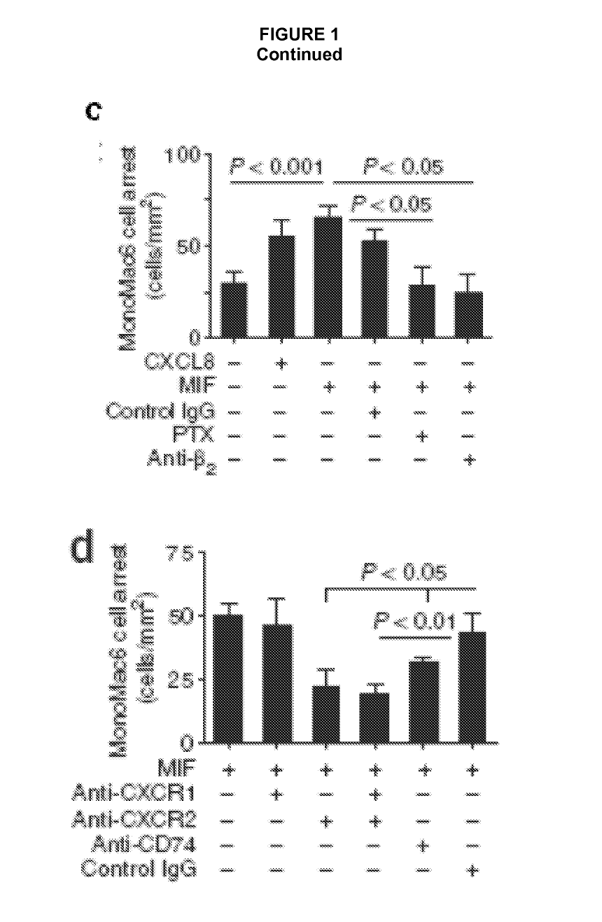

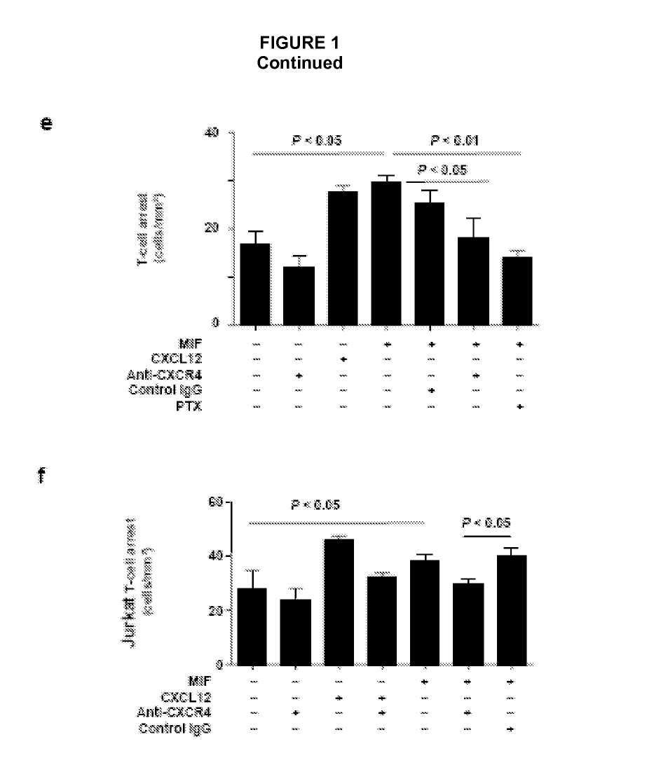

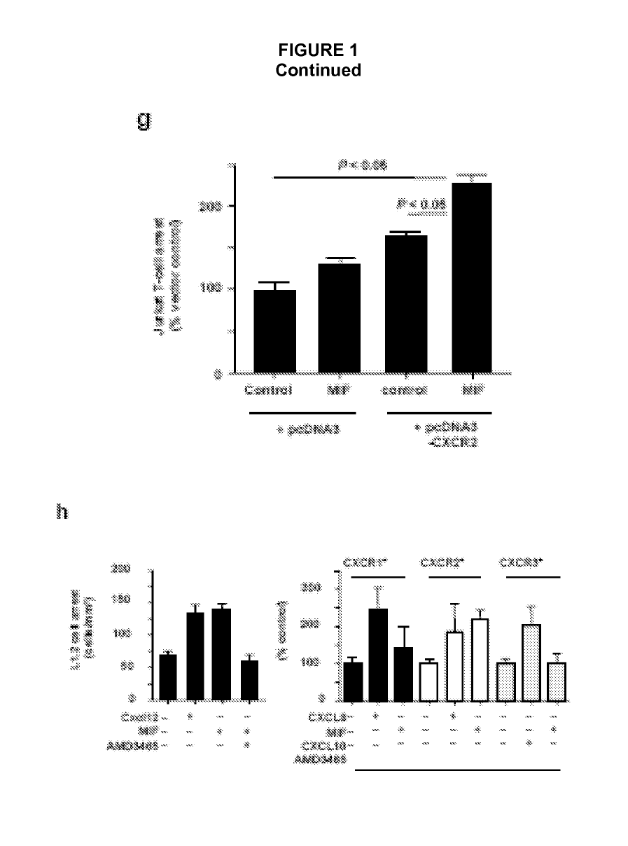

Monoclonal antibodies and pertussis toxin (PTX) were used to explore whether MIF-induced monocyte arrest depends on G_{αi}-coupled activities of CXCR2. Human aortic endothelial cells that had been pretreated with recombinant MIF for 2 h substantially increased the arrest of primary human monocytes under flow conditions, an effect blocked by an antibody to MIF (FIG. 1a). Notably, MIF-triggered, but not spontaneous, monocyte arrest was ablated by an antibody to CXCR2 or by PTX, implicating G_{αi}-coupled CXCR2. The ability of MIF to induce monocyte arrest through CXCR2 was confirmed using monocytic Mono-Mac6 cells and this activity was associated with an immobilization of MIF on aortic endothelial cells (FIG. 1b). This data indicated that MIF was presented on the endothelial cell surface and exerted a chemokine-like arrest function as a noncognate CXCR2 ligand. Blocking classical CXCR2 agonists (CXCL1/CXCL8) failed to interfere with these effects of MIF (FIG. 1a).

Chinese hamster ovary (CHO) transfectants that express the β₂ integrin ligand, ICAM-1 (intercellular adhesion molecule 1), were used to dissect the mechanisms by which MIF promotes integrin-dependent arrest. As quantified under flow conditions, the exposure of CHO transfectants to MIF for 2 h resulted in its surface presentation (FIG. 1b) and, like exposure of the transfectants to CXCL8, increased monocytic cell arrest (FIG. 1c). This effect was fully sensitive to PTX and an antibody to β₂ integrin (FIG. 1c), confirming a role of G_{αi} in β₂ integrin-mediated arrest induced by MIF. Primary monocytes and MonoMac6 cells express both CXCR1 and CXCR2 (Weber, K. S., et al. (1999) Eur. J. Immunol. 700-712). Whereas blocking CXCR1 had no effect, blocking CXCR2 substantially but not fully impaired MIF-triggered and CXCL8-triggered monocytic cell arrest. Addition of antibodies to both CXCR1 and CXCR2 completely inhibited the arrest functions of MIF or CXCL8 (FIG. 1d & FIG. 8). The use of antibodies to CD74 implicated this protein, along with CXCR2, in MIF-induced arrest (FIG. 1d). Spontaneous arrest was unaffected (FIG. 8). Thus, CXCR2 assisted by CD74 mediates MIF-induced arrest.

### MIF Induced T-Cell Arrest Through CXCR4

Either MIF or CXCL12 immobilized on aortic endothelial cells triggered the arrest of primary human effector T cells (FIG. 1e). MIF-induced, but not spontaneous, T-cell arrest was sensitive to PTX and was inhibited by an antibody to CXCR4 (FIG. 1e). Although less pronounced than in monocytes expressing CXCR2 (FIG. 1d), presentation of MIF (or CXCL12) on CHO transfectants expressing ICAM-1 elicited α_{L}β₂-dependent arrest of Jurkat T cells, an effect mediated by CXCR4 (FIG. 11).

Ectopic expression of CXCR2 in Jurkat T cells increased MIF-triggered arrest (FIG. 1g), corroborating the idea that CXCR2 imparts responsiveness to MIF in leukocytes. L1.2 pre-B lymphoma transfectants expressing CXCR1, CXCR2 or CXCR3, and controls using cells expressing endogenous CXCR4 only were used in the presence of the CXCR4 antagonist AMD3465. MIF triggered the arrest of CXCR2 transfectants and CXCR4-bearing controls on endothelial cells with a similar efficacy to that of the canonical ligands CXCL8 and CXCL12, whereas CXCR1 and CXCR3 transfectants were responsive to CXCL8 and CXCL10, respectively, but not to MIF (FIG. 1h). This data established that CXCR2 and CXCR4, but not CXCR1 or CXCR3, support MIF-induced arrest.

### Example 3

### MIF-Induced Leukocyte Chemotaxis Through CXCR2/4 Activation

Chemokines have been eponymously defined as inducers of chemotaxis (Baggiolini, M., et al. (1994) Adv. Immunol. 55, 97-179; Weber, C., et al. (2004) Arterioscler. Thromb. Vase. Biol. 24, 1997-2008). Paradoxically, MIF was initially thought to interfere with ‘random’ migration (Calandra, T., et al. (2003) Nat. Rev. Immunol. 3, 791-800). Although this may be attributable to active repulsion or desensitization of directed emigration, specific mechanisms evoked by MIF to regulate migration remain to be clarified. Our results showing that MIF induced G_{αI}-mediated functions of CXCR2 and CXCR4 prompted us to test if MIF directly elicits leukocyte chemotaxis through these receptors.

Using a transwell system, the promigratory effects of MIF and CXCL8 were compared on primary human peripheral blood mononuclear cell-derived monocytes. CCL2 was also used as a prototypic chemokine for monocytes. Similar to CXCL8 and CCL2, adding MIF to the lower chamber induced migration, which followed a bell-shaped dose-response curve typical for chemokines, with an optimum at 25-50 ng/ml, albeit with a lower peak migratory index (FIG. 2a). Heat treatment or a neutralizing antibody to MIF abolished MIF-induced transmigration. In contrast, isotype-matched immunoglobulin (IgG) had no effect (FIG. 2b). When added to the upper chamber, MIF dose-dependently desensitized migration toward MIF in the lower chamber (FIG. 2c) but did not elicit migration when present in the upper chamber only, suggesting that MIF evokes true chemotaxis rather than chemokinesis. Consistent with G_{αi}-dependent signaling through phosphoinositide-3-kinase, MIF-induced monocyte chemotaxis was sensitive to PTX and abrogated by Ly294002 (FIG. 2d). Both CXCR2 and CD74 specifically contributed to MIF-triggered monocyte chemotaxis (FIG. 2e). The role for CXCR2 was confirmed by showing MIF-mediated cross-desensitization of CXCL8-induced chemotaxis in CXCR2-transfected L1.2 cells. The chemotactic activity of MIF was verified in RAW264.7 macrophages (FIG. 8) and THP-1 monocytes. These data demonstrate that MIF triggers monocyte chemotaxis through CXCR2.

To substantiate functional MIF-CXCR4 interactions, the transmigration of primary CD3⁺ T lymphocytes devoid of CXCR1 and CXCR2 was evaluated. Similar to CXCL12, a known CXCR4 ligand and T-cell chemoattractant, MIF dose-dependently induced transmigration, a process that was chemotactic and transduced through CXCR4, as shown by antibody blockade and cross-desensitization of CXCL12 (FIG. 2f & FIG. 8). Thus, MIF elicits directed T-cell migration through CXCR4. In primary human neutrophils, a major cell type bearing CXCR2, MIF exerted CXCR2- but not CXCR1-mediated chemotactic activity, exhibiting a bell-shaped dose-response curve and cross-densensitizing CXCL8 (FIGS. 2g,h). The moderate chemotactic activity of neutrophils towards MIF is likely to be related to an absence of CD74 on neutrophils, as its ectopic expression in CD74⁻ promyelocytic HL-60 cells enhanced MIF-induced migration (FIG. 8). Although MIF, like other CXCR2 ligands, functions as an arrest chemokine, the present data revealed that MIF also has appreciable chemotactic properties on mononuclear cells and neutrophils.

### Example 4

### MIF Triggers Rapid Integrin Activation and Calcium Flux

Arrest functions of MIF may reflect direct MIF/CXCR signaling, but it cannot be entirely excluded that MIF induces other arrest chemokines during the time required for MIF immobilization. To consolidate evidence that MIF directly induces leukocyte arrest (FIG. 1), real-time PCR and ELISAs were performed and found that 2-h-long preincubation of human aortic (or venous) endothelial cells with MIF failed to upregulate typical arrest chemokines known to engage CXCR2 (FIG. 3a).

Short-term exposure to chemokines present in solution or immobilized in juxtaposition to integrin ligands (for example, vascular cell adhesion molecule (VCAM)-1) can rapidly upregulate integrin activity, which mediates leukocyte arrest (Laudanna, C., et al. (2006) Thromb. Haemost. 95, 5-11). This is accomplished by clustering (for example, α₄β₁) or conformational changes (for example, α_{L}β₂) immediately preceding ligand binding. Stimulation of monocytic cells with MIF (or CXCL8) for 1-5 min triggered α_{L}β₂-dependent arrest on CHO/CAM-1 cells (FIG. 3b). To obtain evidence for a direct stimulation of monocyte integrins, the reporter antibody 327C, which recognizes an extended high-affinity conformation of α_{L}β₂, was used (Shamri, R., et al. (2005) Nat. Immunol. 6, 497-506). These assays revealed that α_{L}β₂ activation in MonoMac6 cells (FIG. 3c) and human blood monocytes (FIG. 3d) occurred as early as 1 min after exposure to MIF and persisted over 30 min. To evaluate whether MIF's effects were restricted to β_{L}β₂, α₄β₁-dependent monocytic cell arrest on VCAM-1 was studied. Exposure to MIF for 1-5 min induced marked arrest, which was mediated by CXCR2, CD74 and α₄β₁ (FIG. 3e). Similarly to the effect of CXCL12, stimulation of Jurkat T cells with MIF for 1-5 min triggered CXCR4-dependent adhesion on VCAM-1 (FIG. 8).

As CXCR2 can mediate increases in cytosolic calcium elicited by CXCL8 (Jones, S. A., et al. (1997) J. Biol. Chem. 272, 16166-16169), the ability of MIF to stimulate calcium influx and desensitize CXCL8 signals was tested. Indeed, like CXCL8, MIF induced calcium influx in primary human neutrophils and desensitized calcium transients in response to either CXCL8 or MIF (FIG. 3f), confirming that MIF activates GPCR/G_{αi} signaling. The partial desensitization of CXCL8 signaling by MIF seen in neutrophils parallels findings with other CXCR2 ligands (Jones, S. A., et al. (1997) J. Biol. Chem. 272, 16166-16169) and reflects the presence of CXCR1. In L1.2 transfectants expressing CXCR2, MIF fully desensitized CXCL8-induced calcium influx, and in neutrophils, MIF desensitized transients induced by the selective CXCR2 ligand CXCL7 (and CXCL7 desensitized transients induced by MIF) (FIG. 3f). In CXCR2 transfectants, MIF dose-dependently induced calcium influx, and was slightly less potent and effective than CXCL8 or CXCL7 (FIG. 3g). In conclusion, MIF acted on CXCR2 and CXCR4 to elicit rapid integrin activation and calcium influx.

### Example 5

### MIF Interacts with CXCR2 and CXCR4

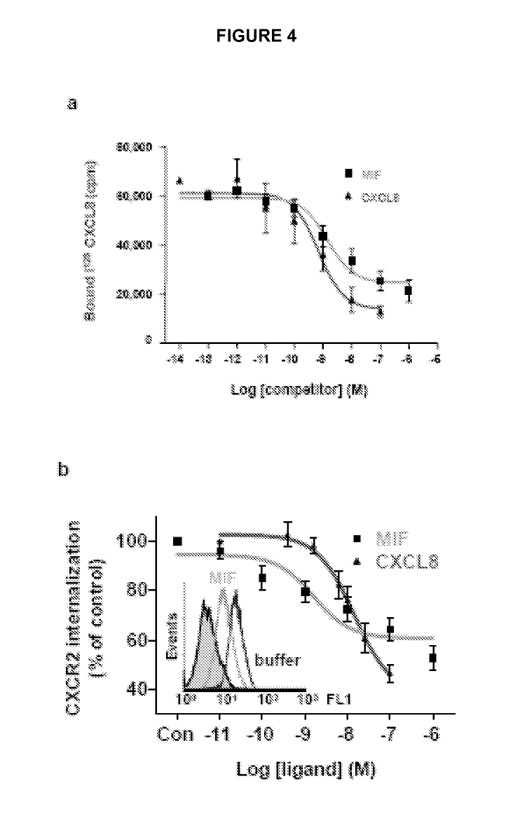

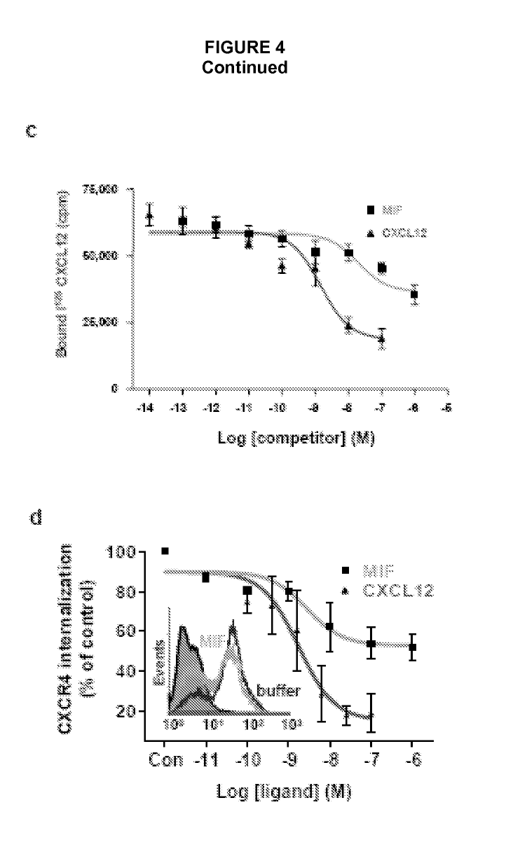

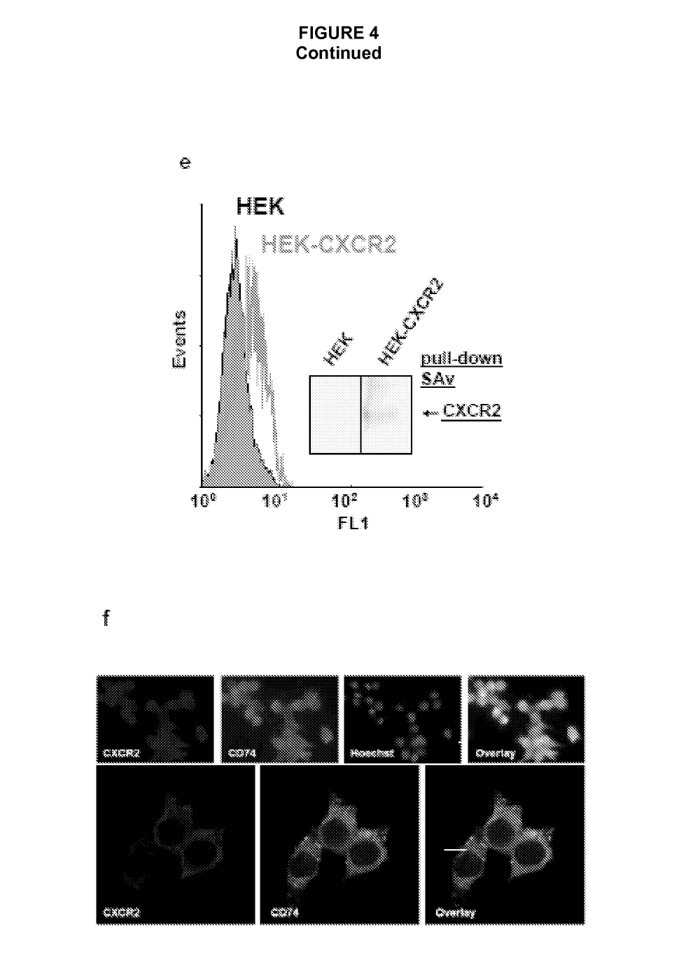

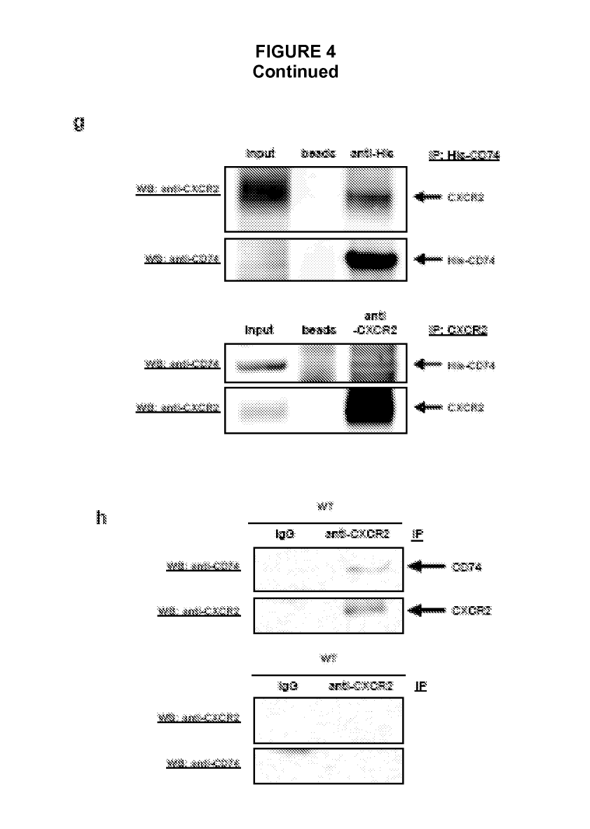

To assess the physical interactions of MIF with CXCR2 and CXCR4, we performed receptor-binding competition and internalization studies. In HEK293 cells ectopically expressing CXCR2, MIF strongly competed with ¹²⁴I-labeled CXCL8 for CXCR2 binding under equilibrium conditions. Binding of the CXCL8 tracer to CXCR2 was inhibited by MIF with an effector concentration for half-maximum response (EC₅₀) of 1.5 nM (FIG. 4a). The affinity of CXCR2 for MIF (K_{d}=1.4 nM) was close to that for CXCL8 (K_{d}=0.7 nM) and within the range of the MIF concentration that induced optimal chemotaxis (2-4 nM). To confirm binding to CXCR2, we used a receptor internalization assay that reports specific receptor-ligand interactions. FACS analysis of surface CXCR2 on stable HEK293 transfectants showed that MIF induced CXCR2 internalization with a dose response resembling that of CXCL8 (FIG. 4b). Comparable data was obtained in CXCR2-transfected RAW264.7 macrophages (inset in FIG. 4b).

To verify an interaction of MIF with CXCR4, receptor-binding studies were performed in Jurkat T cells, which endogenously express CXCR4. MIF competed with ¹²⁵I-labeled CXCL12 for CXCR4 binding (K_{d} for CXCL12=1.5 nM; EC₅₀=19.9 nM, K_{d} for MIF=19.8 nM) (FIG. 4c). The K_{d} was in accordance with MIF concentrations that induce T-cell chemotaxis. Consistently, MIF, like CXCL12, elicited CXCR4 internalization in a dose-dependent fashion (FIG. 4d). MIF-induced internalization of CXCR2 and CXCR4 was specific to these receptors, as MIF, unlike the cognate ligand CCL5, was unable to induce CCR5 internalization in L1.2 CCR5 transfectants.

To corroborate its interactions with CXCRs, MIF was labeled with biotin or fluorescein, which, in contrast to iodinated MIF, allows for direct receptor-binding assays. CXCR2 transfectants, but not vector controls, supported direct binding of labeled MIF, as evidenced by flow cytometry (FIG. 4e), pull down with streptavidin beads (inset in FIG. 4e) and fluorescence microscopy. In addition, the specific binding of fluorescein-MIF to CXCR4-bearing Jurkat cells was inhibited by the CXCR4 antagonist AMD3465.

### Complex Formation Between CXCR2 and CD74

Our data suggests the possibility that a functional MIF receptor complex involves both GPCRs and CD74. Thus, the colocalization of endogenous CD74 and CXCR2 was visualized using confocal fluorescence microscopy in RAW264.7 macrophages expressing human CXCR2. Using this technique, prominent colocalization was observed in a polarized pattern in ˜50% of cells (FIG. 4f).

In addition, coimmunoprecipitation assays revealed that CXCR2 physically interacts with CD74. CXCR2/CD74 complexes were detected in HEK293 cells stably overexpressing CXCR2 and transiently expressing His-tagged CD74. These complexes were observed by precipitation with an antibody to CXCR2 and by detecting coprecipitated CD74 by western blot against the His-tag. Coprecipitation was also seen when the order of the antibodies used was reversed (FIG. 4g). Complexes were also detected with CD74 in L1.2 transfectants stably expressing human CXCR2, as assessed by coimmunoprecipitation with an antibody to CXCR2. In contrast, no complexes were observed with L1.2 controls or the isotype control (FIG. 4h). The data are consistent with a model in which CD74 forms a signaling complex with CXCR2 to mediate MIF functions.

### Example 6

### CXCR2 Mediates IF-Induced Monocyte Arrest in Arteries

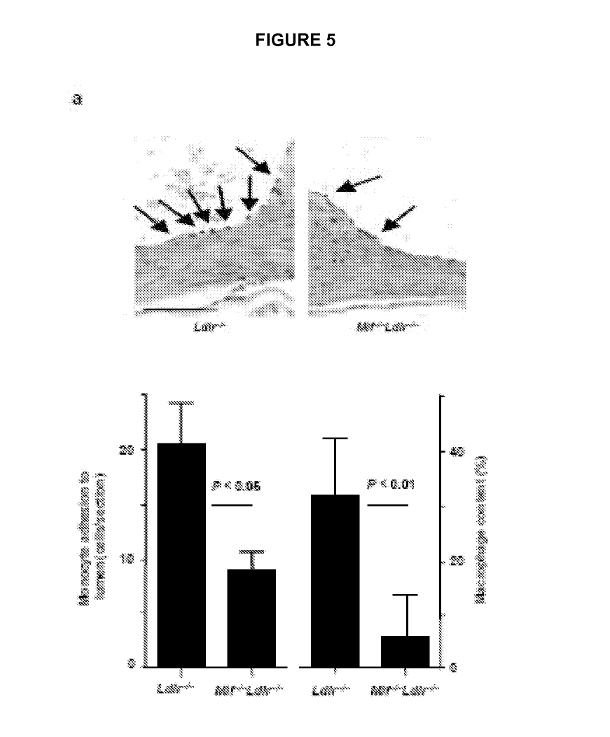

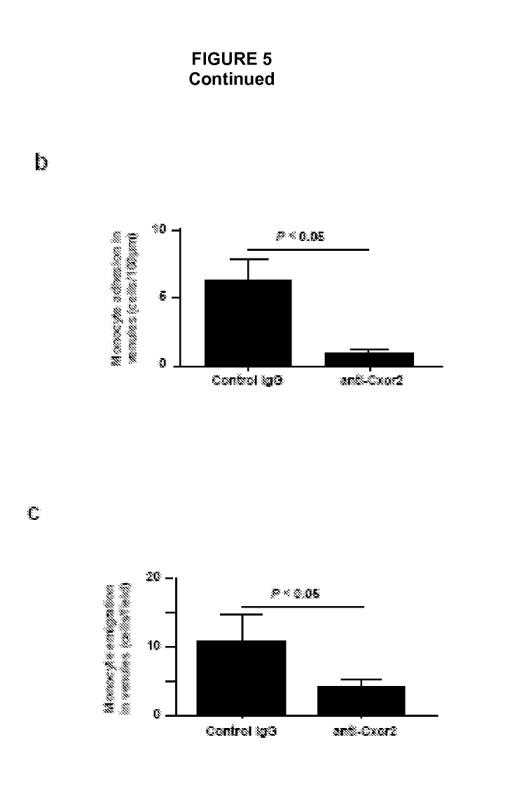

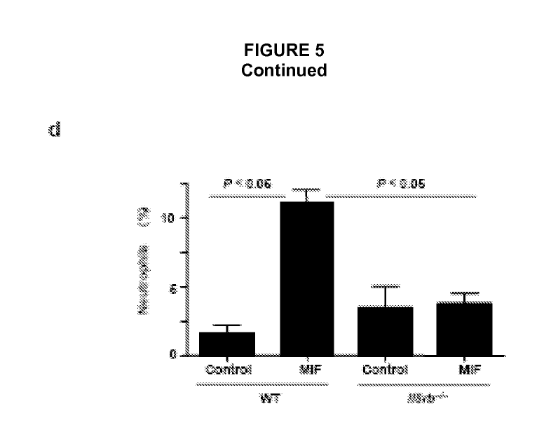

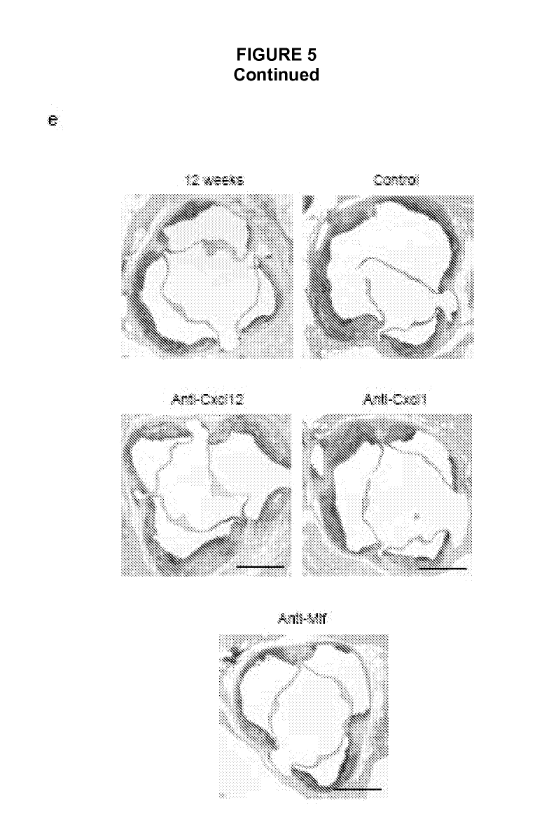

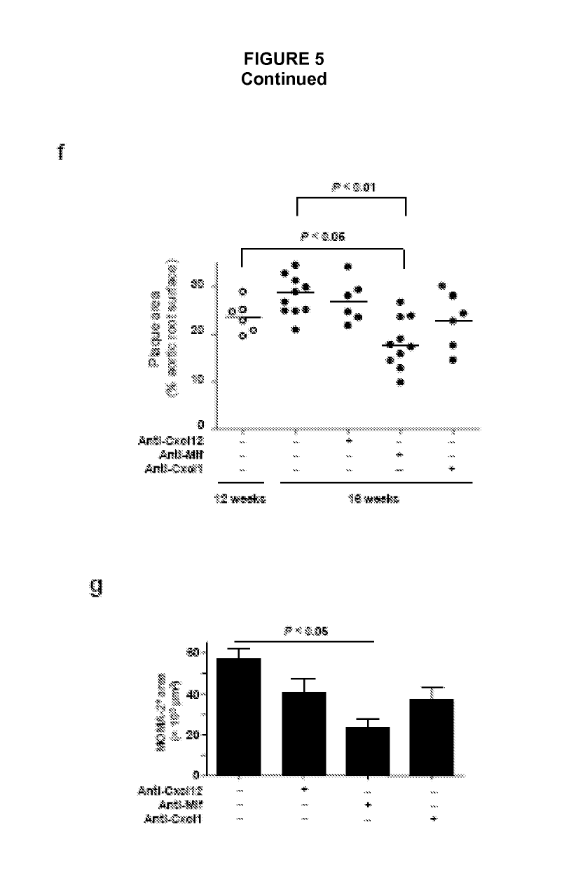

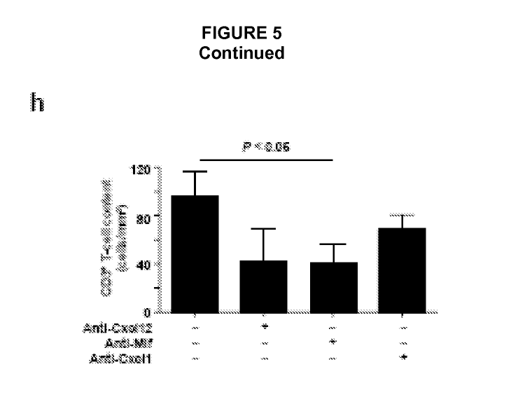

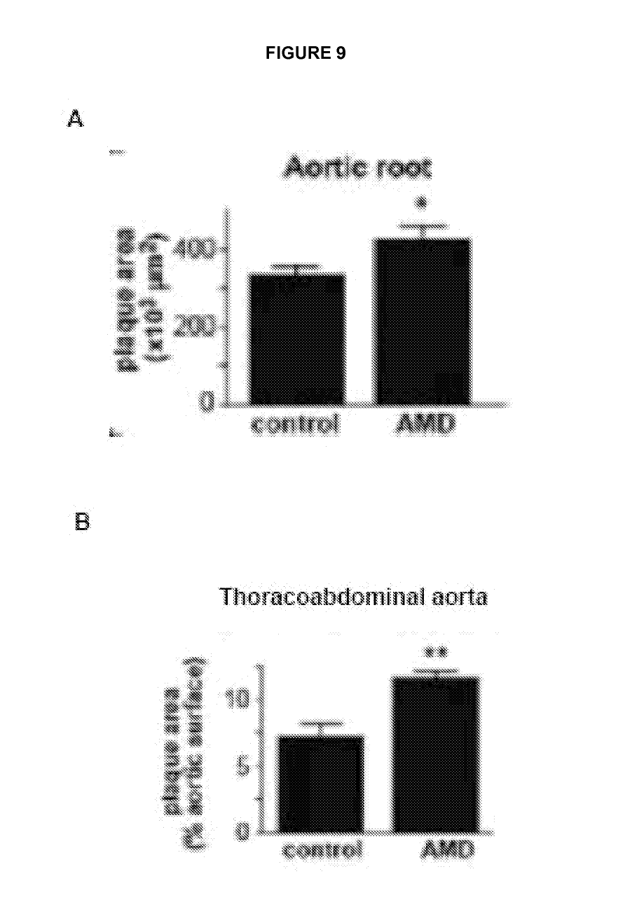

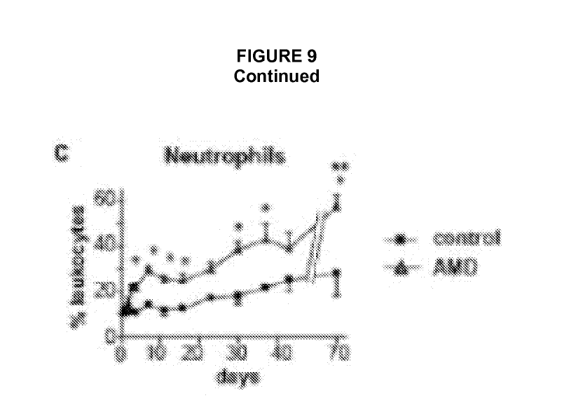

MIF promotes the formation of complex plaques with abundant cell proliferation, macrophage infiltration and lipid deposition (Weber, C., et al. (2004) Arterioscler. Thromb. Vasc. Biol. 24, 1997-2008; Morand, E. F., et al. (2006) Nat. Rev. Drug Discov. 5, 399-410). This has been related to the induction of endothelial MIF by oxLDL, triggering monocyte arrest (Schober, A., et al. (2004) Circulation 109, 380-385). The CXCR2 ligand CXCL1 can also elicit α₄β₁-dependent monocyte accumulation in ex vivo-perfused carotid arteries of mice with early atherosclerotic endothelium (Huo, Y., et al. (2001) J. Clin. Invest. 108, 1307-1314). This system was used to test whether MIF acts via CXCR2 to induce recruitment. Monocyte arrest in carotid arteries of Apoe^{-/-} mice fed a high-fat diet was inhibited by antibodies to CXCR2, CD74 or MIF (FIG. 5a & FIG. 9), indicating that MIF contributed to atherogenic recruitment via CXCR2 and CD74. Following the blockade of MIF, CXCR2 and CD74 for 24 h, a similar pattern was observed for monocyte arrest in arteries of wild-type mice treated with tumor necrosis factor (TNF)-α, mimicking acute vascular inflammation (FIG. 5b). In arteries of TNF-α-treated Mif^{-/-} mice, inhibitory effects on CD74 were attenuated and blocking MIF was ineffective, whereas there was residual CXCR2 inhibition, implying the involvement of other inducible ligands (FIG. 5c). Compared to the effect of MIF deficiency observed with TNF-α stimulation, monocyte accumulation was more clearly impaired by MIF deficiency in arteries of Mif^{-/-}Ldlr^{-/-} mice (compared to atherogenic Mif^{+/+}Ldlr^{-/-} mice; FIGS. 5d,e). In the absence of MIF, there was no apparent contribution of CXCR2. Moreover, blocking MIF had no effect (FIGS. 5d,e). The inhibitory effects of blocking CXCR2 were restored by loading exogenous MIF (FIG. 5f).

To provide further evidence for the idea that CXCR2 is required for MIF-mediated monocyte recruitment in vivo, intravital microscopy was performed on carotid arteries of chimeric wild-type Mif^{+/+} and Mif^{-/-} mice reconstituted with wild-type or Il8rb^{-/-} bone marrow (Il8rb encodes CXCR2; FIGS. 5g,h). After treatment with TNF-α for 4 h, the accumulation of rhodamine G-labeled leukocytes was attenuated in Mif^{-/-} mice reconstituted with wild-type bone marrow compared to that in wild-type mice reconstituted with wild-type bone marrow. The reduction in leukocyte accumulation due to deficiency in bone marrow CXCR2 was more marked in chimeric wild-type mice than in chimeric Mif^{-/-} mice (FIGS. 5g,h).

### Example 7

### MIF-Induced Inflammation In Vivo Relied on CXCR2

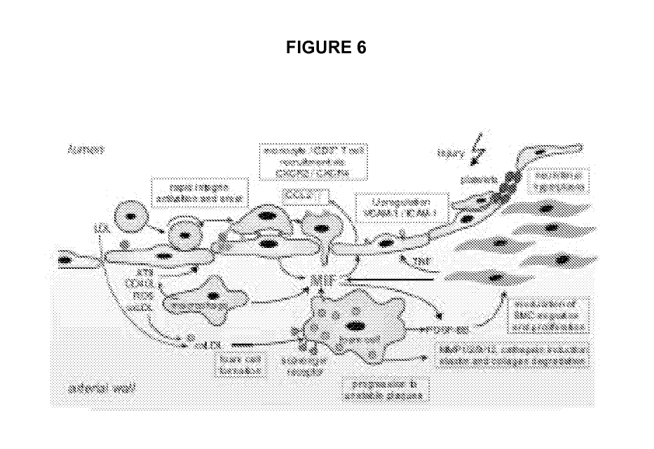

The importance of CXCR2 for MIF-mediated leukocyte recruitment under atherogenic or inflammatory conditions was corroborated in vivo. The adhesion of monocytes to the luminal surface of aortic roots was reduced in Mif^{-/-}Ldlr^{-/-} versus Mif^{+/+}Ldlr^{-/-} mice with primary atherosclerosis, and this was mirrored by a marked decrease in lesional macrophage content (FIG. 6a). Intravital microscopy of microcirculation in the cremaster muscle revealed that injecting MIF adjacent to the muscle caused a marked increase in (mostly CD68⁺) leukocyte adhesion and emigration in postcapillary venules, which was inhibited by an antibody to CXCR2 (FIGS. 6b,c).

Circulating Monocyte Counts were Unaffected.

Next a model of MIF-induced peritonitis was used in chimeric mice reconstituted with wild-type or Il8rb−/− bone marrow. Intraperitoneal injection of MIF elicited neutrophil recruitment after 4 h in mice with wild-type bone marrow, which was abrogated in mice with Il8rb^{-/-} bone marrow (FIG. 6d). Collectively, these results demonstrated that MIF triggers leukocyte recruitment under atherogenic and inflammatory conditions in vivo through CXCR2.

### Targeting MIF Resulted in Regression of Atherosclerosis

As described herein, MIF acted through both CXCR2 and CXCR4. Given the role of MIF and CXCR2 in the development of atherosclerotic lesions, targeting MIF, rather than CXCL1 or CXCL12, was investigated as a method to modify advanced lesions and their content of CXCR2⁺ monocytes and CXCR4⁺ T cells. Apoe^{-/-} mice, which had received a high-fat diet for 12 weeks and had developed severe atherosclerotic lesions, were treated with neutralizing antibodies to MIF, CXCL1 or CXCL12 for 4 weeks. Immunoblotting and adhesion assays were used to verify the specificity of the MIF antibody. These assays confirmed that the MIF antibody blocked MIF-induced, but not CXCL1- or CXCL8-induced, arrest (FIG. 10).

Blockade of MIF, but not CXCL1 or CXCL12, resulted in a reduced plaque area in the aortic root at 16 weeks and a significant (P<0.05) plaque regression compared to baseline at 12 weeks (FIGS. 6e,f). In addition, blockade of M IF, but not CXCL1 or CXCL12, was associated with less of an inflammatory plaque phenotype at 16 weeks, as evidenced by a lower content of both macrophages and CD3* T cells (FIGS. 6g,h). Therefore, by targeting MIF and inhibiting the activation of CXCR2 and CXCR4, therapeutic regression and stabilization of advanced atherosclerotic lesions was achieved. In some embodiments, the present invention comprises a method of reducing plaque area in an individual in need thereof, comprising administering to said individual one or more agents that inhibit (i) MIF binding to CXCR2 and/or CXCR4 and/or (ii) MIF-activation of CXCR2 and/or CXCR4; or (iii) any combination of (i) and (ii).

### Example 8

### Interference with CXCR4 Aggravates Atherosclerosis

To explore the role of CXCR4 in atherosclerosis, Apoe−/− mice fed an atherogenic diet are continuously treated with the CXCR4 antagonist AMD3465 or vehicle (controls) via osmotic minipumps, and atherosclerotic plaque formation is analyzed after 12 weeks. Compared with controls, AMD3465 treatment significantly exacerbates lesion formation in oil red O-stained aortic root sections (FIG. 9a) and in thoracoabdominal aortas prepared en face (FIG. 9b). In addition continuous treatment of Apoe−/− mice with AMD3465 induces a pronounced peripheral blood leukocytosis within 2 days, which is sustained throughout the study period, and an expansion in the relative number of circulating neutrophils, which further increases during disease progression (FIG. 9c).

### Example 9

### Blocking Th-17 Development in a Mouse Model of Multiple Sclerosis

Eight- to twelve-week-old C57BL/6 mice (obtained from The Jackson Laboratory, Bar Harbor, Main, USA) are pretreated on day −1 and weekly thereafter with intraperitoneal injections of 5 mg/kg of either a control antibody (group 1), an antagonistic anti-mouse MIF antibody (group 2), an antibody to CXCR2 that blocks MIF binding and/or activation of CXCR2 (group 3), an antibody to CXCR4 that blocks MIF binding and/or activation of CXCR4 (group 4) or an antibody to CXCR4 that blocks MIF binding and/or activation of CXCR4 and an antibody to CXCR2 that blocks MIF binding and/or activation of CXCR2 (group 5). Mice (n=30 per group) are immunized the following day (day 0) by two subcutaneous injections on the back totaling 200 μl of an emulsification of MOG35-55 peptide (MEVGWYRSPFSRVVHLYRNGK; Bachem AG, Bubendorf, Switzerland) in CFA. The final concentrations of peptide and M. tuberculosis are 150 μg/mouse and 1 mg/mouse, respectively. PTX (400 ng; LIST Biological Laboratories Inc., Campbell, California, USA) is injected intraperitoneally on days 0 and 2. The disease is monitored daily by measuring paralysis on a 0-6 scale as described above. Average maximal disease scores are compared between groups using a one-way ANOVA.

Paralysis measurements are compared between group 2 mice and group 1 to determine the efficacy of an antagonistic anti-MIF antibody, for treating or preventing EAE. Group 5 mice are compared to group 1 mice to determine the efficacy of an agent that blocks MIF binding and/or activation of CXCR2 and CXCR4, for treating or preventing EAE. Group 5 mice are compared to groups 3 & 4 to determine the effect of blocking MIF binding and/or activation of both CXCR2 and CXCR4 to the effect of blocking CXCR2 or CXCR4 individually.

Mixed T cells are prepared from draining lymph nodes and spleen on day 7-11 after immunization. Viable cells (3.75×10⁶/ml) are cultured in complete medium with (re-stimulated) or without MOG peptide (amino acids 35-55) at various concentrations. Supernatants from activated cells are collected 72 h later and TNF, IFN-γ, IL-23 & IL-17 are measured by ELISA (BD Pharmingen). High IL-17 and IL-23 levels indicate the development of a Th-17 cells and a Th-17 mediated disease phenotype. Inhibition of these cytokines by treatment of mice or cell cultures with MIF blocking antibodies (group 2), or by blocking MIF binding and/or activation of both CXCR2 and CXCR4 (group 5) illustrates a key regulatory role of MIF in the development of Th-17 cells and in the progression of a Th-17 mediated inflammatory disease (i.e. multiple sclerosis).

For intracellular cytokine staining, spleen and lymph node cells from immunized mice are stimulated for 24 h with peptide antigen, and GolgiPlug (BD Pharmingen) is added in the last 5 h or GolgiPlug plus 500 ng/ml of ionomycin and 50 ng/ml of phorbol 12-myristate 13-acetate (PMA; Sigma-Aldrich) are added for 5 h. For cell staining, cells are permeabilized with the Cytofix/Cytoperm Plus Kit (BD Pharmingen) according to the manufacturer's protocol. Gated CD4-positive T-cells are analyzed for the presence of intracellular IL-17, IL-23 or cell surface IL23 receptor (IL23R) by flow cytometry. The presence of CD4+, IL-17+ double positive T-cells indicates development of a Th-17 phenotype that is driving disease progression. Further the up-regulation of IL-23Rs on CD4+, IL-17 double positive cells provides supportive evidence of a Th-17 phenotype. The presence of high intracellular IL-23 in CD4+, IL-17 double positive cells or in any leukocyte provides additional supportive evidence for IL-23 driving Th-17 cell expansion and/or maintenance. Inhibition of Th-17 cell development, as determined by lower levels of IL-17, IL-23R or IL-23, as described in the above experiment, by treating mice with MIF blocking agents (group 2 mice) or agents that block MIF binding/or activation of CXCR2 and CXCR4 (group 5 mice) demonstrates a dominant role for MIF in driving the progression of Th-17 mediated autoimmune disease. The inhibition of Th-17 cell development and the inhibition of the progression of EAE in mice by blocking MIF demonstrates the valuable utility of agents that inhibit (i) MIF binding to CXCR2 and/or CXCR4 and/or (ii) MIF-activation of CXCR2 and/or CXCR4; or (iii) any combination of (i) and (ii) for the treatment and/or prevention of Th-17 mediated autoimmune diseases such as multiple sclerosis.

### Example 10

### Human Clinical Trial for Treatment of Homozygous Familial Hypercholesterolemia

Study Objective(s): The primary objective of this study is to assess efficacy of anti-MIF antibody 1 (AB1) in individuals with homozygous familial hypercholesterolemia (HoFH). AB1 specifically binds to the MIF peptide sequence DQLMAFGGSSEPCALCSL.

### Methods

Study Design: This is a multi-center, open-label, single-group study of AB1 in male and female individuals ≧18 years of age with HoFH. After initial screening, eligible individuals enter a 4-week screening period, consisting of 2 visits (Weeks −4 and −1), during which all lipid-lowering drugs are discontinued (except for bile acid sequestrants and cholesterol absorption inhibitors) and therapeutic lifestyle change counseling (TLC) according to National Cholesterol Education Program (NCEP) Adult Treatment Panel (ATP-III) clinical guidelines or equivalent are initiated. Individuals already on apheresis continue their treatment regimen maintaining consistent conditions and intervals during the study. At Visit 3 (Week 0), baseline efficacy/safety values are determined and individuals begin treatment with the initial dose of AB1. Treatment frequency is once per week, for 12 weeks. Study visits are timed with individuals' apheresis treatments to occur immediately before the visit procedures, where applicable. When the intervals between aphereses are misaligned with a study drug treatment period, the individuals are kept in the same drug treatment period until the next scheduled apheresis, and until the intervals are brought back to the original length of time. Efficacy measures are done at least 2 weeks after the previous apheresis and just before the apheresis procedure scheduled for the day of study visit.

Number of Participants: Between 30 and 50 individuals.

Diagnosis and Main Criteria for Inclusion: Men and women 18 years of age or older with definite evidence of the familial hypercholesterolemia (FH) homozygote per World Health Organization guidelines, and with serum fasting triglyceride (TG) ≦400 mg/dL (4.52 mmol/L) for individuals aged >20 years and 200 mg/dL (2.26 mmol/L) for individuals aged 18-20 years, are screened for study participation.

Study Treatment: The initial administration of AB1 is infused into subject at a rate of 50 mg/hr. In the absence of infusion toxicity, increase infusion rate by 50 mg/hr increments every 30 minutes, to a maximum of 400 mg/hr. Each week thereafter, AB1 is infused at a rate of 100 mg/hr. In the absence of infusion toxicity, increase rate by 100 mg/hr increments at 30-minute intervals, to a maximum of 400 mg/hr.

Efficacy Evaluations: The primary endpoints are the mean percent changes in HDL-C and LDL-C from baseline to week 3, week 6, and week 12. A lipid profile which includes HDL-C and LDL-C is obtained at each study visit.

### Example 11

### Animal Model for Treatment of Aortic Aneurysms (AAA)

Animal models are prepared as follows. An adult, male rat at is subjected to infusion of elastase for 2 hours. Histological analysis is performed 12-24 hours after infusion to confirm presence of fragmented and disorganized elastin. Ultrasound is performed daily to identify and monitor areas of aortic enlargement.

2 weeks after administration of elastase, the rat is administered AB1 (binds to the MIF peptide sequence DQLMAFGGSSEPCALCSL). The initial administration of AB1 is infused into subject at a rate of 0.5 mg/hr. In the absence of infusion toxicity, increase infusion rate by 0.5 mg/hr increments every 30 minutes, to a maximum of 2.0 mg/hr. Each week thereafter, AB1 is infused at a rate of 1.0 mg/hr. In the absence of infusion toxicity, increase rate by 1.0 mg/hr increments at 30-minute intervals, to a maximum of 4.0 mg/hr.

Efficacy Evaluations: The primary endpoints are the mean percent changes in AAA size (i.e., aortic diameter) from baseline to weeks 3, 6, and 12.

### Example 12

### Human Clinical Trial for Treatment of Abdominal Aortic Aneurysms (AAA)

Study Objective(s): The primary objective of this study is to assess efficacy of anti-MIF antibody 1 (AB1) in individuals with early AAA. AB1 specifically binds to the MIF peptide sequence DQLMAFGGSSEPCALCSL.

### Methods

Study Design: This is a multi-center, open-label, single-group study of AB1 in male and female individuals ≧18 years of age with early AAA. Presence of early AAA is confirmed with serial cross-sectional imaging. At Week 0, baseline efficacy/safety values are determined and individuals begin treatment with the initial dose of AB1. Subjects are administered AB1 once a week for 12 weeks.

Number of Participants: Between 30 and 50 individuals.

Study Treatment: The initial administration of AB1 is infused into subject at a rate of 50 mg/hr. In the absence of infusion toxicity, increase infusion rate by 50 mg/hr increments every 30 minutes, to a maximum of 400 mg/hr. Each week thereafter, AB1 is infused at a rate of 100 mg/hr. In the absence of infusion toxicity, increase rate by 100 mg/hr increments at 30-minute intervals, to a maximum of 400 mg/hr.

Efficacy Evaluations: The primary endpoints are the mean percent changes in AAA size (i.e., aortic diameter) from baseline to weeks 3, 6, and 12.

### Example 13

### Raising Polyclonal Anti-MIF Antibodies

Antibodies are generated against the following peptide sequence: PRASVPDGFLSELTQQLAQATGKPPQYIAVHVVPDQLMAFGGSSEPCALCSL. New Zealand White rabbits are the host animal used to generate the antibodies.

A peptide BSA conjugate is generated via GMBS conjugation. The conjugate is then formulated as a solution using Freund's complete adjuvant.

On Day 0, the rabbits are bled (25 mL). Then the rabbits are immunized with 0.2 mg of the antigenic composition. On day 21, the rabbits are administered an additional dose of is 0.1 mg. On day 32, the rabbits are bled (25 mls). The interaction of antibodies raised against the specific antigens of a MIF monomer or MIF trimer is confirmed by comparing interaction of serum from the rabbits obtained on day 0 with interaction of serum from the rabbits obtained on day 32 by Western blot.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a MIF-mediated disorder comprising administering to an individual in need thereof a therapeutically-effective amount of an antibody that inhibits (i) MIF binding to CXCR2 and/or CXCR4 (ii) MIF-activation of CXCR2 and/or CXCR4; (iii) the ability of MIF to form a homomultimer; (iv) MIF binding to CD74; or a combination thereof.

2. The method of claim 1, wherein the antibody specifically binds to all or a portion of or competes with an N-Loop motif of MIF.

3. The method of claim 1, wherein the antibody specifically binds to all or a portion of the pseudo-ELR and N-Loop motifs of MIF.

4. The method of claim 1, wherein the antibody is selected from an anti-CXCR2 antibody; an anti-CXCR4 antibody; an anti-MIF antibody; an antibody that specifically binds to all or a portion of the N-loop motif of MIF; an antibody that specifically binds to all or a portion of the pseudo-ELR and N-Loop motifs; an antibody that inhibits the binding of MIF and CXCR2; an antibody that inhibits the binding of MIF and CXCR4; and antibody that inhibits the binding of MIF and JAB-1; an antibody that inhibits the binding of MIF and CD74; an antibody that specifically binds to all or a portion of a peptide sequence as follows: DQLMAFGGSSEPCALCSL and the corresponding feature/domain of at least one of a MIF monomer or MIF trimer; an antibody that specifically binds to all or a portion of a peptide sequence as follows: PRASVPDGFLSELTQQLAQATGKPPQYIAVHVVPDQLMAFGGSSEPCALCSL and the corresponding feature/domain of at least one of a MIF monomer or MIF trimer; an antibody that specifically binds to all or a portion of a peptide sequence as follows: FGGSSEPCALCSLHSI and the corresponding feature/domain of at least one of a MIF monomer or MIF trimer; or combinations thereof.

5. The method of claim 1, wherein the antibody is selected from anti-CXCR4 antibodies 701, 708, 716, 717, 718, 12G5 and 4G10; anti-MIF antibodies IID.9, IIID.9, XIF7, I31, IV2.2, XI17, XIV14.3, XII15.6 and XIV15.4; or combinations thereof.

6. The method of claim 1, wherein the conversion of a macrophage into a foam cell is inhibited following administration of an antibody disclosed herein.

7. The method of claim 1, wherein apoptosis of a cardiac myocyte is inhibited following administration of an antibody disclosed herein.

8. The method of claim 1, wherein apoptosis of an infiltrating macrophage is inhibited following administration of an antibody disclosed herein.

9. The method of claim 1, wherein the formation of an abdominal aortic aneurysm is inhibited following administration of an antibody disclosed herein.

10. The method of claim 1, wherein the diameter of an abdominal aortic aneurysm is decreased following administration of an antibody disclosed herein.

11. The method of claim 1, wherein a structural protein in an aneurysm is regenerated following administration of an antibody disclosed herein.

12. The method of claim 1, further comprising co-administering a second active agent.

13. The method of claim 1, further comprising co-administering niacin, a fibrate, a statin, a Apo-A1 mimetic peptide (e.g., DF-4, Novartis), an apoA-I transcriptional up-regulator, an ACAT inhibitor, a CETP modulator, Glycoprotein (GP) IIb/IIIa receptor antagonists, P2Y12 receptor antagonists, Lp-PLA2-inhibitors, an anti-TNF agent, an IL-1 receptor antagonist, an IL-2 receptor antagonist, a cytotoxic agent, an immunomodulatory agent, an antibiotic, a T-cell co-stimulatory blocker, a disorder-modifying anti-rheumatic agent, a B cell depleting agent, an immunosuppressive agent, an anti-lymphocyte antibody, an alkylating agent, an anti-metabolite, a plant alkaloid, a terpenoids, a topoisomerase inhibitor, an anti-tumor antibiotic, a monoclonal antibody, a hormonal therapy, or combinations thereof.

14. The method of claim 1; wherein the disorder is Atherosclerosis; Abdominal aortic aneurysm; Acute disseminated encephalomyelitis; Moyamoya disease; Takayasu disease; Acute coronary syndrome; Cardiac-allograft vasculopathy; Pulmonary inflammation; Acute respiratory distress syndrome; Pulmonary fibrosis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome; Multiple sclerosis; Myasthenia gravis; Myocarditis; Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder; Endotoxin shock; Septic shock; Rheumatoid spondylitis; Ankylosing spondylitis; Gouty arthritis; Polymyalgia rheumatica; Alzheimer's disorder; Parkinson's disorder; Epilepsy; AIDS dementia; Asthma; Adult respiratory distress syndrome; Bronchitis; Cystic fibrosis; Acute leukocyte-mediated lung injury; Distal proctitis; Wegener's granulomatosis; Fibromyalgia; Bronchitis; Uveitis; Conjunctivitis; Psoriasis; Eczema; Dermatitis; Smooth muscle proliferation disorders; Meningitis; Shingles; Encephalitis; Nephritis; Tuberculosis; Retinitis; Atopic dermatitis; Pancreatitis; Periodontal gingivitis; Coagulative Necrosis; Liquefactive Necrosis; Fibrinoid Necrosis; Neointimal hyperplasia; Myocardial infarction; Stroke; Organ transplant rejection; or combinations thereof.

15. A pharmaceutical composition for the treatment of a MIF-mediated disorder, comprising an antibody that inhibits (i) MIF binding to CXCR2 and CXCR4 and/or (ii) MIF-activation of CXCR2 and CXCR4; (iii) the ability of MIF to form a homomultimer; or a combination thereof.

16. The composition of claim 15, wherein the antibody specifically binds to all or a portion of a N-Loop motif of MIF.

17. The composition of claim 15, wherein the antibody specifically binds to all or a portion of the pseudo-ELR and N-Loop motifs of MIF.

18. The composition of claim 15, wherein the antibody is selected from an anti-CXCR2 antibody; an anti-CXCR4 antibody; an anti-MIF antibody; an antibody that specifically binds to all or a portion of the N-loop motif of MIF; an antibody that specifically binds to all or a portion of the pseudo-ELR and N-Loop motifs; an antibody that inhibits the binding of MIF and CXCR2; an antibody that inhibits the binding of MIF and CXCR4; and antibody that inhibits the binding of MIF and JAB-1; an antibody that inhibits the binding of MIF and CD74; an antibody that specifically binds to all or a portion of a peptide sequence as follows: DQLMAFGGSSEPCALCSL and the corresponding feature/domain of at least one of a MIF monomer or MIF trimer; an antibody that specifically binds to all or a portion of a peptide sequence as follows: PRASVPDGFLSELTQQLAQATGKPPQYIAVHVVPDQLMAFGGSSEPCALCSL and the corresponding feature/domain of at least one of a MIF monomer or MIF trimer; an antibody that specifically binds to all or a portion of a peptide sequence as follows: FGGSSEPCALCSLHSI and the corresponding feature/domain of at least one of a MIF monomer or MIF trimer; or combinations thereof.

19. The composition of claim 15, wherein the antibody is selected from anti-CXCR4 antibodies 701, 708, 716, 717, 718, 12G5 and 4G10; anti-MIF antibodies IID.9, IIID.9, XIF7, I31, IV2.2, XI17, XIV14.3, XII15.6 and XIV15.4; or combinations thereof.

20. The composition of claim 15, further comprising a second active agent.

21. The composition of claim 15, further comprising niacin, a fibrate, a statin, a Apo-A1 mimetic peptide (e.g., DF-4, Novartis), an apoA-1 transcriptional up-regulator, an ACAT inhibitor, a CETP modulator, Glycoprotein (GP) IIb/IIIa receptor antagonists, P2Y12 receptor antagonists, Lp-PLA2-inhibitors, an anti-TNF agent, an IL-1 receptor antagonist, an IL-2 receptor antagonist, a cytotoxic agent, an immunomodulatory agent, an antibiotic, a T-cell co-stimulatory blocker, a disorder-modifying anti-rheumatic agent, a B cell depleting agent, an immunosuppressive agent, an anti-lymphocyte antibody, an alkylating agent, an anti-metabolite, a plant alkaloid, a terpenoids, a topoisomerase inhibitor, an anti-tumor antibiotic, a monoclonal antibody, a hormonal therapy, or combinations thereof.


SEQUENCE LISTING (7 sequences)

SEQ ID NO: 1 - protein, 116 aa, Homo sapiens
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFAL

SEQ ID NO: 2 - protein, 18 aa, Homo sapiens
DQLMAFGGSSEPCALCSL

SEQ ID NO: 3 - protein, 52 aa, Homo sapiens
PRASVPDGFLSELTQQLAQATGKPPQYIAVHVVPDQLMAFGGSSEPCALCSL

SEQ ID NO: 4 - protein, 16 aa, Homo sapiens
FGGSSEPCALCSLHSI

SEQ ID NO: 5 - protein, 36 aa, Homo sapiens
PRASVPDGFLSELTQQLAQATGKPPQYIAVHVVPDQ

SEQ ID NO: 6 - protein, 18 aa, Artificial Sequence
  Description of Artificial Sequence Synthetic peptide
DWFKAFYDKVAEKFKEAF

SEQ ID NO: 7 - protein, 21 aa, Artificial Sequence
  Description of Artificial Sequence Synthetic peptide
MEVGWYRSPFSRVVHLYRNGK